(19) United States
(12) Patent Application Publication
(10) Pub. No.: US 2006/0094038 A1
(43) Pub. Date: May 4, 2006

(54) Cardiac pressure overload associated genes

(75) Inventors: Roger A. Wagner, Stanford, CA (US); Raymond Tabibiazar, Stanford, CA (US); Thomas Quertermous, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Board of Trustees of the Leland Stanford Junior University

(21) Appl. No.: 11/231,700

(22) Filed: Sep. 20, 2005

Related U.S. Application Data
(60) Provisional application No. 60/611,674, filed on Sep. 20, 2004.

(51) Int. Cl. C12Q 1/68; G01N 33/53; C12M 1/34

(52) U.S. Cl. 435/6; 435/7.1; 435/287.2

(57) ABSTRACT

The present invention identifies genes whose gene products are differentially expressed pressure overload of the heart. The invention provides methods for diagnosing or assessing an individual's susceptibility to heart failure from many etiologies, as well as the presence and severity of hypertrophy, chamber enlargement, or systolic heat failure. Also provided are therapeutic methods for treating a heart patient or methods for prophylactically treating an individual susceptible to heart failure. Additionally, the invention describes screening methods for identifying agents that can be administered to treat individuals that have suffered a heart attack or are at risk of heart failure.

### INTRODUCTION

Heart failure is the leading cause of morbidity in western cultures. Congestive heart failure (CHF) develops when plasma volume increases and fluid accumulates in the lungs, abdominal organs (especially the liver), and peripheral tissues. In many forms of heart disease, the clinical manifestations of HF may reflect impairment of the left or right ventricle. Left ventricular (LV) failure characteristically develops in coronary artery disease, hypertension, cardiac valvular disease, many forms of cardiomyopathy, and with congenital defects. Right ventricular (RV) failure is most commonly caused by prior LV failure, which increases pulmonary venous pressure and leads to pulmonary arterial hypertension and tricuspid regurgitation. Heart failure is manifest by systolic or diastolic dysfunction, or both. Combined systolic and diastolic abnormalities are common.

In systolic dysfunction, primarily a problem of ventricular contractile dysfunction, the heart fails to provide tissues with adequate circulatory output. A wide variety of defects in energy utilization, energy supply, electrophysiologic functions, and contractile element interaction occur, which appear to reflect abnormalities in intracellular Ca⁺⁺ modulation and adenosine triphosphate (ATP) production. Systolic dysfunction has numerous causes; the most common are coronary artery disease, hypertension, valvular disease, and dilated cardiomyopathy. Additionally, there are many known and probably many unidentified causes for dilated myocardiopathy, e.g. virus infection, toxic substances such as alcohol, a variety of organic solvents, certain chemotherapeutic drugs (e.g., doxorubicin), β-blockers, Ca blockers, and antiarrhythmic drugs.

Diastolic dysfunction accounts for 20 to 40% of cases of heart failure. It is generally associated with prolonged ventricular relaxation time, as measured during isovolumic relaxation. Resistance to filling directly relates to ventricular diastolic pressure; this resistance increases with age, probably reflecting myocyte loss and increased interstitial collagen deposition. Diastolic dysfunction is presumed to be dominant in hypertrophic cardiomyopathy, circumstances with marked ventricular hypertrophy, e.g. hypertension, advanced aortic stenosis, and amyloid infiltration of the myocardium. Without intervention, hypertrophic cardiomyopathy and diastolic dysfunction often progress to systolic dysfunction and overt, symptomatic heart failure in the natural course of the disease.

The mammalian heart responds to pressure overload by undergoing left ventricular hypertrophy (LVH) and left atrial enlargement (LAE). These adaptive responses to increases in hemodynamic overload involve many alterations in myocardial structure and function. Although these responses are necessary in the short term to maintain cardiac output in the face of increased afterload, LVH and LAE are associated with increased risk for sudden death and progression to heart failure, the leading cause of morbidity in western cultures. A detailed understanding of the molecular events accompanying these changes is an important step toward the ability to interrupt or reverse their progression.

While the LV takes the brunt of the pressure insult, during pressure overload the left atrium faces physiological challenges due to mitral regurgitation and increased wall stress, which result in enlargement and remodeling. Many of the most important clinical complications of hypertrophic cardiomyopathy, valvulvar heart disease, and congestive heart failure are due to atrial enlargement, and include atrial fibrillation and other electrophysiological disturbances, as well as hemodynamic compromise caused by decreased ventricular filling. In humans, the hemodynamic and electrophysiological sequelae of left atrial enlargement are nearly as important as those stemming from LVH.

In view of the importance of cardiomyopathy for human mortality and morbidity, the identification of genes involved in the disease, and development of methods of treatment is of great interest.

### SUMMARY OF THE INVENTION

The present invention provides methods and compositions for the diagnosis and treatment of heart diseases relating to pressure overload, including but not limited to those which lead to heart failure. Among other pathologies, pressure overload induces the development of left ventricular hypertrophy (LVH) and left atrial enlargement (LAE) in the mammalian heart.

Specifically, genes are identified and described herein that are differentially expressed following induced pressure overload of the heart. The detection of the coding sequence and/or polypeptide products of these genes provides useful methods for early detection, diagnosis, staging, and monitoring of conditions leading to hypertrophy and enlargement of the heart, e.g. by the analysis of blood samples, biopsy material, in vivo imaging, metabolic assays for enzymatic activities, and the like. Expression signatures of a set of genes in heart tissue may also be evaluated for conditions indicative of pressure overload of the heart.

The invention also provides methods for the identification of compounds that modulate the expression of genes or the activity of gene products in heart diseases involving pressure overload, as well as methods for the treatment of disease by administering such compounds to individuals exhibiting heart failure symptoms or tendencies.

### BRIEF DESCRIPTION OF THE DRAWINGS

FIG. 1. Summary of data analysis. After background subtraction and dye bias normalization, poor quality features with low signal intensity were excluded from further analysis. Features with valid values in at least 66% of the experiments for each pairwise comparison (e.g., LA>66% AND TAC LA>66%) were retained for further analysis using SAM and t-test. Lists of genes identified as up-or downregulated by SAM were then mapped to GO terms and Fisher's exact test used to identify biological process groups with significant groupwide regulation.

FIG. 2. Hierarchical clustering. Left atria from TAC animals cluster more closely with ventricles than atria.

FIGS. 3A-3B. SAM analysis. Heatmaps of the top most significantly up- and downregulated genes in TAC LA(a) and LV(b). The order of the genes reflects decreasing SAM score, or d-statistic.

FIG. 4. Heatmap of the 891 upregulated and 1001 downregulated genes identified by SAM in the TAC LA. Blocks of genes with ventricle-like, atrial-like, and novel TAC expression patterns are highlighted. Red color denotes high expression, green denotes low expression level.

FIG. 5A-5C. Top statistically significantly regulated gene ontology biological process groups for TAC LA(a and b) and LV(c). The figure lists the biological process group, the total number of annotated genes in that group on the array, the number of genes identified by SAM as up- or downregulated in the group, and the one sided Fisher's exact p-value for differential regulation of each group.

FIG. 6. Energy pathway genes downregulated in TAC LA. This figure shows the breadth of downregulation of the TCA cycle, fatty acid metabolism, and oxidative phosphorylation genes that occur in response to pressure overload in the LA. Downregulated genes from each oxidative phosphorylation complex are listed in the graphic. A similar number of genes is downregulated in the TAC LV.

FIG. 7. Comparison of microarray and qRT-PCR results. Expression is plotted as log(10) fold expression change versus sham operated control for LA and LV tissues. This figure illustrates that fold changes in expression are usually greater in the LA than LV. Results are shown for the 9 regulated genes (frizzled-related protein (Frzb), cyclin D1, TGFβ2, HIF1a, endothelin receptor b (Ednrb), four-and-a-half LIM domains 2 (FHL2), regulator of G-protein signaling 2 (RGS2), diacylglycerol O-acyltransferase 2 (DGAT2), and homeodomain-only protein (Hop)) for which qRT-PCR validation was performed.

Table I pg. 1-pg. 26 provides a list of genetic sequences differentially expressed following transverse aortic constriction. The Stanford Gene ID refers to the internet address of genome-www5.stanford.edu, which provides a database including Genbank accession numbers. Pages 1-12 provide for significantly upregulated genes, and pages 13-26 provide for significantly down-regulated genes. Table IA pg. 1-pg. 3 provides a subset of upregulated genes of interest, and includes under the heading “UGRepAcc [A]” the accession numbers for representative genetic sequences available at Genbank. Under the heading “LLRepProtAcc [A]” are provided accession numbers for representative protein sequences at Genbank. Table IB provides a further subset of sequences of interest, similarly annotated. The sequences of Table IA or Table IB pg. 1-pg. 2 may be further sub-divided according to their representation in Tables II, III or IV.

Table II pg. 1-pg. 4 provides a list of genetic sequences set forth in Table I, which are differentially expressed following transverse aortic constriction, which are of interest for serologic assays. Table II further provides Genbank accession numbers, Genbank accession numbers of human homologs, and whether the gene is upregulated in transverse aortic constriction in the left atrium (designated UP TAC LA) and/or the left ventricle (designated UP TAC LV).

Table III pg. 1-pg. 4 provides a list of genetic sequences set forth in Table I, differentially expressed following transverse aortic constriction, which are of interest for imaging assays. Table III further provides Genbank accession numbers, Genbank accession numbers of human homologs, and whether the gene is upregulated in transverse aortic constriction in the left atrium (designated UP TAC LA) and/or the left ventricle (designated UP TAC LV).

Table IV pg 1-pg. 3 provides a list of genetic sequences set forth in Table I, differentially expressed following transverse aortic constriction, which are of interest for metabolic assays. Table IV further provides Genbank accession numbers, Genbank accession numbers of human homologs, and whether the gene is upregulated in transverse aortic constriction in the left atrium (designated UP TAC LA) and/or the left ventricle (designated UP TAC LV).

### DETAILED DESCRIPTION OF THE EMBODIMENTS

Methods and compositions for the diagnosis and treatment of heart diseases involving pressure overload, including but not limited to cardiomyopathies; heart failure; and the like, are provided. The invention is based, in part, on the evaluation of the expression and role of genes that are differentially expressed in response to pressure overload, e.g. during left atrial enlargement and left ventricular hypertrophy. The right chambers may have similar changes in gene expression in association with pathologies such as pulmonary hypertension, etc. Such sequences are useful in the diagnosis and monitoring of cardiac disease. The gene products are also useful as therapeutic targets for drug screening and action.

To systematically investigate the transcriptional changes that mediate these processes, a genome-wide transcriptional profiling of each of the four heart chambers was performed following transverse aortic constriction. It is shown herein that during enlargement, the left atrium undergoes radical changes in gene transcription. Structural changes in the LA and LV are correlated with significant changes in the transcriptional profile of these chambers. Statistical analysis of the results identified biological process groups with significant group-wide changes, including angiogenesis, fatty acid oxidation, oxidative phosphorylation, cytoskeletal and matrix reorganization, and G-protein coupled receptor signaling. The genes thus identified, and their classification into biological process groups, are provided in Table I. Subsets of the upregulated genes are provided in Tables IA and IB. Table IA is a subset of Table I, and Table IB is a subset of Table IA.

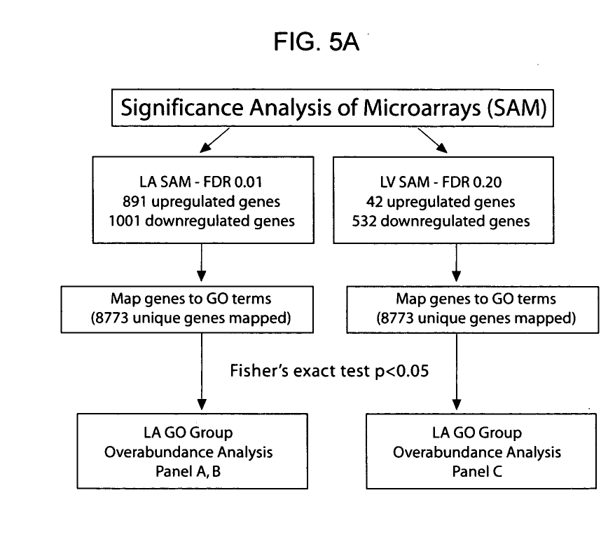

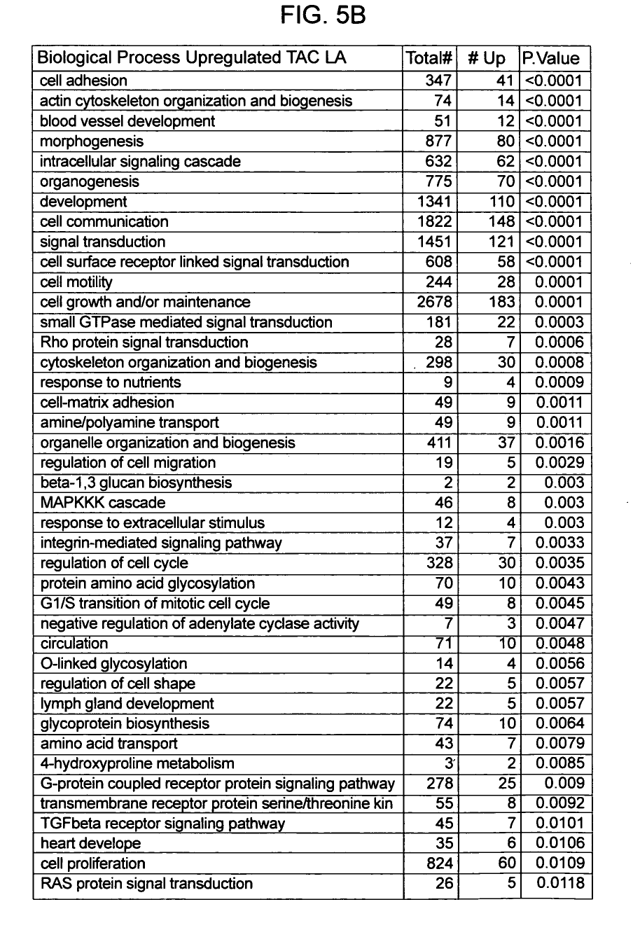

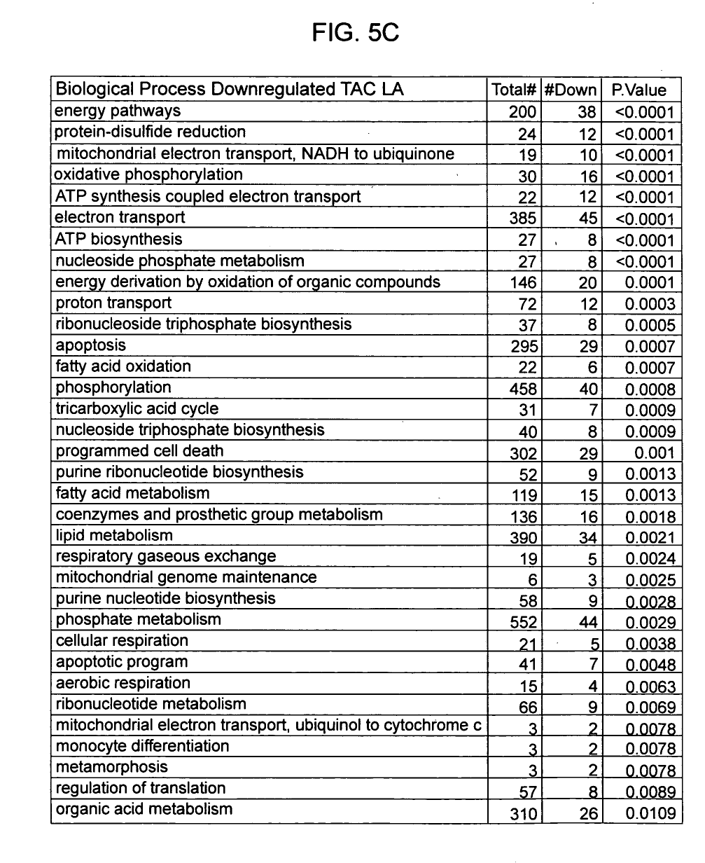

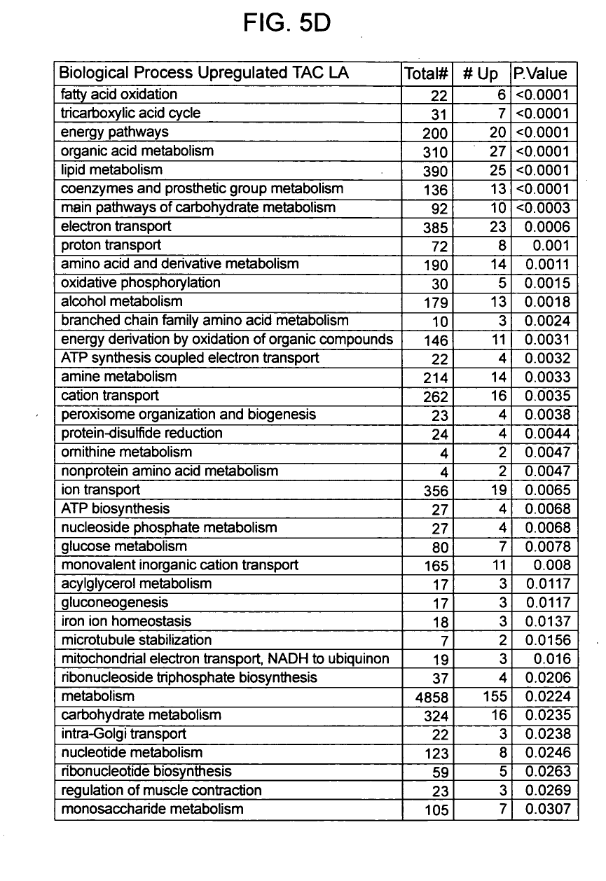

For some methods of the invention, a panel of sequences will be selected, comprising, for example, at least one, at least two, at least three, at least four, at least five, at least ten, at least 15, at least 20, and may include substantially all the sequences of a specific Table (I, IA, IB; and/or II, III, IV), or may be limited to not more than about 100 distinct sequences, not more than about 50 distinct sequences, not more than about 25 distinct sequences, and the like. The selection of sequences for inclusion in arrays, use in diagnostic panels, and the like may be based on representation of a sequence in one or more of the sub-tables, e.g. selecting sequences present in Table IA or Table IB; representation of a sequence in both Table IB and Table II; Table IB and Table III; Table IB and Table IV, and the like. The use of human homologs of the sequences is of particular interest. Selection of sequences may alternatively be based on a cut-off for significance or for fold-change in expression, e.g. those sequences have a fold-change of at least about 3, at least about 6, at least 10, or more. Selection of sequences may also be based on biological activity grouping, e.g. using the grouping as set forth in FIG. 5, genes can be divided into energy pathways, cell adhesion, cell communication, signal transduction, etc., where

The identification of pressure overload associated genes provides diagnostic and prognostic methods, which detect the occurrence of a disorder, e.g. cardiomyopathy; atrial enlargement; myocardial hypertrophy; etc., particularly where such a disorder is indicative of a propensity for heart failure; or assess an individual's susceptibility to such disease, by detecting altered expression of pressure overload associated genes. Early detection of genes or their products can be used to determine the occurrence of developing disease, thereby allowing for intervention with appropriate preventive or protective measures.

Various techniques and reagents find use in the diagnostic methods of the present invention. In one embodiment of the invention, blood samples, or samples derived from blood, e.g. plasma, serum, etc. are assayed for the presence of polypeptides encoded by pressure overload associated genes, e.g. cell surface and, of particular interest, secreted polypeptides. Such polypeptides may be detected through specific binding members. The use of antibodies for this purpose is of particular interest. Various formats find use for such assays, including antibody arrays; ELISA and RIA formats; binding of labeled antibodies in suspension/solution and detection by flow cytometry, mass spectroscopy, and the like. Detection may utilize one or a panel of antibodies. A subset of genes and gene products of interest for serologic assays are provided in Table II. These sequences may be further defined by reference to the sequences set forth in Table IA and/or Table IB, i.e. sequences that are present in both Table II, and Table IA or Table IB, may be of particular interest for serologic assays.

In another embodiment, in vivo imaging is utilized to detect the presence of pressure overload associated gene on heart tissue. Such methods may utilize, for example, labeled antibodies or ligands specific for cell surface pressure overload associated gene products. Included for such methods are gene products differentially expressed on chambers of the heart, which can be localized by in situ binding of a labeled reagent. In these embodiments, a detectably-labeled moiety, e.g., an antibody, ligand, etc., which is specific for the polypeptide is administered to an individual (e.g., by injection), and labeled cells are located using standard imaging techniques, including, but not limited to, magnetic resonance imaging, computed tomography scanning, and the like. Detection may utilize one or a cocktail of imaging reagents. A subset of genes and gene products of interest for imaging assays are provided in Table III. These sequences may be further defined by reference to the sequences set forth in Table IA and/or Table IB, i.e. sequences that are present in both Table III, and Table IA or Table IB, may be of particular interest for imaging assays.

In another embodiment, metabolic tests are performed, e.g. with a labeled substrate, to determine the level of enzymatic activity of a pressure overload associated gene product. Gene products of interest for such assays include enzymes whose reaction product is readily detected, e.g. in blood samples. It is shown herein, for example, that oxidative phosphorylation is markedly downregulated during left ventricular hypertrophy and atrial enlargement, and provides a marker for risk of heart failure. A subset of genes and gene products of interest for metabolic assays are provided in Table IV. These sequences may be further defined by reference to the sequences set forth in Table IA and/or Table IB, i.e. sequences that are present in both Table IV and Table IA or Table IB may be of particular interest for metabolic assays.

In another embodiment, an mRNA sample from heart tissue, preferably from one or more chambers affected by pressure overload, is analyzed for the genetic signature indicating pressure overload, and diagnostic of a tendency to heart failure. Expression signatures typically utilize a panel of genetic sequences, e.g. a microarray format; multiplex amplification, etc., coupled with analysis of the results to determine if there is a statistically significant match with a disease signature.

Functional modulation of pressure overload associated genes and their products provides a point of intervention to block the pathophysiologic processes of hypertrophy and enlargement, and also provides therapeutic intervention in other cardiovascular system diseases with similar pathophysiologies. These genes and their products can also be used to prevent, attenuate or reduce damage in prophylactic strategies in patients at high-risk of heart failure. Genes whose expression is altered during development of hypertrophy or enlargement may be cardiodamaging. Agent(s) that inhibit the activity or expression of cardiodamaging genes can be used as a therapeutic or prophylactic agent. The agent that acts to decrease such gene product activity can be an anti-sense or RNAi nucleic acid that includes a segment corresponding a cardiodamaging gene, or any agent that acts as a direct or indirect inhibitor of the gene product, e.g. a pharmacological agonist, or partial agonist.

### Disease Conditions

Heart failure is a general term that describes the final common pathway of many disease processes. Heart failure is usually caused by a reduction in the efficiency of cardiac muscle contraction. However, mechanical overload with normal or elevated cardiac contraction can also cause heart failure. This mechanical overload may be due to arterial hypertension, or stenosis or leakage of the aortic, mitral, or pulmonary valves, or other causes. The initial response to overload is usually hypertrophy (cellular enlargement) of the myocardium to increase force production, returning cardiac output (CO) to normal levels. Typically, a hypertrophic heart has impaired relaxation, a syndrome referred to as diastolic dysfunction. In the natural history of the disease, compensatory hypertrophy in the face of ongoing overload is followed by thinning, dilation, and enlargement, resulting in systolic dysfunction, also commonly known as heart failure. This natural progression typically occurs over the course of months to many years in humans, depending on the severity of the overload stimulus. Intervention at the hypertrophy stage can slow or prevent the progression to the clinically significant systolic dysfunction stage. Thus, diagnosis in the early hypertrophy stage provides unique therapeutic opportunities. The most common cause of congestive heart failure is coronary artery disease, which can cause a myocardial infarction (heart attack), which forces the heart to carry out the same work with fewer heart cells. The result is a pathophysiological state where the heart is unable to pump out enough blood to meet the nutrient and oxygen requirements of metabolizing tissues or cells.

in LV failure, CO declines and pulmonary venous pressure increases. Elevated pulmonary capillary pressure to levels that exceed the oncotic pressure of the plasma proteins (about 24 mm Hg) leads to increased lung water, reduced pulmonary compliance, and a rise in the O₂ cost of the work of breathing. Pulmonary venous hypertension and edema resulting from LV failure significantly alter pulmonary mechanics and, thereby, ventilation/perfusion relationships. When pulmonary venous hydrostatic pressure exceeds plasma protein oncotic pressure, fluid extravasates into the capillaries, the interstitial space, and the alveoli.

Increased heart rate and myocardial contractility, arteriolar constriction in selected vascular beds, venoconstriction, and Na and water retention compensate in the early stages for reduced ventricular performance. Adverse effects of these compensatory efforts include increased cardiac work, reduced coronary perfusion, increased cardiac preload and afterload, fluid retention resulting in congestion, myocyte loss, increased K excretion, and cardiac arrhythmia.

The mechanism by which an asymptomatic patient with cardiac dysfunction develops overt CHF is unknown, but it begins with renal retention of Na and water, secondary to decreased renal perfusion. Thus, as cardiac function deteriorates, renal blood flow decreases in proportion to the reduced CO, the GFR falls, and blood flow within the kidney is redistributed. The filtration fraction and filtered Na decrease, but tubular resorption increases.

Although symptoms and signs, for example exertional dyspnea, orthopnrea, edema, tachycardia, pulmonary rales, a third heart sound, jugular venous distention, etc. have a diagnostic specificity of 70 to 90%, the sensitivity and predictive accuracy of conventional tests are low. Elevated levels of B-type natriuretic peptide may be diagnostic. Adjunctive tests include CBC, blood creatinine, BUN, electrolytes (eg, Mg, Ca), glucose, albumin, and liver function tests. ECG may be performed in all patients with HF, although findings are not specific.

Patients diagnosed as being at risk for heart failure by the methods of the invention may be appropriately treated to reduce the risk of heart failure. Drug treatment of systolic dysfunction primarily involves diuretics, ACE inhibitors, digitalis, and β-blockers; most patients are treated with at least two of these classes. Addition of hydralazine and isosorbide dinitrate to standard triple therapy of HF may improve hemodynamics and exercise tolerance and reduce mortality in refractory patients. The angiotensin II receptor blocker losartan has effects similar to those of ACE inhibitors.

Digitalis preparations have many actions, including weak inotropism, and blockade of the atrioventricular node. Digoxin is the most commonly prescribed digitalis preparation. Digitoxin, an alternative in patients with known or suspected renal disease, is largely excreted in the bile and is thus not influenced by abnormal renal function.

With careful administration of β-blockers, some patients, especially those with idiopathic dilated cardiomyopathy, will improve clinically and may have reduced mortality. Carvedilol, a 3rd-generation nonselective β-blocker, is also a vasodilator with α blockade and an antioxidant activity. Vasodilators such as nitroglycerin or nitroprusside improve ventricular function by reducing systolic ventricular wall stress, aortic impedance, ventricular chamber size, and valvular regurgitation.

Arterial hypertension, or the elevation of systolic and/or diastolic BP, either primary or secondary, is frequently associated with pressure overload of the heart, and is an important risk factor for heart failure. Hypertensive patients may be analyzed by the diagnostic methods of the invention, in order to determine whether there is a concurrent development of hypertrophy, diastolic dysfunction, and a tendency to heart failure. Criteria for hypertension is typically over about 140 mm Hg systolic blood pressure, and/or diastolic blood pressure of greater than about 90 mm Hg.

Primary (essential) hypertension is of unknown etiology; its diverse hemodynamic and pathophysiologic derangements are unlikely to result from a single cause. Heredity is a predisposing factor, but the exact mechanism is unclear. The pathogenic mechanisms can lead to increased total peripheral vascular resistance by inducing vasoconstriction and to increased cardiac output.

While no early pathologic changes occur in primary hypertension, ultimately, generalized arteriolar sclerosis develops. Left ventricular hypertrophy and, eventually, dilation develop gradually. Coronary, cerebral, aortic, renal, and peripheral atherosclerosis are more common and more severe in hypertensives because hypertension accelerates atherogenesis.

Valvular disease, including stenosis or insufficiency of the aortic, mitral, pulmonary, or tricuspid valves, is also frequently associated with overload of the heart, and is another important risk factor for heart failure. Patients with valvular disease may be analyzed by the diagnostic methods of the invention, in order to determine whether other is a concurrent development of hypertrophy, diastolic dysfunction, and a tendency to heart failure. Valvular disease is typically diagnosed by echocardiographic measurement of significant valvular stenoses or insufficiencies. Valvular heart disease has many etiologies, including but not limited to rheumatic heart disease, congenital valve defects, endocarditis, aging, etc. The pathogenic mechanism whereby valvular disease leads to heart failure is the obstruction of blood outflow from various chambers of the heart, thus increasing load.

Cardiomyopathy refers to a structural or functional abnormality of the ventricular myocardium. Cardiomyopathy has many causes. Pathophysiologic classification (dilated congestive, hypertrophic, or restrictive cardiomyopathy) by means of history, physical examination, and invasive or noninvasive testing may be performed. If no cause can be found, cardiomyopathy is considered primary or idiopathic.

Dilated congestive cardiomyopathies include disorders of myocardial function with heart failure, in which ventricular dilation and systolic dysfunction predominate. The most common identifiable cause in temperate zones is diffuse coronary artery disease with diffuse ischemic myopathy. Most commonly, at presentation there is chronic myocardial fibrosis with diffuse loss of myocytes. Diagnosis depends on the characteristic history and physical examination and exclusion of other causes of ventricular failure. The ECG may show sinus tachycardia, low-voltage QRS, and nonspecific ST segment depression with low-voltage or inverted T waves.

Hypertrophic cardiomyopathies are congenital or acquired disorders characterized by marked ventricular hypertrophy with diastolic dysfunction that may develop in the absence of increased afterload. The cardiac muscle is abnormal with cellular and myofibrillar disarray, although this finding is not specific to hypertrophic cardiomyopathy. The interventricular septum may be hypertrophied more than the left ventricular posterior wall (asymmetric septal hypertrophy). In the most common asymmetric form of hypertrophic cardiomyopathy, there is marked hypertrophy and thickening of the upper interventricular septum below the aortic valve. During systole, the septum thickens and the anterior leaflet of the mitral valve, already abnormally oriented due to the abnormal shape of the ventricle, is sucked toward the septum, producing outflow tract obstruction. Clinical manifestations may occur alone or in any combination: Chest pain is usually typical angina related to exertion. Syncope is usually exertional and due to a combination of cardiomyopathy, arrhythmia, outflow tract obstruction, and poor diastolic filling of the ventricle. Dyspnea on exertion results from poor diastolic compliance of the left ventricle, which leads to a rapid rise in left ventricular end-diastolic pressure as flow increases. Outflow tract obstruction, by lowering cardiac output, may contribute to the dyspnea.

Restrictive cardiomyopathies are characterized by rigid, noncompliant ventricular walls that resist diastolic filling of one or both ventricles, most commonly the left. The cause is usually unknown. Amyloidosis involving the myocardium is usually systemic, as is iron infiltration in hemochromatosis. Sarcoidosis and Fabry's disease involve the myocardium, and nodal conduction tissue can be involved. Löffler's disease (a subcategory of hypereosinophilic syndrome with primary cardiac involvement) is a cause of restrictive cardiomyopathy. It occurs in the tropics. It begins as an acute arteritis with eosinophilia, with subsequent thrombus formation on the endocardium, chordae, and atrioventricular valves, progressing to fibrosis. Endocardial fibrosis occurs in temperate zones and involves only the left ventricle. The main hemodynamic consequence of these pathologic states is diastolic dysfunction with a rigid, noncompliant chamber with a high filling pressure. Systolic function may deteriorate if compensatory hypertrophy is inadequate in cases of infiltrated or fibrosed chambers. Mural thrombosis and systemic emboli can complicate the restrictive or obliterative variety.

### Identification of Genes Associated With Pressure Overload

In order to identify pressure overload associated genes, tissue was taken from the chambers of the heart following transverse aortic constriction, or from control, unaffected tissue. RNA, either total or mRNA, is isolated from such tissues. See, for example, Sambrook et al., 1989, Molecular Cloning, A Laboratory Manual, Cold Spring Harbor Press, New York; and Ausubel, F. M. et al., eds., 1987-1993, Current Protocols in Molecular Biology, John Wiley & Sons, Inc., New York, both of which are incorporated herein by reference in their entirety. Differentially expressed genes are detected by comparing gene expression levels between the experimental and control conditions. Transcripts within the collected RNA samples that represent differentially expressed genes may be identified by utilizing a variety of methods known to those of skill in the art, including differential screening, subtractive hybridization, differential display, or hybridization to an array comprising a plurality of gene sequences.

“Differential expression” as used herein refers to both quantitative as well as qualitative differences in the genes' temporal and/or tissue expression patterns. Thus, a differentially expressed gene may have its expression activated or inactivated in normal versus disease conditions, or in control versus experimental conditions. Preferably, a regulated gene will exhibit an expression pattern within a given tissue or cell type that is detectable in either control or disease subjects, but is not detectable in both. Detectable, as used herein, refers to an RNA expression pattern or presence of polypeptide product that is detectable via the standard techniques of differential display, reverse transcription-(RT-) PCR and/or Northern analyses, ELISA, RIA, metabolic assays, etc., which are well known to those of skill in the art. Generally, differential expression means that there is at least a 20% change, and in other instances at least a 2-, 3-, 5- or 10-fold difference between disease and control tissue expression. The difference usually is one that is statistically significant, meaning that the probability of the difference occurring by chance (the P-value) is less than some predetermined level (e.g., 5%). Usually the confidence level (P value) is <0.05, more typically <0.01, and in other instances, <0.001.

Table I provides a list of sequences that have significantly altered expression in hypertrophic cardiomyopathy, which genes may be induced or repressed as indicated in the table. Table IA provides a subset of upregulated genes of interest. Table IB provides a further subset of upregulated sequences of interest. The sequences of Table IA or Table IB may be further sub-divided according to their representation in Tables II, III or IV. In some embodiments, the sequences of interest have a “fold change” as set forth in Table I, of at least about 4; of a least about 5, of at least about 6, or more.

### Nucleic Acids

The sequences of pressure overload associated genes find use in diagnostic and prognostic methods, for the recombinant production of the encoded polypeptide, and the like. A list of pressure overload associated genetic sequences is provided in Table I, and in the sub-tables thereof. The nucleic acids of the invention include nucleic acids having a high degree of sequence similarity or sequence identity to one of the sequences provided in Table 1, and also include homologs, particularly human homologs, examples of which are provided in Tables II, III and IV. Sequence identity can be determined by hybridization under stringent conditions, for example, at 50° C. or higher and 0.1×SSC (9 mM NaCl/0.9 mM Na citrate). Hybridization methods and conditions are well known in the art, see, e.g., U.S. Pat. No. 5,707,829. Nucleic acids that are substantially identical to the provided nucleic acid sequence, e.g. allelic variants, genetically altered versions of the gene, etc., bind to one of the sequences provided in Table I and sub-tables thereof under stringent hybridization conditions. Further specific guidance regarding the preparation of nucleic acids is provided by Fleury et al. (1997) Nature Genetics 15:269-272; Tartaglia et al., PCT Publication No. WO 96/05861; and Chen et al., PCT Publication No. WO 00/06087, each of which is incorporated herein in its entirety.

The genes listed in Table I and sub-tables thereof may be obtained using various methods well known to those skilled in the art, including but not limited to the use of appropriate probes to detect the genes within an appropriate cDNA or genomic DNA library, antibody screening of expression libraries to detect cloned DNA fragments with shared structural features, direct chemical synthesis, and amplification protocols. Libraries are preferably prepared from nerve cells. Cloning methods are described in Berger and Kimmel, Guide to Molecular Cloning Techniques, Methods in Enzymology, 152, Academic Press, Inc. San Diego, Calif.; Sambrook, et al. (1989) Molecular Cloning—A Laboratory Manual (2nd ed) Vols. 1-3, Cold Spring Harbor Laboratory, Cold Spring Harbor Press, N.Y.; and Current Protocols (1994), a joint venture between Greene Publishing Associates, Inc. and John Wiley and Sons, Inc.

The sequence obtained from clones containing partial coding sequences or non-coding sequences can be used to obtain the entire coding region by using the RACE method (Chenchik et al. (1995) CLONTECHniques (X) 1: 5-8). Oligonucleotides can be designed based on the sequence obtained from the partial clone that can amplify a reverse transcribed mRNA encoding the entire coding sequence. Alternatively, probes can be used to screen cDNA libraries prepared from an appropriate cell or cell line in which the gene is transcribed. Once the target nucleic acid is identified, it can be isolated and cloned using well-known amplification techniques. Such techniques include the polymerase chain reaction (PCR) the ligase chain reaction (LCR), Qβ-replicase amplification, the self-sustained sequence replication system (SSR) and the transcription based amplification system (TAS). Such methods include, those described, for example, in U.S. Pat. No. 4,683,202 to Mullis et al.; PCR Protocols A Guide to Methods and Applications (Innis et al. eds) Academic Press Inc. San Diego, Calif. (1990); Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117.

As an alternative to cloning a nucleic acid, a suitable nucleic acid can be chemically synthesized. Direct chemical synthesis methods include, for example, the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Left., 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. While chemical synthesis of DNA is often limited to sequences of about 100 bases, longer sequences can be obtained by the ligation of shorter sequences. Alternatively, subsequences may be cloned and the appropriate subsequences cleaved using appropriate restriction enzymes.

The nucleic acids can be cDNAs or genomic DNAs, as well as fragments thereof. The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ or 3′ end of the transcribed region. The genomic DNA flanking the coding region, either 3′ or 5′, or internal regulatory sequences as sometimes found in introns, contains sequences required for proper tissue, stage-specific, or disease-state specific expression, and are useful for investigating the up-regulation of expression in tumor cells.

Probes specific to the nucleic acid of the invention can be generated using the nucleic acid sequence disclosed in Table I and sub-tables thereof. The probes are preferably at least about 18 nt, 25 nt, 50 nt or more of the corresponding contiguous sequence of one of the sequences provided in Table I and sub-tables thereof, and are usually less than about 2, 1, or 0.5 kb in length. Preferably, probes are designed based on a contiguous sequence that remains unmasked following application of a masking program for masking low complexity, e.g. BLASTX. Double or single stranded fragments can be obtained from the DNA sequence by chemically synthesizing oligonucleotides in accordance with conventional methods, by restriction enzyme digestion, by PCR amplification, etc. The probes can be labeled, for example, with a radioactive, biotinylated, or fluorescent tag.

The nucleic acids of the subject invention are isolated and obtained in substantial purity, generally as other than an intact chromosome. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome.

The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other. For hybridization probes, it may be desirable to use nucleic acid analogs, in order to improve the stability and binding affinity. The term “nucleic acid” shall be understood to encompass such analogs.

### Polypeptides

Polypeptides encoded by pressure overload associated genes are of interest for screening methods, as reagents to raise antibodies, as therapeutics, and the like. Such polypeptides can be produced through isolation from natural sources, recombinant methods and chemical synthesis. In addition, functionally equivalent polypeptides may find-use, where the equivalent polypeptide may be a homolog, e.g. a human homolog, may contain deletions, additions or substitutions of amino acid residues that result in a silent change, thus producing a functionally equivalent gene product. Amino acid substitutions may be made on the basis of similarity in polarity, charge, solubility, hydrophobicity, hydrophilicity, and/or the amphipathic nature of the residues involved. “Functionally equivalent”, as used herein, refers to a protein capable of exhibiting a substantially similar in vivo activity as the polypeptide encoded by an pressure overload associated gene, as provided in Table I and sub-tables thereof.

Peptide fragments find use in a variety of methods, where fragments are usually at least about 10 amino acids in length, about 20 amino acids in length, about 50 amino acids in length, or longer, up to substantially full length. Fragments of particular interest include fragments comprising an epitope, which can be used to raise specific antibodies. Soluble fragment of cell surface proteins are also of interest, e.g. truncated at transmembrane domains.

The polypeptides may be produced by recombinant DNA technology using techniques well known in the art. Methods that are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized.

Typically, the coding sequence is placed under the control of a promoter that is functional in the desired host cell to produce relatively large quantities of the gene product. An extremely wide variety of promoters are well-known, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Expression can be achieved in prokaryotic and eukaryotic cells utilizing promoters and other regulatory agents appropriate for the particular host cell. Exemplary host cells include, but are not limited to, E. coli, other bacterial hosts, yeast, and various higher eukaryotic cells such as the COS, CHO and HeLa cells lines and myeloma cell lines.

In mammalian host cells, a number of viral-based expression systems may be used, including retrovirus, lentivirus, adenovirus, adeno associated virus, and the like. In cases where an adenovirus is used as an expression vector, the coding sequence of interest can be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing differentially expressed or pathway gene protein in infected hosts.

Specific initiation signals may also be required for efficient translation of the genes. These signals include the ATG initiation codon and adjacent sequences. In cases where a complete gene, including its own initiation codon and adjacent sequences, is inserted into the appropriate expression vector, no additional translational control signals may be needed. However, in cases where only a portion of the gene coding sequence is inserted, exogenous translational control signals must be provided. These exogenous translational control signals and initiation codons can be of a variety of origins, both natural and synthetic. The efficiency of expression may be enhanced by the inclusion of appropriate transcription enhancer elements, transcription terminators, etc.

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, etc.

For long-term, high-yield production of recombinant proteins, stable expression is preferred. For example, cell lines that stably express the differentially expressed or pathway gene protein may be engineered. Rather than using expression vectors that contain viral origins of replication, host cells can be transformed with DNA controlled by appropriate expression control elements, and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines that express the target protein. Such engineered cell lines may be particularly useful in screening and evaluation of compounds that affect the endogenous activity of the differentially expressed or pathway gene protein. A number of selection systems may be used, including but not limited to the herpes simplex virus thymidine kinase, hypoxanthine-guanine phosphoribosyltransferase, and adenine phosphoribosyltransferase genes. Antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin.

The polypeptide may be labeled, either directly or indirectly. Any of a variety of suitable labeling systems may be used, including but not limited to, radioisotopes such as ¹²⁵I; enzyme labeling systems that generate a detectable calorimetric signal or light when exposed to substrate; and fluorescent labels. Indirect labeling involves the use of a protein, such as a labeled antibody, that specifically binds to the polypeptide of interest. Such antibodies include but are not limited to polyclonal, monoclonal, chimeric, single chain, Fab fragments and fragments produced by an Fab expression library.

Once expressed, the recombinant polypeptides can be purified according to standard procedures of the art, including ammonium sulfate precipitation, affinity columns, ion exchange and/or size exclusivity chromatography, gel electrophoresis and the like (see, generally, R. Scopes, Protein Purification, Springer—Verlag, N.Y. (1982), Deutscher, Methods in Enzymology Vol. 182: Guide to Protein Purification, Academic Press, Inc. N.Y. (1990)).

As an option to recombinant methods, polypeptides and oligopeptides can be chemically synthesized. Such methods typically include solid-state approaches, but can also utilize solution based chemistries and combinations or combinations of solid-state and solution approaches. Examples of solid-state methodologies for synthesizing proteins are described by Merrifield (1964) J. Am. Chem. Soc. 85:2149; and Houghton (1985) Proc. Natl. Acad. Sci., 82:5132. Fragments of a CARDIOPROTECTIVE protein can be synthesized and then joined together. Methods for conducting such reactions are described by Grant (1992) Synthetic Peptides: A User Guide, W.H. Freeman and Co., N.Y.; and in “Principles of Peptide Synthesis,” (Bodansky and Trost, ed.), Springer-Verlag, Inc. N.Y., (1993).

### Arrays

Arrays provide a high throughput technique that can assay a large number of polynucleotides or polypeptides in a sample. In one aspect of the invention, an array is constructed comprising one or more of the pressure overload associated genes, gene products, binding members specific for the gene product, etc., as set forth in Table I and sub-tables thereof, preferably comprising at least 4 distinct genes or gene products, at least about 8, at least 10, at least about 15, at least about 25, or more of these sequences, which array may further comprise other sequences known to be up- or down-regulated in heart tissue.

This technology can be used as a tool to test for differential expression. Arrays can be created by spotting polynucleotide probes, antibodies, polypeptides, etc. onto a substrate (e.g., glass, nitrocellulose, etc.) in a two-dimensional matrix or array having bound probes. The probes can be bound to the substrate by either covalent bonds or by non-specific interactions, such as hydrophobic interactions. Techniques for constructing arrays and methods of using these arrays are described in, for example, Schena et al. (1996) Proc Natl Acad Sci USA. 93(20):10614-9; Schena et al. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734.

The probes utilized in the arrays can be of varying types and can include, for example, synthesized probes of relatively short length (e.g., a 20-mer or a 25-mer), cDNA (full length or fragments of gene), amplified DNA, fragments of DNA (generated by restriction enzymes, for example), reverse transcribed DNA, peptides, proteins, antibodies or fragments thereof, and the like. Arrays can be utilized in detecting differential expression levels.

Arrays can be used to, for example, examine differential expression of genes. For example, arrays can be used to detect differential expression of pressure overload associated genes, where expression is compared between a test cell and control cell. Exemplary uses of arrays are further described in, for example, Pappalarado et al. (1998) Sem. Radiation Oncol. 8:217; and Ramsay. (1998) Nature Biotechnol. 16:40. Furthermore, many variations on methods of detection using arrays are well within the skill in the art and within the scope of the present invention. For example, rather than immobilizing the probe to a solid support, the test sample can be immobilized on a solid support which is then contacted with the probe. Additional discussion regarding the use of microarrays in expression analysis can be found, for example, in Duggan, et al., Nature Genetics Supplement 21:10-14 (1999); Bowtell, Nature Genetics Supplement 21:25-32 (1999); Brown and Botstein, Nature Genetics Supplement 21:33-37 (1999); Cole et al., Nature Genetics Supplement 21:38-41 (1999); Debouck and Goodfellow, Nature Genetics Supplement 21:48-50 (1999); Bassett, Jr., et al., Nature Genetics Supplement 21:51-55 (1999); and Chakravarti, Nature Genetics Supplement 21:56-60 (1999).

For detecting expression levels, usually nucleic acids are obtained from a test sample, and either directly labeled, or reversed transcribed into labeled cDNA. Alternatively, a protein sample, e.g. a serum sample, may be used, and labeled following binding to the array. The test sample containing the nucleic acids or proteins is then contacted with the array. After allowing a period sufficient for any nucleic acid or protein present in the sample to bind to the probes, the array is typically subjected to one or more washes to remove unbound sample and to minimize nonspecific binding to the probes of the arrays. Binding of labeled sequences is detected using any of a variety of commercially available scanners and accompanying software programs.

For example, if the nucleic acids from the sample are labeled with fluorescent labels, hybridization intensity can be determined by, for example, a scanning confocal microscope in photon counting mode. Appropriate scanning devices are described by e.g., U.S. Pat. No. 5,578,832 to Trulson et al., and U.S. Pat. No. 5,631,734 to Stern et al. and are available from Affymetrix, Inc., under the GeneChip™ label. Some types of label provide a signal that can be amplified by enzymatic methods (see Broude, et al., Proc. Natl. Acad. Sci. U.S.A. 91, 3072-3076 (1994)). A variety of other labels are also suitable including, for example, radioisotopes, chromophores, magnetic particles and electron dense particles.

Those locations on the probe array that are bound to sample are detected using a reader, such as described by U.S. Pat. No. 5,143,854, WO 90/15070, and U.S. Pat. No. 5,578,832. For customized arrays, the hybridization pattern can then be analyzed to determine the presence and/or relative amounts or absolute amounts of known species in samples being analyzed as described in e.g., WO 97/10365.

### Specific Binding Members

The term “specific binding member” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). The complementary members of a specific binding pair are sometimes referred to as a ligand and receptor; or receptor and counter-receptor. For the purposes of the present invention, the two binding members may be known to associate with each other, for example where an assay is directed at detecting compounds that interfere with the association of a known binding pair. Alternatively, candidate compounds suspected of being a binding partner to a compound of interest may be used.

Specific binding pairs of interest include carbohydrates and lectins; complementary nucleotide sequences; peptide ligands and receptor; effector and receptor molecules; hormones and hormone binding protein; enzyme cofactors and enzymes; enzyme inhibitors and enzymes; lipid and lipid-binding protein; etc. The specific binding pairs may include analogs, derivatives and fragments of the original specific binding member. For example, a receptor and ligand pair may include peptide fragments, chemically synthesized peptidomimetics, labeled protein, derivatized protein, etc.

In a preferred embodiment, the specific binding member is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be polyclonal antibodies, although monoclonal antibodies are preferred because they may be reproduced by cell culture or recombinantly, and can be modified to reduce their antigenicity.

Polyclonal antibodies can be raised by a standard protocol by injecting a production animal with an antigenic composition, formulated as described above. See, e.g., Harlow and Lane, Antibodies: A Laboratory Manual, Cold Spring Harbor Laboratory, 1988. In one such technique, an antigen comprising an antigenic portion of the protein target is initially injected into any of a wide variety of mammals (e.g., mice, rats, rabbits, sheep or goats). When utilizing an entire protein, or a larger section of the protein, antibodies may be raised by immunizing the production animal with the protein and a suitable adjuvant (e.g., Freund's, Freund's complete, oil-in-water emulsions, etc.) When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the protein target, a superior immune response may be elicited if the polypeptide is joined to a carrier protein, such as ovalbumin, BSA or KLH. The peptide-conjugate is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. The immortal cell line utilized is preferably selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

Preferably, the immortal fusion partners utilized are derived from a line that does not secrete immunoglobulin. The resulting fused cells, or hybridomas, are cultured under conditions that allow for the survival of fused, but not unfused, cells and the resulting colonies screened for the production of the desired monoclonal antibodies. Colonies producing such antibodies are cloned, expanded, and grown so as to produce large quantities of antibody, see Kohler and Milstein, 1975 Nature 256:495 (the disclosures of which are hereby incorporated by reference).

Large quantities of monoclonal antibodies from the secreting hybridomas may then be produced by injecting the clones into the peritoneal cavity of mice and harvesting the ascites fluid therefrom. The mice, preferably primed with pristane, or some other tumor-promoter, and immunosuppressed chemically or by irradiation, may be any of various suitable strains known to those in the art. The ascites fluid is harvested from the mice and the monoclonal antibody purified therefrom, for example, by CM Sepharose column or other chromatographic means. Alternatively, the hybridomas may be cultured in vitro or as suspension cultures. Batch, continuous culture, or other suitable culture processes may be utilized. Monoclonal antibodies are then recovered from the culture medium or supernatant.

Monoclonal antibodies against the protein targets of the invention may be currently available from commercial sources. These antibodies are suitable for use in the compositions of the present invention.

In addition, the antibodies or antigen binding fragments may be produced by genetic engineering. In this technique, as with the standard hybridoma procedure, antibody-producing cells are sensitized to the desired antigen or immunogen. The messenger RNA isolated from the immune spleen cells or hybridomas is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell.). When antibody gene synthesis is induced in the transfected host, the heavy and light chain proteins self-assemble to produce active antibodies that can be detected by screening with the antigen or immunogen.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ficin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

In addition, derivatized immunoglobulins with added chemical linkers, detectable moieties, such as fluorescent dyes, enzymes, substrates, chemiluminescent moieties and the like, or specific binding moieties, such as streptavidin, avidin, or biotin, and the like may be utilized in the methods and compositions of the present invention. For convenience, the term “antibody” or “antibody moiety” will be used throughout to generally refer to molecules which specifically bind to an epitope of the protein targets, although the term will encompass all immunoglobulins, derivatives, fragments, recombinant or engineered immunoglobulins, and modified immunoglobulins, as described above.

### Diagnostic and Prognostic Methods

The differential expression of pressure overload associated genes indicates that these sequences can serve as markers for diagnosis, and in prognostic evaluations to detect individuals at risk for cardiac pathologies, including atrial enlargement, ventricular hypertrophy, heart failure, etc. Prognostic methods can also be utilized to monitor an individual's health status prior to and after an episode, as well as in the assessment of the severity of the episode and the likelihood and extent of recovery.

In general, such diagnostic and prognostic methods involve detecting an altered level of expression of pressure overload associated genes or gene products in the cells or tissue of an individual or a sample therefrom, to generate an expression profile. A variety of different assays can be utilized to detect an increase in pressure overload associated gene expression, including both methods that detect gene transcript and protein levels. More specifically, the diagnostic and prognostic methods disclosed herein involve obtaining a sample from an individual and determining at least qualitatively, and preferably quantitatively, the level of a pressure overload associated genes product expression in the sample. Usually this determined value or test value is compared against some type of reference or baseline value.

The term expression profile is used broadly to include a genomic expression profile, e.g., an expression profile of mRNAs, or a proteomic expression profile, e.g., an expression profile of one or more different proteins. Profiles may be generated by any convenient means for determining differential gene expression between two samples, e.g. quantitative hybridization of mRNA, labeled mRNA, amplified mRNA, cRNA, etc., quantitative PCR, ELISA for protein quantitation, and the like.

The expression profile may be generated from a biological sample using any convenient protocol. While a variety of different manners of generating expression profiles are known, such as those employed in the field of differential gene expression analysis, one representative and convenient type of protocol for generating expression profiles is array based gene expression profile generation protocols. Following obtainment of the expression profile from the sample being assayed, the expression profile is compared with a reference or control profile to make a diagnosis regarding the susceptibility phenotype of the cell or tissue from which the sample was obtained/derived. Typically a comparison is made with a set of cells from an unaffected, normal source. Additionally, a reference or control profile may be a profile that is obtained from a cell/tissue known to be predisposed to heart failure, and therefore may be a positive reference or control profile.

In certain embodiments, the obtained expression profile is compared to a single reference/control profile to obtain information regarding the phenotype of the cell/tissue being assayed. In yet other embodiments, the obtained expression profile is compared to two or more different reference/control profiles to obtain more in depth information regarding the phenotype of the assayed cell/tissue. For example, the obtained expression profile may be compared to a positive and negative reference profile to obtain confirmed information regarding whether the cell/tissue has the phenotype of interest.

The difference values, i.e. the difference in expression in the presence and absence of radiation may be performed using any convenient methodology, where a variety of methodologies are known to those of skill in the array art, e.g., by comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above. A statistical analysis step is then performed to obtain the weighted contribution of the set of predictive genes.

In one embodiment of the invention, blood samples, or samples derived from blood, e.g. plasma, serum, etc. are assayed for the presence of polypeptides encoded by pressure overload associated genes, e.g. cell surface and, of particular interest, secreted polypeptides. Such polypeptides may be detected through specific binding members. The use of antibodies for this purpose is of particular interest. Various formats find use for such assays, including antibody arrays; ELISA and RIA formats; binding of labeled antibodies in suspension/solution and detection by flow cytometry, mass spectroscopy, and the like. Detection may utilize one or a panel of specific binding members, e.g. specific for at least about 2, at least about 3, at least about 5, at least about 10 or more different gene products. A subset of genes and gene products of interest for serologic assays are provided in Table II.

In another embodiment, in vivo imaging is utilized to detect the presence of pressure overload associated gene on heart tissue. Such methods may utilize, for example, labeled antibodies or ligands specific for cell surface pressure overload associated gene products. Included for such methods are gene products differentially expressed on chambers of the heart, which can be localized by in situ binding of a labeled reagent. In these embodiments, a detectably-labeled moiety, e.g., an antibody, ligand, etc., which is specific for the polypeptide is administered to an individual (e.g., by injection), and labeled cells are located using standard imaging techniques, including, but not limited to, magnetic resonance imaging, computed tomography scanning, and the like. Detection may utilize one or a cocktail of imaging reagents e.g. imaging reagents specific for at least about 2, at least about 3, at least about 5, at least about 10 or more different gene products. A subset of genes and gene products of interest for imaging assays are provided in Table III.

In another embodiment, metabolic tests are performed, e.g. with a labeled substrate, to determine the level of enzymatic activity of a pressure overload associated gene product. Gene products of interest for such assays include enzymes whose reaction product is readily detected, e.g. in blood samples. It is shown herein, for example, that oxidative phosphorylation is markedly downregulated during atrial enlargement, and provides a marker for risk of heart failure. A subset of genes and gene products of interest for metabolic assays are provided in Table IV. Assays may be directed to one or more metabolic activities

In another embodiment, an mRNA sample from heart tissue, preferably from one or more chambers affected by pressure overload, is analyzed for the genetic signature indicating pressure overload, and diagnostic of a tendency to heart failure. Expression signatures typically utilize a panel of genetic sequences, e.g. a microarray format; multiplex amplification, etc., coupled with analysis of the results to determine if there is a statistically significant match with a disease signature.

Nucleic acids or binding members such as antibodies that are specific for polypeptides derived from the sequence of one of the sequences provided in Table I and sub-tables thereof can be used to screen patient samples for increased expression of the corresponding mRNA or protein. Samples can be obtained from a variety of sources. For example, since the methods are designed primarily to diagnosis and assess risk factors for humans, samples are typically obtained from a human subject. However, the methods can also be utilized with samples obtained from various other mammals, such as primates, e.g. apes and chimpanzees, mice, cats, rats, and other animals. Such samples are referred to as a patient sample.

Samples can be obtained from the tissues or fluids of an individual, as well as from cell cultures or tissue homogenates. For example, samples can be obtained from whole blood, heart tissue biopsy, serum, saliva, tears, urine, fecal material, sweat, buccal, skin, etc. Also included in the term are derivatives and fractions of such cells and fluids. Where cells are analyzed, the number of cells in a sample will often be at least about 10², usually at least 10³ and may be about 10⁴ or more. The cells may be dissociated, in the case of solid tissues, or tissue sections may be analyzed. Alternatively a lysate of the cells may be prepared.

Diagnostic samples are collected any time after an individual is suspected to have cardiomyopathy, atrial enlargement, ventricular hypertrophy, etc. or has exhibited symptoms that predict such pathologies. In prophylactic testing, samples can be obtained from an individual who present with risk factors that indicate a susceptibility to heart failure, which risk factors include high blood pressure, obesity, diabetes, etc. as part of a routine assessment of the individual's health status.

The various test values determined for a sample from an individual believed to suffer pressure overload, cardiac hypertrophy, diastolic dysfunction, and/or, a tendency to heart failure typically are compared against a baseline value to assess the extent of increased or decreased expression, if any. This baseline value can be any of a number of different values: In some instances, the baseline value is a value established in a trial using a healthy cell or tissue sample that is run in parallel with the test sample. Alternatively, the baseline value can be a statistical value (e.g., a mean or average) established from a population of control cells or individuals. For example, the baseline value can be a value or range that is characteristic of a control individual or control population. For instance, the baseline value can be a statistical value or range that is reflective of expression levels for the general population, or more specifically, healthy individuals not susceptible to stroke. Individuals not susceptible to stroke generally refer to those having no apparent risk factors correlated with heart failure, such as high blood pressure, high cholesterol levels, diabetes, smoking and high salt diet, for example.

Nucleic Acid Screening Methods

Some of the diagnostic and prognostic methods that involve the detection of a pressure overload associated gene transcript begin with the lysis of cells and subsequent purification of nucleic acids from other cellular material, particularly mRNA transcripts. A nucleic acid derived from an mRNA transcript refers to a nucleic acid for whose synthesis the mRNA transcript, or a subsequence thereof, has ultimately served as a template. Thus, a cDNA reverse transcribed from an mRNA, an RNA transcribed from that cDNA, a DNA amplified from the cDNA, an RNA transcribed from the amplified DNA, are all derived from the mRNA transcript and detection of such derived products is indicative of the presence and/or abundance of the original transcript in a sample. Thus, suitable samples include, but are not limited to, mRNA transcripts of pressure overload associated genes, cDNA reverse transcribed from the mRNA, cRNA transcribed from the cDNA, DNA amplified from pressure overload associated nucleic acids, and RNA transcribed from amplified DNA.

A number of methods are available for analyzing nucleic acids for the presence of a specific sequence, e.g. upregulated expression. The nucleic acid may be amplified by conventional techniques, such as the polymerase chain reaction (PCR), to provide sufficient amounts for analysis. The use of the polymerase chain reaction is described in Saiki et al. (1985) Science 239:487, and a review of techniques may be found in Sambrook, et al. Molecular Cloning: A Laboratory Manual, CSH Press 1989, pp. 14.2-14.33.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein(6-FAM),2,7-dimethoxy4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N,N,N-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified, labeled, cloned fragment, etc. is analyzed by one of a number of methods known in the art. Probes may be hybridized to northern or dot blots, or liquid hybridization reactions performed. The nucleic acid may be sequenced by dideoxy or other methods, and the sequence of bases compared to a wild-type sequence. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Fractionation is performed by gel or capillary electrophoresis, particularly acrylamide or agarose gels.

In situ hybridization methods are hybridization methods in which the cells are not lysed prior to hybridization. Because the method is performed in situ, it has the advantage that it is not necessary to prepare RNA from the cells. The method usually involves initially fixing test cells to a support (e.g., the walls of a microtiter well) and then permeabilizing the cells with an appropriate permeabilizing solution. A solution containing labeled probes for a pressure overload associated gene is then contacted with the cells and the probes allowed to hybridize with the nucleic acids. Excess probe is digested, washed away and the amount of hybridized probe measured. This approach is described in greater detail by Harris, D. W. (1996) Anal. Biochem. 243:249-256; Singer, et al. (1986) Biotechniques 4:230-250; Haase et al. (1984) Methods in Virology, vol. VII, pp. 189-226; and Nucleic Acid Hybridization: A Practical Approach (Hames, et al., eds., 1987).

A variety of so-called “real time amplification” methods or “real time quantitative PCR” methods can also be utilized to determine the quantity of pressure overload associated gene mRNA present in a sample. Such methods involve measuring the amount of amplification product formed during an amplification process. Fluorogenic nuclease assays are one specific example of a real time quantitation method that can be used to detect and quantitate pressure overload associated gene transcripts. In general such assays continuously measure PCR product accumulation using a dual-labeled fluorogenic oligonucleotide probe—an approach frequently referred to in the literature simply as the “TaqMan” method.

The probe used in such assays is typically a short (ca. 20-25 bases) polynucleotide that is labeled with two different fluorescent dyes. The 5′ terminus of the probe is typically attached to a reporter dye and the 3′ terminus is attached to a quenching dye, although the dyes can be attached at other locations on the probe as well. For measuring a pressure overload associated gene transcript, the probe is designed to have at least substantial sequence complementarity with a probe binding site on a pressure overload associated gene transcript. Upstream and downstream PCR primers that bind to regions that flank the pressure overload associated gene are also added to the reaction mixture.

When the probe is intact, energy transfer between the two fluorophors occurs and the quencher quenches emission from the reporter. During the extension phase of PCR, the probe is cleaved by the 5′ nuclease activity of a nucleic acid polymerase such as Taq polymerase, thereby releasing the reporter dye from the polynucleotide-quencher complex and resulting in an increase of reporter emission intensity that can be measured by an appropriate detection system.

One detector which is specifically adapted for measuring fluorescence emissions such as those created during a fluorogenic assay is the ABI 7700 manufactured by Applied Biosystems, Inc. in Foster City, Calif. Computer software provided with the instrument is capable of recording the fluorescence intensity of reporter and quencher over the course of the amplification. These recorded values can then be used to calculate the increase in normalized reporter emission intensity on a continuous basis and ultimately quantify the amount of the mRNA being amplified.

Additional details regarding the theory and operation of fluorogenic methods for making real time determinations of the concentration of amplification products are described, for example, in U.S. Pat. No. 5,210,015 to Gelfand, U.S. Pat. No. 5,538,848 to Livak, et al., and U.S. Pat. No. 5,863,736 to Haaland, as well as Heid, C. A., et al., Genome Research, 6:986-994 (1996); Gibson, U. E. M, et al., Genome Research 6:995-1001 (1996); Holland, P. M., et al., Proc. Natl. Acad. Sci. USA 88:7276-7280, (1991); and Livak, K. J., et al., PCR Methods and Applications 357-362 (1995), each of which is incorporated by reference in its entirety.

Polypeptide Screening Methods

Screening for expression of the subject sequences may be based on the functional or antigenic characteristics of the protein. Various immunoassays designed to quantitate proteins encoded by the sequences corresponding to the sequences provided in Table I and sub-tables thereof may be used in screening. Functional, or metabolic, protein assays have proven to be effective screening tools. The activity of the encoded protein in oxidative phosphorylation assays, etc., may be determined by comparison with unaffected individuals.

Detection may utilize staining of cells or histological sections, performed in accordance with conventional methods, using antibodies or other specific binding members that specifically bind to the pressure overload associated polypeptides. The antibodies or other specific binding members of interest, e.g. receptor ligands, are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and the polypeptide corresponding to a sequence of Table I and sub-tables thereof in a blood sample, cell lysate, etc. Measuring the concentration of the target protein in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assayable supports (for example, separate wells of a micromiter plate) containing antibodies. Preferably, a series of standards, containing known concentrations of the test protein is assayed in parallel with the samples or aliquots thereof to serve as controls. Preferably, each sample and standard will be added to multiple wells so that mean values can be obtained for each. The incubation time should be sufficient for binding, generally, from about 0.1 to 3 hr is sufficient. After incubation, the insoluble support is generally washed of non-bound components. Generally, a dilute non-ionic detergent medium at an appropriate pH, generally 7-8, is used as a wash medium. From one to six washes may be employed, with sufficient volume to thoroughly wash non-specifically bound proteins present in the sample.

After washing, a solution containing a second antibody is applied. The antibody will bind to one of the proteins of interest with sufficient specificity such that it can be distinguished from other components present. The second antibodies may be labeled to facilitate direct, or indirect quantification of binding. Examples of labels that permit direct measurement of second receptor binding include radiolabels, such as ³H or ¹²⁵I, fluorescers, dyes, beads, chemiluminescers, colloidal particles, and the like. Examples of labels that permit indirect measurement of binding include enzymes where the substrate may provide for a colored or fluorescent product. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules. Generally, from about 0.1 to 3 hr is sufficient, usually 1 hr sufficing.

After the second binding step, the insoluble support is again washed free of non-specifically bound material, leaving the specific complex formed between the target protein and the specific binding member. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for the pressure overload associated polypeptide as desired, conveniently using a labeling method as described for the sandwich assay.

In some cases, a competitive assay will be used. In addition to the patient sample, a competitor to the targeted protein is added to the reaction mix. The competitor and the pressure overload associated polypeptide compete for binding to the specific binding partner. Usually, the competitor molecule will be labeled and detected as previously described, where the amount of competitor binding will be proportional to the amount of target protein present. The concentration of competitor molecule will be from about 10 times the maximum anticipated protein concentration to about equal concentration in order to make the most sensitive and linear range of detection.

The detection methods can be provided as part of a kit. Thus, the invention further provides kits for detecting the presence of an mRNA corresponding to a sequence of Table I, II, or III, and/or a polypeptide encoded thereby, in a biological sample. Procedures using these kits can be performed by clinical laboratories, experimental laboratories, medical practitioners, or private individuals. The kits of the invention for detecting a polypeptide comprise a moiety that specifically binds the polypeptide, which may be a specific antibody. The kits of the invention for detecting a nucleic acid comprise a moiety that specifically hybridizes to such a nucleic acid. The kit may optionally provide additional components that are useful in the procedure, including, but not limited to, buffers, developing reagents, labels, reacting surfaces, means for detection, control samples, standards, instructions, and interpretive information.

Imaging In Vivo

In some embodiments, the methods are adapted for imaging use in vivo, e.g., to locate or identify sites where pressure overload associated genes are expressed. In these embodiments, a detectably-labeled moiety, e.g., an antibody, which is specific for the pressure overload associated polypeptide is administered to an individual (e.g., by injection), and labeled cells are located using standard imaging techniques, including, but not limited to, magnetic resonance imaging, computed tomography scanning, and the like.

For diagnostic in vivo imaging, the type of detection instrument available is a major factor in selecting a given radionuclide. The radionuclide chosen must have a type of decay that is detectable by a given type of instrument. In general, any conventional method for visualizing diagnostic imaging can be utilized in accordance with this invention. Another important factor in selecting a radionuclide for in vivo diagnosis is that its half-life be long enough that it is'still detectable at the time of maximum uptake by the target tissue, but short enough that deleterious radiation of the host is minimized. A currently used method for labeling with ^{99m}Tc is the reduction of pertechnetate ion in the presence of a chelating precursor to form the labile ^{99m}Tc-precursor complex, which, in turn, reacts with the metal binding group of a bifunctionally modified chemotactic peptide to form a ^{99m}Tc-chemotactic peptide conjugate.

The detectably labeled antibody is used in conjunction with imaging techniques, in order to analyze the expression of the target. In one embodiment, the imaging method is one of PET or SPECT, which are imaging techniques in which a radionuclide is synthetically or locally administered to a patient. The subsequent uptake of the radiotracer is measured over time and used to obtain information about the targeted tissue. Because of the high-energy (γ-ray) emissions of the specific isotopes employed and the sensitivity and sophistication of the instruments used to detect them, the two-dimensional distribution of radioactivity may be inferred from outside of the body.

Among the most commonly used positron-emitting nuclides in PET are included ¹¹C, ¹³N, ¹⁵O, and ¹⁸F. Isotopes that decay by electron capture and/or y emission are used in SPECT, and include ¹²³I and ^{99m}Tc.

Time Course Analyses

Certain prognostic methods of assessing a patient's risk of heart failure involve monitoring expression levels for a patient susceptible to heart failure, to track whether there is a change in expression of a pressure overload associated gene over time. An increase in expression over time can indicate that the individual is at increased risk for heart failure. As with other measures, the expression level for the patient at risk for heart failure is compared against a baseline value. The baseline in such analyses can be a prior value determined for the same individual or a statistical value (e.g., mean or average) determined for a control group (e.g., a population of individuals with no apparent neurological risk factors). An individual showing a statistically significant increase in pressure overload associated expression levels over time can prompt the individual's physician to take prophylactic measures to lessen the individual's potential for heart failure. For example, the physician can recommend certain life style changes (e.g., medication, improved diet, exercise program) to reduce the risk of heart failure.

### Databases of Expression Profiles

Also provided are databases of expression profiles of phenotype determinative genes. Such databases will typically comprise expression profiles of various cells/tissues having susceptible phenotypes, negative expression profiles, etc., where such profiles are further described below.

The expression profiles and databases thereof may be provided in a variety of media to facilitate their use. “Media” refers to a manufacture that contains the expression profile information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. Such presentation provides a skilled artisan with a ranking of similarities and identifies the degree of similarity contained in the test expression profile.

### Therapeutic/Prophylactic Treatment Methods

Agents that modulate activity of pressure overload associated genes provide a point of therapeutic or prophylactic intervention. Numerous agents are useful in modulating this activity, including agents that directly modulate expression, e.g. expression vectors, antisense specific for the targeted gene; and agents that act on the protein, e.g. specific antibodies and analogs thereof, small organic molecules that block catalytic activity, etc.

The genes, gene fragments, or the encoded protein or protein fragments are useful in therapy to treat disorders associated with defects in expression. From a therapeutic point of view, modulating activity may have a therapeutic effect on a number of degenerative disorders. For example, expression can be upregulated by introduction of an expression vector, enhancing expression, providing molecules that mimic the activity of the targeted polypeptide, etc.

Antisense molecules can be used to down-regulate expression in cells. The antisense reagent may be antisense oligonucleotides (ODN), particularly synthetic ODN having chemical modifications from native nucleic acids, or nucleic acid constructs that express such antisense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

In one embodiment of the invention, RNAi technology is used. As used herein, RNAi technology refers to a process in which double-stranded RNA is introduced into cells expressing a candidate gene to inhibit expression of the candidate gene, i.e., to “silence” its expression. The dsRNA is selected to have substantial identity with the candidate gene. In general such methods initially involve transcribing a nucleic acids containing all or part of a candidate gene into single- or double-stranded RNA. Sense and anti-sense RNA strands are allowed to anneal under appropriate conditions to form dsRNA. The resulting dsRNA is introduced into cells via various methods. Usually the dsRNA consists of two separate complementary RNA strands. However, in some instances, the dsRNA may be formed by a single strand of RNA that is self-complementary, such that the strand loops back upon itself to form a hairpin loop. Regardless of form, RNA duplex formation can occur inside or outside of a cell.

dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhaltacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

A number of options can be utilized to deliver the dsRNA into a cell or population of cells. For instance, RNA can be directly introduced intracellularly. Various physical methods are generally utilized in such instances, such as administration by microinjection (see, e.g., Zernicka-Goetz, et al. (1997) Development 124:1133-1137; and Wianny, et al. (1998) Chromosoma 107: 430-439). Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct.

### Compound Screening

Compound screening may be performed using an in vitro model, a genetically altered cell or animal, or purified protein corresponding to any one of the provided pressure overload associated genes. One can identify ligands or substrates that bind to, inhibit, modulate or mimic the action of the encoded polypeptide.

The polypeptides include those encoded by the provided genetic sequences, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or, where the polypeptide is a member of a protein family, a region associated with a consensus sequence). Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 500 aa in length, where the fragment will have a contiguous stretch of amino acids that is identical to a polypeptide encoded by a pressure overload associated gene, or a homolog thereof.

Transgenic animals or cells derived therefrom are also used in compound screening. Transgenic animals may be made through homologous recombination, where the normal locus corresponding to a pressure overload associated gene is altered. Alternatively, a nucleic acid construct is randomly integrated into the genome. Vectors for stable integration include plasmids, retroviruses and other animal viruses, YACs, and the like. A series of small deletions and/or substitutions may be made in the coding sequence to determine the role of different domains. Of interest is the use of pressure overload associated genes to construct transgenic animal models for heart failure. Specific constructs of interest include antisense sequences that block expression of the targeted gene and expression of dominant negative mutations. A detectable marker, such as lac Z may be introduced into the locus of interest, where up-regulation of expression will result in an easily detected change in phenotype. One may also provide for expression of the target gene or variants thereof in cells or tissues where it is not normally expressed or at abnormal times of development. By providing expression of the target protein in cells in which it is not normally produced, one can induce changes in cell behavior.

Compound screening identifies agents that modulate function of the pressure overload associated gene. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains.

The term “agent” as used herein describes any molecule, e.g. protein or pharmaceutical, with the capability of altering or mimicking the physiological function of a pressure overload associated associated gene. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a-detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to a pressure overload associated gene product, as at least some of the compounds so identified are likely inhibitors. The binding assays usually involve contacting a protein with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots. The protein utilized in such assays can be naturally expressed, cloned or synthesized.

Compounds that are initially identified by any of the foregoing screening methods can be further tested to validate the apparent activity. The basic format of such methods involves administering a lead compound identified during an initial screen to an animal that serves as a model for humans and then determining if an pressure overload associated gene is in fact differentially regulated. The animal models utilized in validation studies generally are mammals. Specific examples of suitable animals include, but are not limited to, primates, mice, and rats.

Active test agents identified by the screening methods described herein can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

Once analogs have been prepared, they can be screened using the methods disclosed herein to identify those analogs that exhibit an increased ability to modulate gene product activity. Such compounds can then be subjected to further analysis to identify those compounds that appear to have the greatest potential as pharmaceutical agents. Alternatively, analogs shown to have activity through the screening methods can serve as lead compounds in the preparation of still further analogs, which can be screened by the methods described herein. The cycle of screening, synthesizing analogs and re-screening can be repeated multiple times.

Compounds identified by the screening methods described above and analogs thereof can serve as the active ingredient in pharmaceutical compositions formulated for the treatment of various disorders, including a propensity for heart failure. The compositions can also include various other agents to enhance delivery and efficacy. The compositions can also include various agents to enhance delivery and stability of the active ingredients.

Thus, for example, the compositions can also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, and intrathecal methods.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

The mammalian heart responds to pressure overload by undergoing left ventricular hypertrophy (LVH) and left atrial enlargement (LAE). The response to pressure overload is mediated in large part by alterations in gene transcription, and previous studies using standard molecular biological, computational, and, recently, microarray techniques have identified a number of genes involved in the pathophysiology of LVH. Many of the differentially expressed genes identified in these earlier studies are involved in cytoskeletal and matrix remodeling, myosin isoform switching (MHCα to MHCβ), TGFβ signaling, and a general reactivation of fetal gene expression patterns. Transcriptional downregulation of components of the fatty acid oxidation pathway in the hypertrophic LV has also been noted, though there has been little previous evidence of alterations in other energy metabolism pathways.

While previous studies have examined transcriptional changes in the LV, almost no attention has been paid to the changes which occur in the other heart chambers in response to pressure overload.

Transverse aortic constriction (TAC) was used to induce LVH and LAE in young adult mice, and then performed genome-wide transcriptional profiling on each of the four heart chambers from TAC and sham operated animals. Transcription of thousands of genes is significantly altered in the hypertrophic LV and enlarged LA, with an unexpectedly dramatic shift in the transcriptional profile of the TAC LA. No significant transcriptional changes are seen in the right atrium or right ventricle. Using Gene Ontology group enrichment analysis, we identified biological process groups with significant changes in group-wide expression, and found major new and unexpected changes in energy metabolism, cell cycle regulation, and signaling pathways in the LA and LV which may profoundly affect our understanding of the molecular basis of the heart's response to pressure overload.

Materials and Methods

Animal surgery, RNA preparation and hybridization. Twenty male FVB mice, age 8 weeks, underwent transverse aortic constriction performed as described by Nakamura et al. (2001) Am J Physiol Heart Circ Physiol. 281:H1104-12; and Rockman et al. (1991) Proc Natl Acad Sci USA. 1991;88:8277-81. Twenty male age matched littermates underwent the identical surgical procedure without placement of the aortic band and served as sham-operated controls.

Hearts were harvested 20 days after operation. Chambers from 15 TAC and 15 sham hearts were divided into three independent pools for RNA isolation (5 mice per pool) to obtain sufficient RNA to perform three biological replicate microarray hybridizations for each chamber. Heart harvest, chamber dissection, RNA preparation, and array hybridizations were performed as previously described in Tabibiazar et al. (2003) Circ Res.

Microarray construction. The Mouse Transcriptome Microarray used in this study was constructed in our laboratory in collaboration with the Stanford Functional Genomics Facility. Briefly, the microarray is composed of 43,200 mouse cDNA probes representing ˜25,000 unique genes and ESTs. It is composed of the National Institutes of Aging 15 k developmental gene set, the Riken 22 k gene set, and approximately 5,000 other unique clones chosen for their biological interest.

Data acquisition, processing, and statistical analysis. Image acquisition, processing, and normalization of the mouse cDNA microarray data was performed as described previously. Microarray experiments were performed using three biological replicates for each tissue and control. Features with values significantly above background in at least two out of three biological replicates were used for two-group statistical comparisons.

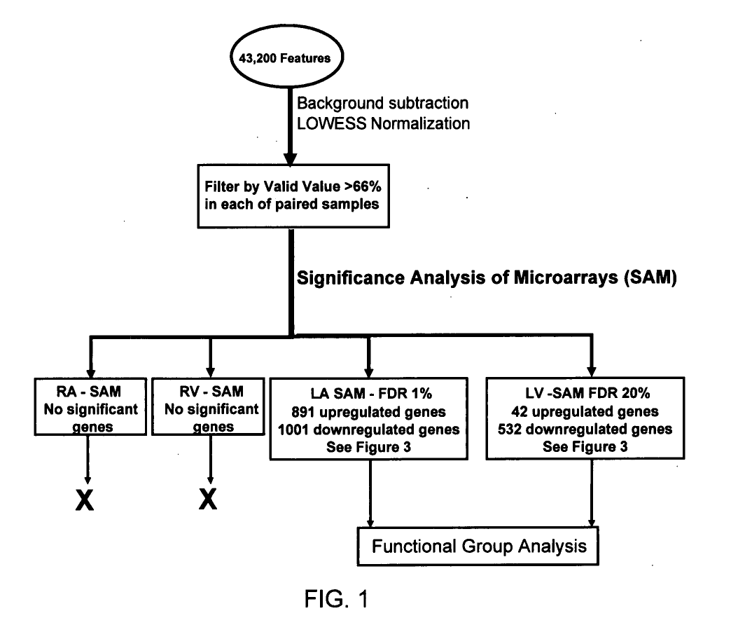

The Significance Analysis of Microarrays (SAM) algorithm was employed to identify genes with statistically different expression levels between TAC and sham for each of the chambers. Hierarchical clustering was performed using a set of variable genes (ANOVA, p<0.005 across all experiments) as described by Tabibiazar et al. (2003), supra. Heat maps were prepared using Heatmap Builder, Version 1. The approach to data analysis is summarized in FIG. 1.

Statistical analysis of over- and under-representation within Gene Ontology categories was performed by applying Fisher's exact test to SAM flagged genes using GoMiner analysis software.

Quantitative real-time reverse transcriptase-polymerase chain reaction. Primers and probes for 9 representative genes were obtained from Applied Biosystems' Assays-on-Demand. Quantitative rtPCR was performed as described by Tabibiazar et al. (2003), supra.

Results

Induction of cardiac hypertrophy. Hearts were harvested 20 days after operative intervention at a point when LV hypertrophy and echocardiographic indices had reached equilibrium (Nakamura et al. (2001) Am J Physiol Heart Circ Physiol. 281:H1104-12). Transverse aortic constriction induced an increase in heart weight of ˜50% (TAC 0.192±0.03 g, sham 0.133±0.007 g, p<0.03), and an increase in heart to body weight ratio of 11% (TAC 5.27+/-0.69, sham 4.72+/-0.32, p<0.03), as expected. On inspection, the left atria and left ventricles of TAC operated animals were visibly greatly enlarged, and the left ventricular wall thickness was increased.

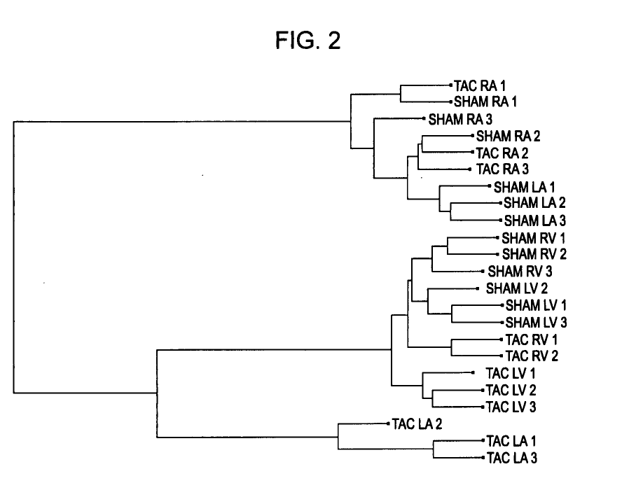

Overview of gene expression patterns—clustering analysis. Twenty-four heart chamber mRNA samples derived from 30 individual animals were labeled and hybridized in triplicate to microarrays containing 42,300 elements, totaling over 1 million gene expression measurements. Hierarchical clustering of the data revealed a large change in the transcriptional profile of the TAC left atria, (FIG. 2) resulting in their clustering more closely with ventricles than with atria. The remainder of the atrial samples clustered as expected, with the sham LA tissues in one subgroup, and TAC and sham RA tissues in another. Left ventricles from TAC mice formed a distinct subcluster within the ventricular group, while the TAC RV and sham RV and LV cluster more closely together, suggesting there is little transcriptional change from the ventricular baseline in these tissues. These clustering results show that the most significant changes in transcription take place in the LA and LV, the two heart chambers most directly affected by increased afterload.

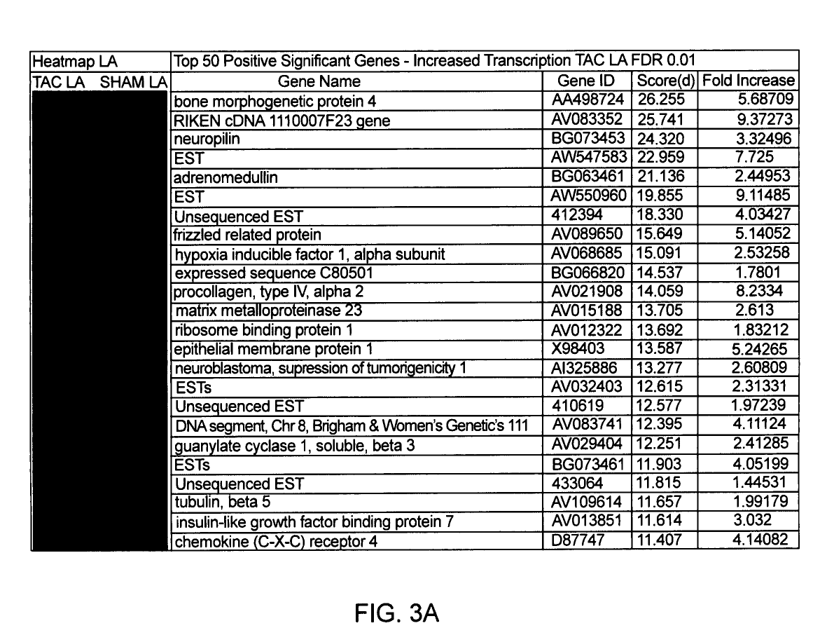

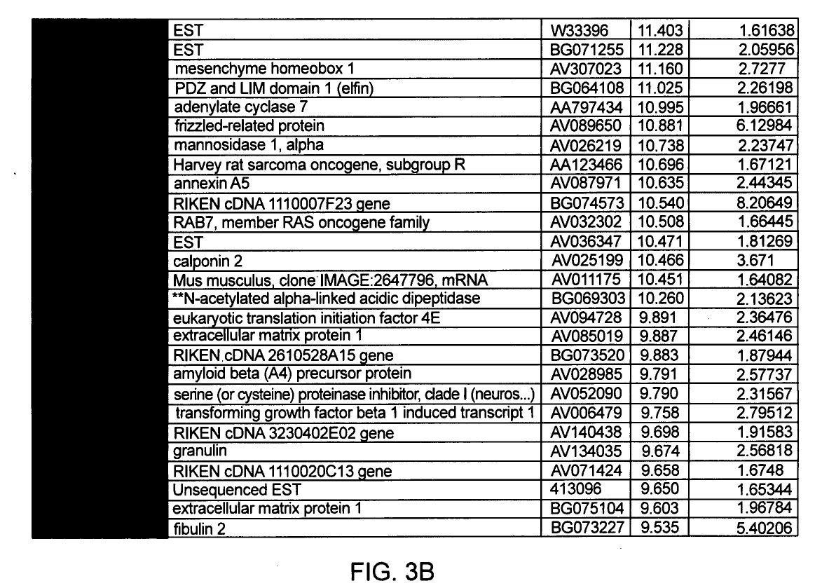

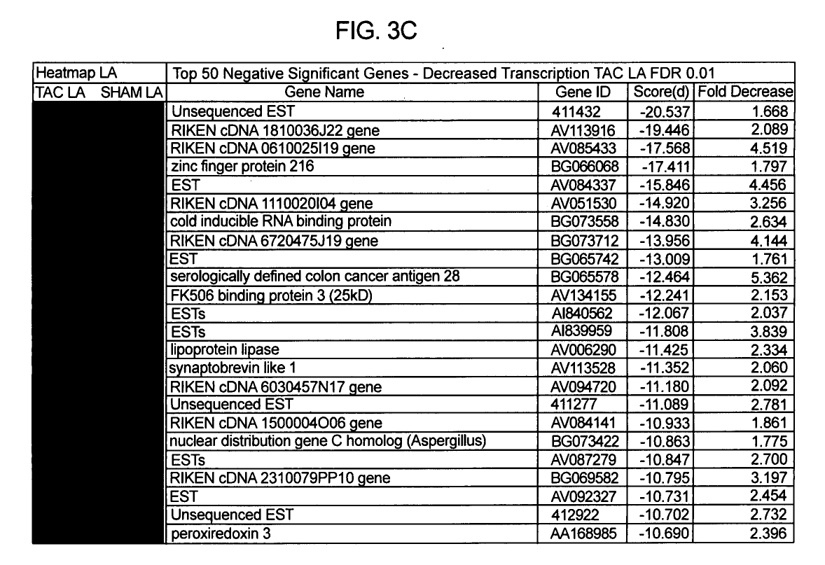

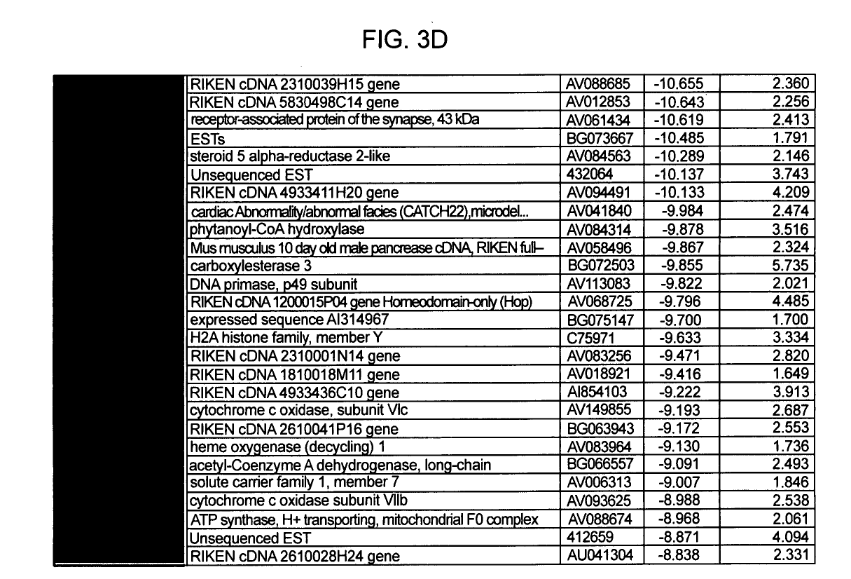

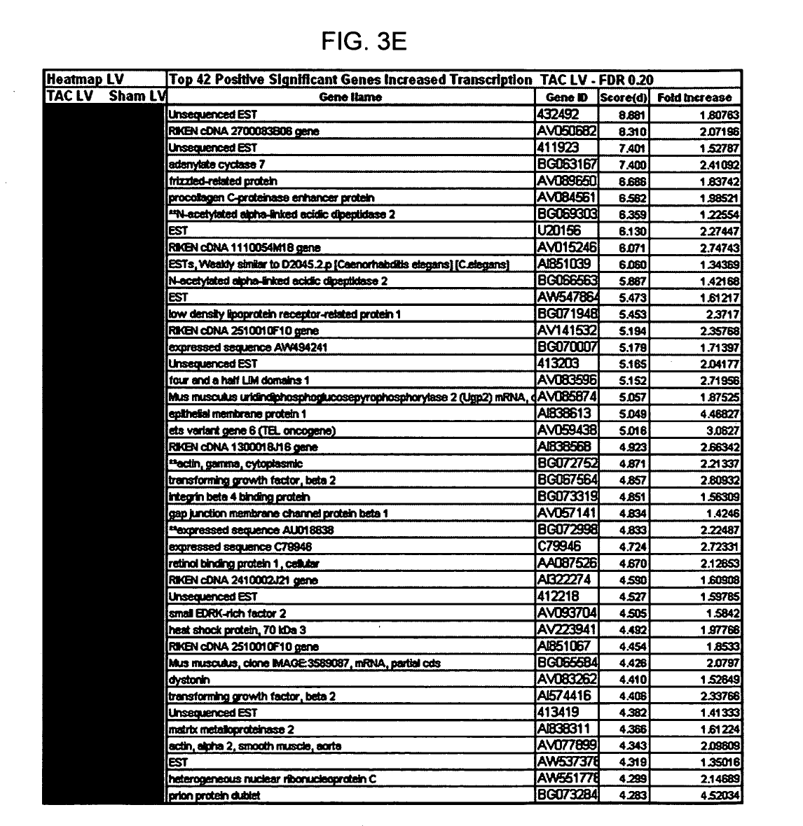

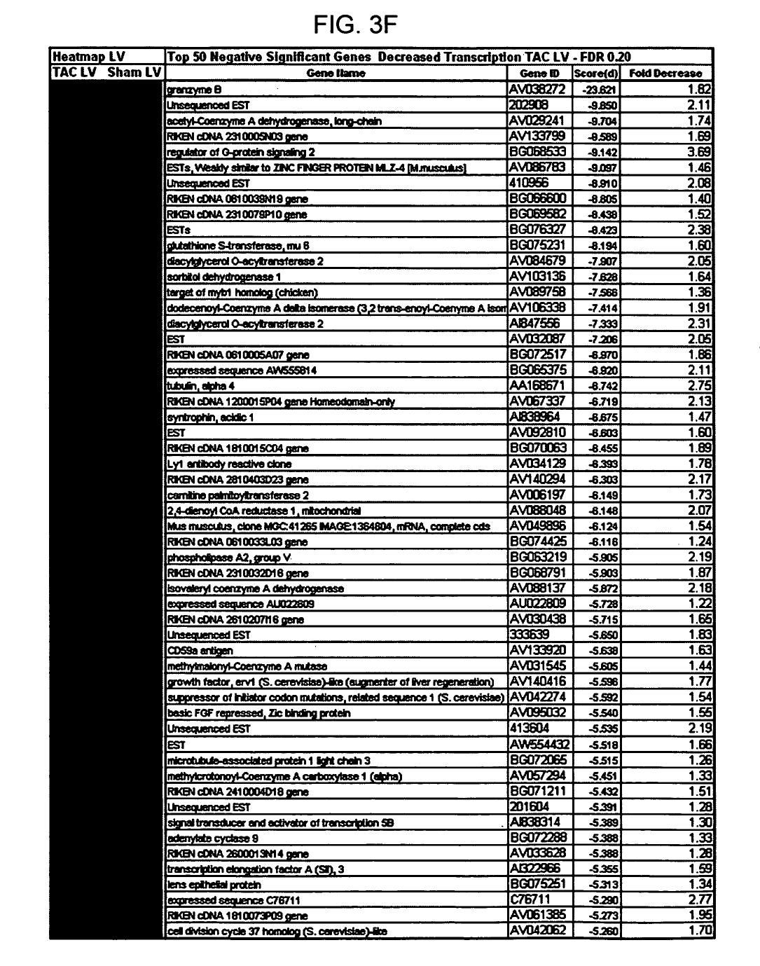

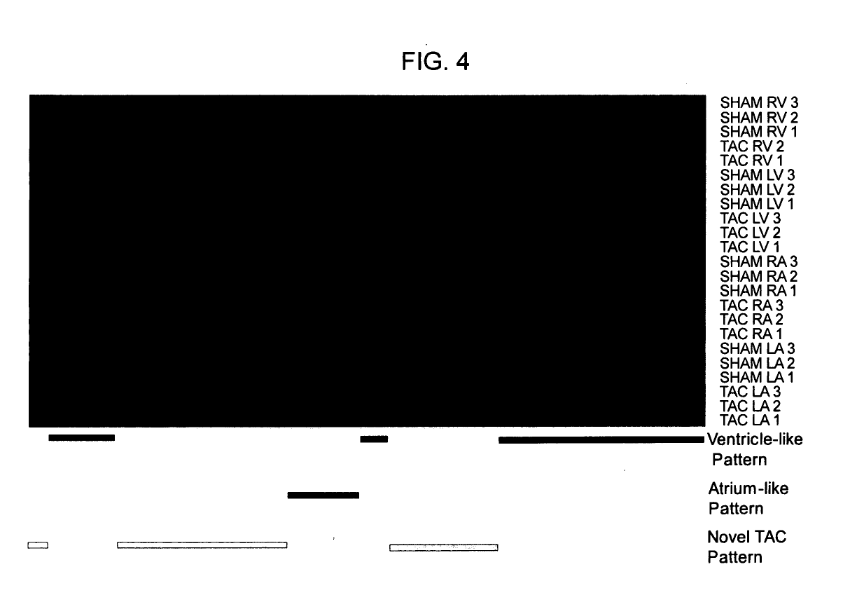

Differential gene expression in the left atria and left ventrcles of TAC mice. Using SAM, we identified 891 upregulated and 1001 downregulated genes in the TAC LA (false detection rate (FDR) <0.01) (FIG. 3a). A heatmap of these variable genes highlights genes whose expression in the TAC LA was similar to the ventricular pattern (FIG. 4). In the LV, SAM identified 42 upregulated and 532 downregulated genes (FDR<0.20)(FIG. 3b). Overall, the differentially regulated genes, and their direction of change in expression, are similar in the LA and LV. SAM analysis of RV and RA data demonstrated that there are no significant differences in gene expression in these tissues. T-tests identified only a small number of genes in the RA and RV with differential expression that trended toward significance.

GO functional group enrichment analysis of differentially regulated genes demonstrates coordinated regulation of biological processes. We applied Fisher's exact test to the 8773 unique GO annotated genes on the array to identify statistically significantly enriched and depleted GO groups in the TAC LA and LV. (FIG. 5). In the TAC LA, among the most significantly upregulated processes were signaling pathway activation, blood vessel development/angiogenesis, cell matrix and adhesion, and cytoskeletal organization. Downregulated processes were dominated in both the TAC LA and LV by energy pathways, including downregulation of genes involved in fatty acid oxidation, the TCA cycle, and oxidative phosphorylation. Because of the small number of upregulated genes in the TAC LV, statistical GO group analysis was not considered to be valid.

Transcriptional regulation of signaling pathways. The physiological stresses of pressure overload must be transduced into molecular signals to actuate compensatory mechanisms in cardiac cells. Deciphering which genes and pathways are involved in this transduction is of central importance, since they are some of the most interesting targets for further investigation and, potentially, drug development. In this study, we have identified many specifically regulated genes from a number of signaling pathways that have not previously been implicated in the pressure overload response.

Signaling through the transforming growth factor-β superfamily pathways is thought to modulate the cardiac response to stress, but the role of many of the downstream molecules has not been well characterized. We found significant increases in the transcription of TGF-β82, BMP2, BMP4, BMP receptor 1A, and endoglin, a component of the TGF-β receptor complex involved in angiogenesis and vessel identity. In addition, transcription of many downstream genes, including TGF-β induced transcript 1, latent transforming growth factor-β binding protein 3, activin receptor-like kinase 1, and SMADs 2, 5, 6, and 7 was significantly increased in the TAC LA, implicating them in the pressure response.

G-protein coupled receptor (GPCR) signaling pathways play a key role in the cardiac response to pressure overload. The most striking finding was the 3.6-fold downregulation of regulator of G-protein signaling 2 (RGS2) in both the LA and LV of banded mice. This gene is critically important in the regulation of blood pressure and vascular smooth muscle relaxation. Expression of the related genes RGS 3, 4, and 5 was significantly upregulated (˜2-fold) in the TAC LA but not LV. Other modifiers of GPCR signaling, the Rho small GTPases, are also specifically regulated in pressure overload. Expression of Rho A2, C, D, and G is highly significantly increased, and Rho GDP dissociation inhibitor alpha, which disrupts cardiac morphogenesis when overexpressed in the heart, is upregulated by 2.5-fold. In total, 7 of 28 annotated Rho signal transduction genes and 22 of 181 small GTPase signal transduction genes are upregulated, suggesting that this signaling pathway is integrally involved in the pressure overload response.

Transcription of several pathways involved in cell-cell signaling and physiological regulation is also dramatically impacted in pressure overload. For example, many components of angiogenic signaling pathways including VEGF A, VEGF C, VEGF-D (fos induced growth factor), neuropilin, TIE 1 tyrosine kinase receptor, angiopoietin 2, endoglin, PDGF receptor beta polypeptide, MCAM, protein O-fucosyltransferase 1, integrin alpha V, endothelial PAS domain protein 1 (HIF 2 alpha), and hypoxia inducible factor 1a are upregulated in the LA, as is chemokine receptor CXCR 4, a transcript directly induced by HIF. Altered hemodynamics in the LA also leads to regulation of a number of vasoactive peptides; transcription of endothelin receptor b was upregulated by 2-fold, while transcription of endothelin itself was downregulated 2-fold. Angiotensin converting enzyme (3,4-fold), angiotensin receptor-like 1 (Apelin receptor)(2,3-fold), adrenomedullin (2.5fold), and myotrophin (3,4-fold) were also upregulated in the LA, suggesting that the left atrium may be especially important in sensing and responding to volume conditions.

Transcriptional Regulation of Downstream Processes

Matrix and cytoskeletal remodeling. In response to the signals documented above, the pressure overloaded heart undergoes substantial tissue and cellular remodeling. Since much of this remodeling is maladaptive, and drugs which interrupt the process promote survival, (Jessup and Brozena (2003) N Engl J Med. 348:2007-18) it is important to understand which specific genes are involved. Many matrix and cell adhesion genes are highly differentially regulated, with expression differences from 5-15 fold. Expression of specific collagens is upregulated (types I, III, IV, V, VI, VIII, XV, XVI, XVIII) or downregulated (types II, IX, XI, XIV, as are specific MMPs (2 and 23 upregulated, 3, 8, 13, and 16 downregulated). One of the most highly regulated ECM genes is osteoblast specific factor 2, which has also been identified in other surveys of pressure overload. In all, more than 40 cell adhesion genes are upregulated in the TAC LA (FIG. 5).

Dynamic cytoskeletal remodeling also occurs in response to pressure overload. Transcription of a large number of actins and other cytoskeletal proteins is highly upregulated in the TAC tissues, including beta cytoplasmic actin, catenin beta, cofilin 1 (non-muscle), alpha actinin 1, coronin, dynein cytoplasmic light chain 1, thymosin beta 4 and 10, tropomodulin 3, calponin 2, destfin, drebrin, epithelial protein lost in neoplasm, vinculin, LIM and SH-3 protein 1, actin related protein complex 2/3 subunits 1B and 3, glia maturation factor beta, moesin, and the atypical, myosins Ic, Va, and X (FIG. 1a). Transcription of several actin related genes including α2 smooth muscle actin, γ-cytoplasmic actin, and four-and-a-half LIM domains 1 is also upregulated in the TAC LV. In the overabundance analyses, 30 of 298 annotated cytoskeletal and structural genes are upregulated in the TAC LA (FIG. 5). This highly specific regulation of a broad range of matrix and cytoskeletal genes demonstrates that the significant remodeling that is taking place is following a precise molecular script.

There are many points at which this maladaptive process be interrupted, such as specific inhibition of matrix metalloproteinases or potentiation of TIMPs, which can provide treatment of new aspects of the disease process.

Precisely regulated expression of cell cycle factors. Another prominent downstream target of signaling in pressure overload is the cell cycle machinery. Over 30 of 328 cell cycle genes are upregulated in the TAC LA; importantly, these genes are a clearly delineated subset of the G1 cell cycle machinery. Transcription of the early G1 cyclins D1 and D2 is elevated 2.4-to 4.7-fold in both the TAC LA and LV while there is no change in the late G1 cyclin E, necessary for entry into S-phase, or cyclin B, necessary for the G2/M phase transition. Inhibition of cyclin D expression or the downstream E2F in primary cardiomyocyte culture has been shown to prevent the development of cardiomyocyte hypertrophy. Thus, it appears that cyclin D/CDK activity without cell cycle progression promotes the hypertrophic response by facilitating increased transcription of prohypertrophic genes. Our finding that this mechanism is active in vivo in the LA and LV indicates that targeted inhibition of D-type cyclin activity provides another therapeutic approach to hypertrophy.

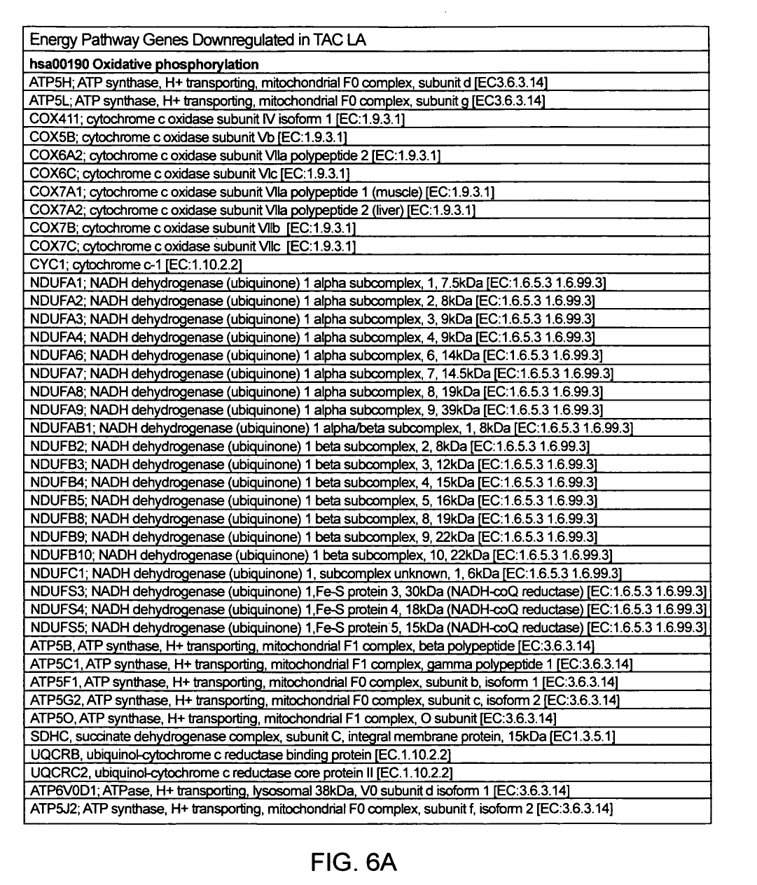

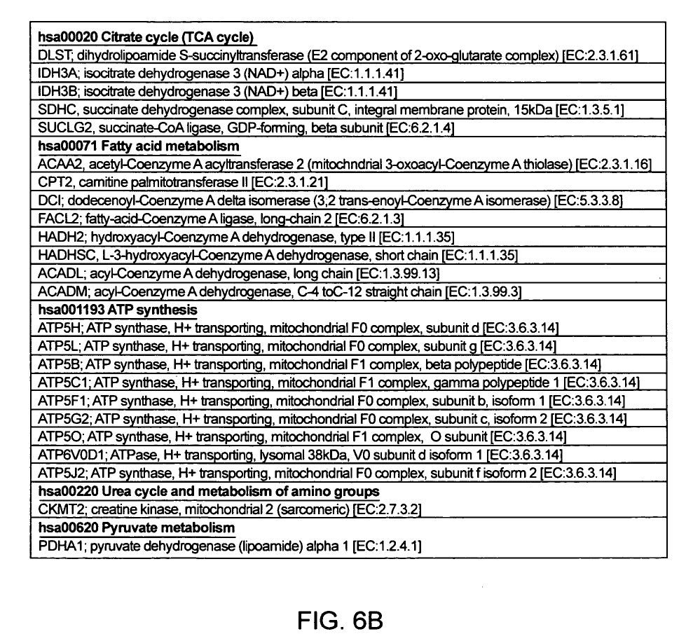

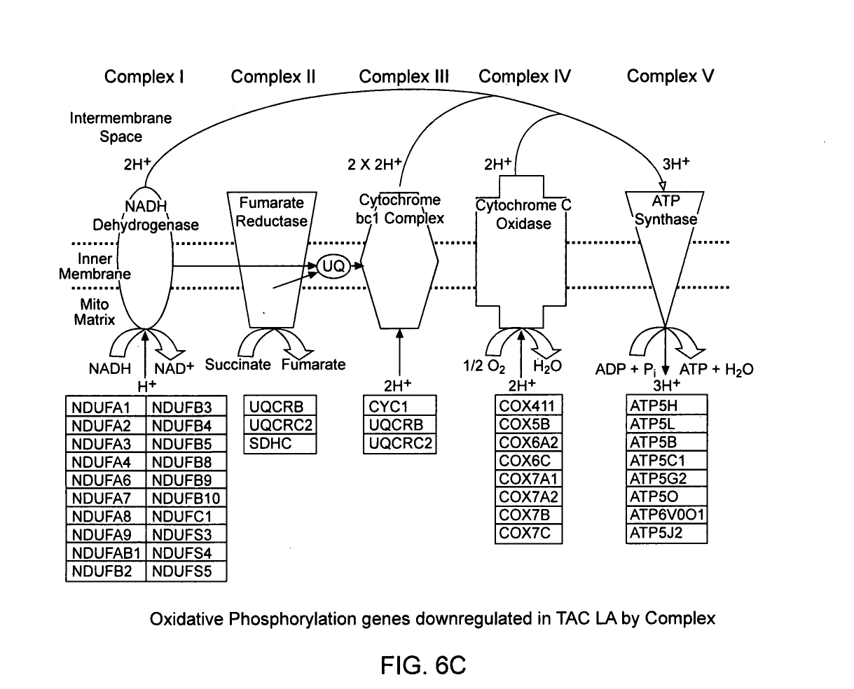

Altered regulation of energy metabolism. One of the most prominent and interesting targets of signaling in the pressure overloaded heart is energy metabolism. In both the LA and LV, there is a major downregulation of mitochondrial oxidative phosphorylation, the TCA cycle, and fatty acid oxidation in the TAC LA and LV. Transcription of over 40 genes associated with complexes (I-V) of the mitochondrial oxidative phosphorylation and respiratory chain machinery is dramatically downregulated, as are 7 TCA cycle genes and a large number of lipid metabolism and fatty acid oxidation pathway genes. (FIGS. 5, 6) These metabolic alterations have profound implications in a signaling feedback mechanism which may perpetuate hypertrophy.

Differential expression of hundreds of uncharacterized ESTs. A major benefit of performing microarray analyses is the ability to recognize new, uncharacterized genes which may be involved in disease processes. We have identified over 200 upregulated and 400 downregulated ESTs which respond to pressure overload. Further analysis of these novel genes can provide unique insights into the biology of the cardiac response to stress.

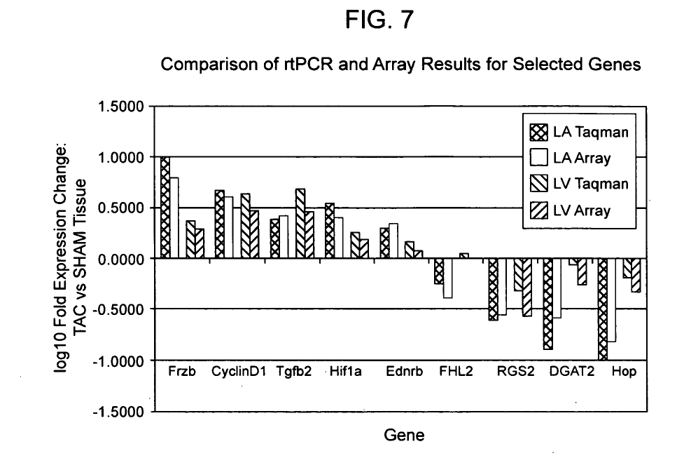

Quantitative realtime polymerase chain reaction confirmation of array results. Quantitative realtime polymerase chain reaction (qRT-PCR) was performed using primers for nine representative genes involved in the major processes discussed to verify that array results represent true expression differences. Each of the genes was shown to be regulated similarly in the qRT-PCR and array measurements, with the qRT-PCR data showing slightly larger measured differences in most cases (FIG. 7).

Heart failure is the leading cause of morbidity in western cultures. Commonly, the disease process begins with the development of LVH and LAE due to an increase in afterload, often as the result of systemic hypertension or aortic valve disease. We have used microarray profiling of the TAC mouse model of pressure overload to obtain a more comprehensive view of the genes and processes involved in the heart's response to increased afterload.

Previous studies of cardiac pressure overload have focused on only one heart chamber, the left ventricle, and have used significantly smaller microarrays. By using more comprehensive microarrays and improved statistical techniques to analyze transcription in the LV, we have been able identify important and previously unrecognized genes, pathways, and processes which mediate changes in the hypertrophic LV.

While the LV takes the brunt of the pressure insult, we know that during pressure overload the left atrium faces physiological challenges due to mitral regurgitation and increased wall stress which result in enlargement and remodeling. Many of the most important clinical complications of hypertrophic cardiomyopathy, valvulvar heart disease, and congestive heart failure are due to atrial enlargement, and include atrial fibrillation and other electrophysiological disturbances, as well as hemodynamic compromise caused by decreased ventricular filling. Knowing which genes and processes are associated with the atrial response may give us important clues about how to intervene in this disease process, but no studies have previously examined the transcriptional changes in the left atrium in this setting. Surprisingly, the transcriptional changes in the enlarged LA are tremendous, and much greater in scope and magnitude than the changes in the LV at this timepoint.

Similarly, no previous studies have examined whether increased pulmonary capillary wedge pressure or systemic neurohumoral changes due to left sided stresses induce transcriptional changes in the right ventricle and atrium. By examining transcription in the RA and RV, we have shown that at this point in the process, which is characterized by substantial left ventricular hypertrophy and left atrial enlargement, transcription in the RA and RV is essentially unchanged.

Our findings provide answers to a number of intriguing questions about the biology of heart failure. We know that physiological stresses such as stretch, shear, and hypoxia must be transduced into cellular signals. The data indicate that a number of different pathways are utilized in specific ways. For example, we see evidence for activation of TGFβ superfamily pathways from the extracellular space (TGFβ2, BMP2 and 4), to cell surface receptors (endoglin, BMP receptor 1a , ACVRL), to downstream transcription factors (SMADs). While the participation of TGFβ itself in the response to pressure overload has been suspected for some time, this is the first demonstration that BMPs and their receptors are involved. Mutations in the BMP pathways may be responsible for inherited cardiomyopathies, and whether targeted myocardial overexpression predisposes the heart to hypertrophy. If so, components of these BMP pathways may be tempting targets for the development of drugs aimed at interrupting the hypertrophic response.

Another unique observation from these investigations is that angiogenic signaling pathways are upregulated in the TAC LA, from extracellular VEGFs A, C and D, to receptors (Tie1, neuropilins), to transcription factors (Hif1α). This is likely the result of increased workload that leads to myocardial hypoxia followed a by robust angiogenic response.

Energy generation in the normal adult myocardium is primarily dependent on oxidative metabolism of long-chain fatty acids through the TCA cycle and mitochondrial oxidative phosphorylation, all of which we find to be dramatically transcriptionally downregulated in both the LA and LV. Though a metabolic substrate switch from fatty acids to glucose in LV hypertrophy is a well known phenomenon, there has been little previous evidence of altered expression of mitochondrial respiratory chain genes with only a few instances of decreased transcription (COX I and IV, adenine nucleotide transporter 1, F1ATPase α and β) or protein levels (ANT1, F1 ATPase α and β cytochrome c oxidase, cytochrome b5) in stressed hearts reported. We find that transcription of more than 40 genes coding for multiple components of all five complexes of the respiratory chain is dramatically downregulated in both the TAC LA and LV (FIG. 5). This concerted metabolic switch from oxygen intensive fatty acid oxidation and oxidative phosphorylation (4.1 mole ATP/1 mole O₂) to glycolysis (6.3 mole ATP/1 mole O₂) probably represents a response to relative hypoxia resulting from increased myocardial work and increased oxygen extraction. This response, however, leads to lower energy production in the form of ATP.

What are the potential effects of this energy deficit on the myocardium? We know that a number of mutations in disparate energy pathway genes such as the mitochondrial fatty acid importer CD36, very long chain acyl-CoA dehydrogenase, adenine nucleotide translocator-1, and mitochondrial tRNA result in inefficient ATP production and lead to hypertrophic cardiomyopathy. Another major class of inherited cardiomyopathies is due to sarcomeric protein mutations, many of which result in inefficient ATP utilization. This has led to the development of a model in which end-systolic ATP depletion prevents effective cytosolic calcium clearance by the SERCA2 pump, which is exquisitely sensitive to ATP levels. Prolonged cytosolic calcium transients then activate calcium sensitive mediators such as calcineurin, calmodulin, and CaM kinase, leading to hypertrophic stimulation.

The dramatic downregulation of oxidative phosphorylation observed herein certainly also leads to decreased ATP production in accordance with this model. The likely proximate cause for downregulation of ox-phos in the pressure overloaded and hypoxic tissues is to prevent the production of immediately toxic reactive oxygen species; unfortunately, this leads to a cycle-of hypertrophy, increased oxygen demand, ATP depletion, and further hypertrophic signaling. (FIG. 8)

The response to cardiac pressure overload requires the coordinated regulation of transcription of thousands of genes in the left atrium and left ventricle. Microarray transcription profiling and rigorous and innovative statistical techniques are used to identify the specific genes and the general biological processes which are modulated in a standard mouse model of LV hypertrophy and LA enlargement. Transcriptional patterns demonstrate significant alterations in energy metabolism, cell cycle regulation, remodeling, and signaling transduction. This study provides important insights into the pathophysiology of LVH and LAE, and identifies numerous new targets diagnosis and therapy.

TABLE I
Significant Genes List - Significantly Altered Expression in Hypertrophic Cardiomyopathy

| | |
|---|---|
| S0 percentile | 0.03 |
| False Significant Number (Median, 90 percentile) | (19.57943, 55.64681) |
| False Discovery Rate (Median, 90 percentile) | (1.03485, 2.94116) |
| Pi0Hat | 0.51525 |

| | | | |
|---|---|---|---|
| Gene Name | Gene ID | Score(d) | Fold Change |

768 Positive Significant Genes_Upregulated

| | | | |
|---|---|---|---|
| **CD8 antigen, beta chain | BG073140 | 4.935952744 | 1.62458 |
| **DNA segment, Chr 1, ERATO Doi 471, expressed | BG067625 | 6.679778765 | 2.17829 |
| **ESTs, Weakly similar to CG1_HUMAN CG1 PROTEIN [H. sapiens] | BG072335 | 5.639596521 | 2.12391 |
| **expressed sequence AI324259 | AA030895 | 5.862670201 | 2.27914 |
| **expressed sequence AW986256 | AW908312 | 4.547379287 | 1.76174 |
| **guanine nucleotide binding protein, alpha 13 | BG073165 | 5.298455537 | 1.78085 |
| **itchy | BG074097 | 5.958778311 | 1.78255 |
| **lymphoid blast crisis-like 1 | BG063325 | 5.481956898 | 1.83237 |
| **N-acetylated alpha-linked acidic dipeptidase 2 | BG069303 | 10.26035569 | 2.13623 |
| **ribophorin 2, related sequence 1 | BG065724 | 4.279942955 | 1.63117 |
| **RIKEN cDNA 1110005E01 gene | BG072956 | 6.320481699 | 2.65102 |
| **RIKEN cDNA 2210419I08 gene | BG072630 | 4.443289031 | 2.74871 |
| **RIKEN cDNA 9130023P14 gene | BG073847 | 4.898954283 | 2.03363 |
| **secreted acidic cysteine rich glycoprotein | BG065013 | 4.305756425 | 5.37944 |
| **selected mouse cDNA on the X | BG075333 | 5.40756834 | 1.96253 |
| a disintegrin and metalloproteinase domain 15 (metargidin) | AI841353 | 6.418564533 | 1.69879 |
| A kinase (PRKA) anchor protein 2 | AV024684 | 9.339968419 | 2.37728 |
| A20 binding inhibitor of NF-kappaB activation-2 | AV051979 | 4.833606233 | 1.36115 |
| actin related protein 2/3 complex, subunit 1B (41 kDa) | AV000246 | 5.339644842 | 3.15358 |
| actin related protein 2/3 complex, subunit 3 (21 kDa) | AV103730 | 4.357179662 | 1.72106 |
| actin, alpha 1, skeletal muscle | AV085882 | 4.680715563 | 2.52776 |
| actin, alpha 2, smooth muscle, aorta | AA815993 | 4.742146264 | 2.50123 |
| adaptor protein complex AP-1, sigma 1 | AV133937 | 5.115943193 | 1.75715 |
| adenylate cyclase 7 | BG063167 | 5.836599536 | 1.97081 |
| ADP-ribosylation factor 2 | AV030860 | 4.970811116 | 1.83182 |
| ADP-ribosylation factor 4 | AV103043 | 4.859284926 | 1.70300 |
| ADP-ribosylation-like factor 6 interacting protein 5 | AV032992 | 5.254319701 | 1.99125 |
| adrenomedullin | BG063461 | 21.13558162 | 2.44953 |
| aldehyde dehydrogenase family 1, subfamily A1 | BG073939 | 5.362174526 | 2.10401 |
| alpha actinin 4 | AA000257 | 8.732257466 | 2.60533 |
| alpha glucosidase 2, alpha neutral subunit | BG074747 | 6.505408498 | 2.20388 |
| amyloid beta (A4) precursor protein | AV028985 | 9.791283359 | 2.57737 |
| amyloid beta (A4) precursor protein-binding, family B, member 2 | BG074998 | 4.702942915 | 1.59024 |
| amyloid beta (A4) precursor-like protein 2 | AV070218 | 5.099119145 | 1.98500 |
| anaphase-promoting complex subunit 5 | AV162432 | 4.760379367 | 2.04115 |
| angiopoietin 2 | BG176309 | 8.307441471 | 1.96272 |
| angiotensin converting enzyme | AV043404 | 6.765684823 | 3.37500 |
| angiotensin receptor-like 1 | AV025146 | 5.137112984 | 2.30047 |
| ankyrin repeat hooked to zinc finger motif | AV233612 | 5.258631025 | 2.31219 |
| annexin A3 | AV218319 | 5.580106736 | 2.46726 |
| annexin A5 | AV087971 | 10.63486669 | 2.44345 |
| annexin A7 | AV083120 | 6.629951533 | 1.67612 |
| antigen identified by monoclonal antibody MRC OX-2 | AV070419 | 9.074059959 | 3.86021 |
| aquaporin 1 | AV025941 | 4.616039959 | 1.60363 |
| ATPase, Cu++ transporting, alpha polypeptide | AV173744 | 4.546259988 | 1.99187 |
| ATPase, H+ transporting, lysosomal 34 kD, V1 subunit D | AU044566 | 8.432452913 | 2.47791 |
| ATPase, H+ transporting, lysosomal 70 kD, V1 subunit A, isoform 1 | AV031502 | 4.300354342 | 1.50397 |
| ATP-binding cassette, sub-family G (WHITE), member 1 | U34920 | 4.75251549 | 2.19022 |
| basigin | BG064525 | 4.767661651 | 1.91891 |
| Bcl-2-related ovarian killer protein | AV086475 | 4.864063728 | 3.01715 |
| beclin 1 (coiled-coil, myosin-like BCL2-interacting protein) | AV104535 | 5.149891952 | 1.43711 |
| benzodiazepine receptor, peripheral | AV087921 | 6.339980832 | 1.76235 |
| beta-2 microglobulin | X01838 | 4.818860152 | 1.51526 |
| biglycan | AV170826 | 4.23050528 | 9.77739 |
| binder of Rho GTPase 4 | AV033754 | 5.435925244 | 1.57561 |
| biregional cell adhesion molecule-related/down-regulated by oncogene | AV140458 | 6.223050315 | 1.90841 |
| block of proliferation 1 | AV055176 | 4.462862768 | 2.03097 |
| bone morphogenetic protein 1 | BG072809 | 5.076200526 | 1.75397 |
| bone morphogenetic protein 2 | AV087036 | 6.312534538 | 1.97717 |
| bone morphogenetic protein 4 | AA498724 | 26.25531622 | 5.68709 |
| bone morphogenetic protein receptor, type 1A | D16250 | 4.802550091 | 1.70860 |
| bridging integrator 3 | AV041000 | 5.021149627 | 1.50525 |
| calcium binding protein P22 | BG069892 | 6.038426191 | 2.12398 |
| calcium binding protein, intestinal | AV089105 | 5.424073635 | 2.85345 |
| calcium channel, voltage-dependent, beta 3 subunit | BG072964 | 6.261620208 | 2.92954 |
| calponin 2 | AV025199 | 10.46579777 | 3.67100 |
| calreticulin | AV105953 | 5.781249515 | 2.81549 |
| calumenin | AV103772 | 8.556760191 | 2.53735 |
| capping protein alpha 1 | AV001105 | 6.759727509 | 2.71943 |
| caspase 6 | AV078409 | 4.712305758 | 1.66628 |
| catalase 1 | AV006202 | 4.789401928 | 1.58530 |
| catenin beta | AA116287 | 4.625727547 | 3.51804 |
| cathepsin D | X52886 | 6.073458864 | 2.36142 |
| CCR4-NOT transcription complex, subunit 8 | AV086227 | 4.323085101 | 1.52705 |
| CD 81 antigen | AV171867 | 5.345211432 | 1.62394 |
| CD24a antigen | BG076069 | 4.489826052 | 2.69550 |
| CD34 antigen | AI893233 | 5.242368789 | 1.99835 |
| Cd63 antigen | AI838302 | 7.516141528 | 1.57199 |
| CD97 antigen | AI325851 | 4.612899255 | 1.49007 |
| cell line NK14 derived transforming oncogene | AV085072 | 7.267896568 | 1.89454 |
| cellular retinoic acid binding protein I | AV109555 | 4.284820548 | 6.21775 |
| chemokine (C-X-C) receptor 4 | D87747 | 11.40652967 | 4.14082 |
| cholinergic receptor, nicotinic, epsilon polypeptide | AV043279 | 6.325648118 | 2.37315 |
| citrate synthase | AV006320 | 4.319928146 | 1.74608 |
| CLIP associating protein 1 | AV043798 | 7.870330961 | 2.45765 |
| coagulation factor II (thrombin) receptor | BG067569 | 6.360824121 | 3.46932 |
| coatomer protein complex, subunit gamma 1 | AV031224 | 4.96823225 | 1.90246 |
| cofilin 1, non-muscle | AV170788 | 4.418502562 | 3.52909 |
| cut-like 1 (Drosophila) | AV138233 | 4.699208238 | 1.90631 |
| cyclin D1 | AA111722 | 8.105067906 | 4.69475 |
| cyclin D2 | AV112821 | 4.804290349 | 2.37763 |
| cyclin-dependent kinase 9 (CDC2-related kinase) | BG073423 | 4.447615705 | 1.37304 |
| cyclin-dependent kinase inhibitor 1A (P21) | AA184368 | 4.925894578 | 2.03325 |
| cystatin C | AV149987 | 4.597603564 | 1.69061 |
| cytochrome P450, 2j6 | AV147446 | 5.623033193 | 1.75987 |
| damage specific DNA binding protein 1 (127 kDa) | BG063543 | 5.159414426 | 1.74271 |
| degenerative spermatocyte homolog (Drosophila) | AV037185 | 5.957462607 | 1.73960 |
| destrin | BG073428 | 4.348798505 | 2.67946 |
| diaphanous homolog 1 (Drosophila) | U96963 | 5.838659607 | 1.91987 |
| diaphorase 1 (NADH) | BG067095 | 4.899045494 | 4.08856 |
| dimethylarginine dimethylaminohydrolase 2 | BG073732 | 5.137410647 | 1.81856 |
| DNA segment, Chr 10, ERATO Doi 398, expressed | BG075070 | 6.143626337 | 1.70405 |
| DNA segment, Chr 17, human D6S45 | AV133629 | 4.211882115 | 1.59857 |
| DNA segment, Chr 5, Bucan 26 expressed | AV069614 | 5.864980176 | 1.33431 |
| DNA segment, Chr 6, Wayne State University 116, expressed | AV025747 | 4.17734088 | 1.78077 |
| DNA segment, Chr 6, Wayne State University 157, expressed | BG063319 | 4.778791053 | 1.37298 |
| DNA segment, Chr 6, Wayne State University 176, expressed | BG074174 | 5.06659014 | 1.61445 |
| DNA segment, Chr 8, Brigham & Women's Genetics 1112 expressed | AV083741 | 12.39491386 | 4.11124 |
| DnaJ (Hsp40) homolog, subfamily B, member 11 | AV103429 | 4.762415879 | 1.59127 |
| dolichyl-di-phosphooligosaccharide-protein glycotransferase | BG074138 | 5.614640775 | 1.93040 |
| downstream of tyrosine kinase 1 | BG075775 | 4.518520078 | 3.49959 |
| drebrin 1 | AI893388 | 6.85211633 | 2.36141 |
| dual adaptor for phosphotyrosine and 3-phosphoinositides 1 | AV026192 | 4.455231001 | 2.98196 |
| E26 avian leukemia oncogene 1, 5′ domain | BG065072 | 4.66168427 | 1.92560 |
| ectonucleotide pyrophosphatase/phosphodiesterase 1 | BG065640 | 4.820720624 | 2.12344 |
| elastin | AV019210 | 4.312030037 | 9.08198 |
| ELAV (embryonic lethal, abnormal vision, Drosophila)-like 1 (Hu antige [symbol] | AV066211 | 6.879063154 | 1.62078 |
| ELK3, member of ETS oncogene family | BE624428 | 5.107654756 | 2.38162 |
| elongation of very long chain fatty acids (FEN1/Elo2, SUR4/Elo3, yeas [symbol] | AV050518 | 4.418412743 | 2.30385 |
| embigin | AV140302 | 4.484360869 | 5.19130 |
| endoglin | AV086531 | 6.471940695 | 2.94673 |
| endothelial cell-selective adhesion molecule | AV104213 | 5.050052051 | 1.60966 |
| endothelial PAS domain protein 1 | AV024401 | 8.285911089 | 3.72721 |
| endothelin receptor type B | AA646322 | 6.145920718 | 2.12895 |
| enhancer of rudimentary homolog (Drosophila) | AV109613 | 6.553746708 | 1.82896 |
| enigma homolog (R. norvegicus) | AV032832 | 4.944256052 | 3.43678 |
| epithelial membrane protein 1 | X98403 | 13.58738841 | 5.24265 |
| epithelial protein lost in neoplasm | AV111531 | 4.531493283 | 1.48848 |
| EST | AW550960 | 19.85526024 | 9.11485 |
| EST | AW547583 | 22.95866337 | 7.72500 |
| EST | AV025040 | 4.957687972 | 6.04194 |
| EST | AW549166 | 4.595440753 | 3.33061 |
| EST | AW554082 | 6.275568831 | 3.30960 |
| EST | S78355 | 4.608423503 | 3.25394 |
| EST | AV109453 | 4.819280814 | 2.92748 |
| EST | AW540995 | 4.418897593 | 2.81516 |
| EST | AW558227 | 5.708451876 | 2.56659 |
| EST | AW546256 | 5.04488313 | 2.47766 |
| EST | AV087039 | 5.166733239 | 2.46773 |
| EST | AW544349 | 6.584770327 | 2.44220 |
| EST | AV039967 | 7.723950024 | 2.43554 |
| EST | AW536421 | 4.60287571 | 2.31306 |
| EST | AV111465 | 8.781751248 | 2.25221 |
| EST | AV088410 | 8.109631088 | 2.25135 |
| EST | AV140901 | 6.233643771 | 2.22461 |
| EST | AV000446 | 7.438718341 | 2.15361 |
| EST | AV171584 | 4.477396404 | 2.15320 |
| EST | BG071255 | 11.22819532 | 2.05956 |
| EST | AW557711 | 4.212906527 | 2.05094 |
| EST | AW537424 | 4.462581095 | 2.00188 |
| EST | AV042683 | 4.743621075 | 1.97510 |
| EST | BG063099 | 4.292752601 | 1.91866 |
| EST | AV083993 | 4.328607976 | 1.88436 |
| EST | AV058573 | 5.408477871 | 1.87775 |
| EST | AV070393 | 6.250654238 | 1.86022 |
| EST | AV111580 | 5.931170364 | 1.85750 |
| EST | AW552177 | 4.265679471 | 1.83036 |
| EST | U20156 | 5.993089117 | 1.81293 |
| EST | AV036347 | 10.47139823 | 1.81269 |
| EST | AV060165 | 4.411955396 | 1.76104 |
| EST | AV094706 | 4.494165965 | 1.66259 |
| EST | AV039638 | 4.503534771 | 1.65226 |
| EST | AW550705 | 4.519430775 | 1.64943 |
| EST | AV034332 | 7.596671753 | 1.62595 |
| EST | W33396 | 11.40348429 | 1.61638 |
| EST | AV011166 | 5.154200811 | 1.52498 |
| EST | BI076464 | 5.448788539 | 1.48872 |
| EST | AI840788 | 5.913183312 | 1.47325 |
| EST | AW548208 | 4.180285767 | 1.45699 |
| EST | AV311582 | 4.533520381 | 1.45416 |
| EST | AV106736 | 4.242664931 | 1.43099 |
| EST | AV015464 | 4.465624384 | 1.38793 |
| EST | AV057158 | 5.371258736 | 1.37442 |
| EST AA087124 | AV087918 | 4.883999133 | 1.86715 |
| EST, Moderately similar to A57474 extracellular matrix protein 1 precu [symbol] | AV087499 | 7.921172215 | 2.38462 |
| ESTs | AV024412 | 4.73782118 | 8.19962 |
| ESTs | BG073461 | 11.90278678 | 4.05199 |
| ESTs | AV033798 | 4.672511285 | 2.61520 |
| ESTs | BG064580 | 5.626668637 | 2.59721 |
| ESTs | BG067879 | 8.66729916 | 2.54050 |
| ESTs | BG076276 | 6.300156668 | 2.48193 |
| ESTs | BG071739 | 8.847636772 | 2.45591 |
| ESTs | AV032403 | 12.61514085 | 2.31331 |
| ESTs | AV078400 | 4.837085255 | 2.27415 |
| ESTs | BG073799 | 8.280866889 | 2.22741 |
| ESTs | BG076404 | 4.634204251 | 2.19874 |
| ESTs | AV014607 | 4.307653699 | 2.06730 |
| ESTs | BG073713 | 6.561139463 | 1.99167 |
| ESTs | BG071422 | 7.424409835 | 1.98279 |
| ESTs | BI076812 | 5.205004314 | 1.85616 |
| ESTs | AV013722 | 5.134325271 | 1.84817 |
| ESTs | AV011768 | 4.642319657 | 1.81806 |
| ESTs | BG068597 | 5.106651008 | 1.80365 |
| ESTs | BG070087 | 4.392989325 | 1.71777 |
| ESTs | AW548360 | 4.447121798 | 1.70141 |
| ESTs | AU040159 | 5.202446948 | 1.64202 |
| ESTs | AV059238 | 4.787621426 | 1.56132 |
| ESTs | BG071674 | 5.550982071 | 1.54806 |
| ESTs, Highly similar to KIAA0356 [H. sapiens] | AU043034 | 5.516554107 | 1.52378 |
| ESTs, Highly similar to tyrosine phosphatase [H. sapiens] | AV085816 | 4.575361973 | 2.50854 |
| ESTs, Moderately similar to AAK1 RAT 5′-AMP-ACTIVATED PROTEIN | AV109623 | 5.911406841 | 2.27280 |
| ESTs, Moderately similar to AF188634 1 F protein [D. melanogaster] | AV083375 | 4.568649007 | 1.95386 |
| ESTs, Moderately similar to KIAA0337 [H. sapiens] | BG074691 | 4.825337515 | 1.56164 |
| ESTs, Moderately similar to S12207 hypothetical protein [M. musculus] | AV024981 | 6.277067603 | 1.92645 |
| ESTs, Moderately similar to T17285 hypothetical protein DKFZp434N0 [symbol] | BG070270 | 4.175752257 | 1.47554 |
| ESTs, Moderately similar to T46312 hypothetical protein DKFZp434J1 [symbol] | BG063981 | 5.614233932 | 1.55378 |
| ESTs, Weakly similar to ATPase, class 1, member a; ATPase 8A2, p t [symbol] | AV021942 | 5.948732902 | 2.18491 |
| ESTs, Weakly similar to DnaJ (Hsp40) homolog, subfamily B, member | AV055460 | 4.218301895 | 1.86141 |
| ESTs, Weakly similar to SELX_MOUSE SELENOPROTEIN X 1 (SELE [symbol] | AA016799 | 4.24930929 | 2.59695 |
| ESTs, Weakly similar to TUBULIN ALPHA-2 CHAIN [M. musculus] | BG069637 | 7.697591957 | 2.61021 |
| ESTs, Weakly similar to TYROSINE-PROTEIN KINASE JAK3 [M. musc [symbol] | BG064647 | 4.824734913 | 1.86704 |
| ESTs, Weakly similar to Y43F4B.7.p [Caenorhabditis elegans] [C. eleg [symbol] | AV016534 | 7.020227711 | 2.36673 |
| ESTs, Weakly similar to ZINC FINGER PROTEIN ZFP-90 [M. musculu [symbol] | AV010028 | 4.601968235 | 2.80189 |
| ETL1 | AV025841 | 5.647091648 | 1.71244 |
| eukaryotic translation initiation factor 4A1 | BG063879 | 4.650336504 | 2.14899 |
| eukaryotic translation initiation factor 4E | AV094728 | 9.89111267 | 2.36476 |
| expressed sequence AA408208 | BG068911 | 4.94103443 | 1.20099 |
| expressed sequence AA408225 | BG064180 | 5.374291641 | 2.50821 |
| expressed sequence AA408783 | AV140475 | 4.763802282 | 2.25681 |
| expressed sequence AA409156 | BG063366 | 8.910555681 | 2.10904 |
| expressed sequence AA414969 | AV024857 | 5.458866268 | 2.29391 |
| expressed sequence AA517451 | BG068828 | 5.023811923 | 1.49100 |
| expressed sequence AA589574 | AV013217 | 4.283226237 | 1.80346 |
| expressed sequence AA960365 | BG063068 | 6.815863912 | 1.66690 |
| expressed sequence AA986889 | AV059924 | 4.234542123 | 2.92099 |
| expressed sequence AI115505 | AV025730 | 7.461892397 | 1.96667 |
| expressed sequence AI316797 | BG072659 | 4.914587425 | 2.36058 |
| expressed sequence AI448102 | AV024096 | 4.73415826 | 1.77000 |
| expressed sequence AI450948 | AW554840 | 4.372618811 | 2.43030 |
| expressed sequence AI451006 | BG064999 | 5.00890408 | 2.04887 |
| expressed sequence AI452336 | AV025047 | 4.324732341 | 1.54836 |
| expressed sequence AI480459 | BG072798 | 4.542252847 | 1.93882 |
| expressed sequence AI481106 | AV025042 | 4.89209432 | 2.42812 |
| expressed sequence AI504145 | AV033704 | 6.252282603 | 1.96397 |
| expressed sequence AI645998 | AV058892 | 6.153140191 | 1.71074 |
| expressed sequence AI790744 | BG075363 | 4.48367478 | 1.83228 |
| expressed sequence AI836219 | AV069461 | 6.473474892 | 1.26115 |
| expressed sequence AI852829 | AV009918 | 7.894529871 | 2.08611 |
| expressed sequence AL024047 | AV103290 | 4.73722655 | 1.67508 |
| expressed sequence AU022349 | BG074257 | 4.17594653 | 1.59209 |
| expressed sequence AU022349 | AV140471 | 4.330667996 | 1.40070 |
| expressed sequence AU022549 | AV037769 | 4.734643112 | 2.21919 |
| expressed sequence AU024550 | AV026341 | 8.658717009 | 1.91059 |
| expressed sequence AV218468 | AV162214 | 4.845939783 | 2.30456 |
| expressed sequence AW146116 | AV087220 | 4.922111816 | 1.82565 |
| expressed sequence AW229038 | BG073479 | 6.074272086 | 5.58416 |
| expressed sequence AW547365 | BG075520 | 4.708552985 | 1.82784 |
| expressed sequence AW553532 | BG074525 | 5.208390615 | 1.92628 |
| expressed sequence C79946 | C79946 | 4.443093726 | 3.00389 |
| expressed sequence C80501 | BG066820 | 14.53712728 | 1.78010 |
| expressed sequence C86807 | BG067580 | 5.813108082 | 1.63424 |
| expressed sequence C87251 | AV010913 | 5.434787975 | 1.62230 |
| expressed sequence R74732 | BG072984 | 5.028448407 | 1.92281 |
| expressed sequence R74732 | AV051721 | 5.134983785 | 1.74936 |
| extracellular matrix protein 1 | AV085019 | 9.887151966 | 2.46146 |
| F-box only protein 25 | AV049438 | 4.694542333 | 1.44710 |
| fibrillin 1 | AA000350 | 4.873526108 | 3.58211 |
| fibroblast growth factor receptor 1 | AW476537 | 5.283837041 | 1.38006 |
| fibronectin 1 | BG072878 | 8.392583287 | 9.10080 |
| fibulin 2 | BG073227 | 9.534808735 | 5.40206 |
| FK506 binding protein 9 | AV059445 | 6.405950764 | 1.82419 |
| flightless I homolog (Drosophila) | AV103121 | 4.923074719 | 2.02616 |
| follistatin-like 3 | BG063294 | 4.93440651 | 2.16520 |
| frizzled-related protein | AV089650 | 10.88058362 | 6.12984 |
| frizzled-related protein | AV089650 | 15.64907314 | 5.14052 |
| FXYD domain-containing ion transport regulator 6 | AV086002 | 5.73258712 | 3.32687 |
| G1 to phase transition 1 | BG066535 | 4.937695403 | 1.78801 |
| GA repeat binding protein, beta 1 | AV041052 | 5.78517292 | 2.14048 |
| gamma-aminobutyric acid (GABA-B) receptor, 1 | AI838468 | 4.537301802 | 1.60145 |
| glia maturation factor, beta | BG066438 | 4.287951378 | 1.91477 |
| glucose regulated protein, 58 kDa | AV073997 | 5.138344434 | 2.95017 |
| glutathione S-transferase, mu 2 | BG076504 | 8.932482655 | 1.89118 |
| glycoprotein galactosyltransferase alpha 1, 3 | BG067028 | 4.369235979 | 2.77433 |
| glycoprotein m6b | AV033394 | 4.391593098 | 2.33415 |
| GPI-anchored membrane protein 1 | AV025862 | 4.623471043 | 2.55428 |
| granule cell differentiation protein - Myotrophin | AV038957 | 6.096480398 | 3.36270 |
| granulin | AV001464 | 5.834497342 | 2.84047 |
| growth arrest and DNA-damage-inducible 45 alpha | AV035081 | 5.53017267 | 1.97603 |
| guanine nucleotide binding protein, alpha inhibiting 2 | BG072092 | 5.46262511 | 2.36297 |
| guanine nucleotide binding protein, beta 1 | BG063447 | 4.468078137 | 2.09860 |
| guanosine diphosphate (GDP) dissociation inhibitor 1 | AV114180 | 5.31572224 | 1.87795 |
| guanosine diphosphate (GDP) dissociation inhibitor 3 | AV141729 | 4.336524933 | 1.59962 |
| guanylate cyclase 1, soluble, beta 3 | AV029404 | 12.25096825 | 2.41285 |
| H2A histone family, member Y | C75971 | 4.826283805 | 1.60582 |
| hairy/enhancer-of-split related with YRPW motif-like | BG063796 | 7.73742705 | 2.82845 |
| Harvey rat sarcoma oncogene, subgroup R | AA123466 | 10.69644502 | 1.67121 |
| heterogeneous nuclear ribonucleoprotein C | AW551778 | 6.086651332 | 4.39239 |
| heterogeneous nuclear ribonucleoprotein K | AV111538 | 5.420454646 | 2.03602 |
| histocompatibility 2, D region locus 1 | X00246 | 4.796300997 | 1.83908 |
| histone deacetylase 1 | AV023621 | 6.399471146 | 1.72915 |
| HLS7-interacting protein kinase | BG064733 | 7.536386645 | 2.10383 |
| homer, neuronal immediate early gene, 3 | AV041850 | 4.333653316 | 1.39983 |
| human immunodeficiency virus type I enhancer binding protein 1 | AI847832 | 5.466729403 | 1.52844 |
| hypothetical protein MGC32441 | AV103742 | 5.697047099 | 1.61848 |
| hypothetical protein MGC7474 | AV025840 | 4.417451505 | 1.54831 |
| hypothetical protein, MGC: 6943 | AV003921 | 4.389090449 | 1.53375 |
| hypoxia inducible factor 1, alpha subunit | AV068685 | 15.09148684 | 2.53258 |
| immunoglobulin kappa chain variable 4 (V4) | AV133863 | 5.61971492 | 1.92740 |
| immunoglobulin superfamily containing leucine-rich repeat | AV084844 | 4.489385861 | 3.04893 |
| inhibitor of DNA binding 2 | BG071421 | 5.645525734 | 2.61535 |
| inositol 1,4,5-triphosphate receptor5 | AI526630 | 5.500524188 | 1.77221 |
| insulin-like growth factor binding protein 5 | AV012617 | 4.210617115 | 1.98780 |
| insulin-like growth factor binding protein 7 | AV013851 | 11.6136427 | 3.03200 |
| integral membrane protein 2B | AV010401 | 4.761131048 | 1.49528 |
| integrin alpha 6 | AV078295 | 4.48185886 | 2.35403 |
| integrin beta 1 (fibronectin receptor beta) | BG074422 | 9.178922865 | 2.31509 |
| integrin beta 5 | BF100414 | 7.042785682 | 4.40899 |
| interferon (alpha and beta) receptor 2 | AV006514 | 6.206846171 | 1.36667 |
| interleukin 17 receptor | AV074586 | 8.887484487 | 2.61352 |
| interleukin 6 signal transducer | BG070387 | 4.905276993 | 3.42328 |
| kit ligand | AV031540 | 4.359720807 | 2.07255 |
| lactate dehydrogenase 1, A chain | AV094945 | 5.610828808 | 2.11934 |
| lamin A | AV057135 | 4.451745488 | 1.91029 |
| laminin, gamma 1 | AA059779 | 5.285143506 | 2.71396 |
| latent transforming growth factor beta binding protein 3 | AV057100 | 7.691066971 | 2.61620 |
| lectin, galactose binding, soluble 8 | AV042964 | 9.342070728 | 1.55241 |
| leptin receptor | AV054666 | 4.245977332 | 1.75594 |
| leukemia-associated gene | AV134166 | 5.334752619 | 2.63905 |
| leukotriene B4 receptor 1 | AV104152 | 4.916931994 | 2.25628 |
| LIM and SH3 protein 1 | AV094974 | 5.827389871 | 2.57319 |
| LIM-domain containing, protein kinase | AV306359 | 5.736847323 | 1.49652 |
| low density lipoprotein receptor-related protein 1 | BG075361 | 8.628798235 | 2.60739 |
| LPS-induced TNF-alpha factor | AV051386 | 4.348912358 | 2.73900 |
| lymphocyte antigen 6 complex, locus A | AV162270 | 4.19767661 | 2.80421 |
| lymphocyte antigen 6 complex, locus E | AV036454 | 4.26829469 | 1.80785 |
| lysyl oxidase-like | AV094998 | 6.168991293 | 3.19925 |
| macrophage migration inhibitory factor | AV099090 | 4.445056769 | 1.46008 |
| MAD homolog 6 (Drosophila) | AA451501 | 5.16784027 | 3.86816 |
| manic fringe homolog (Drosophila) | AV117035 | 7.32646913 | 2.04230 |
| mannosidase 1, alpha | AV026219 | 10.73847163 | 2.23747 |
| matrilin 2 | AV156534 | 4.577038874 | 1.52149 |
| matrix metalloproteinase 2 | M84324 | 7.727668489 | 2.67602 |
| matrix metalloproteinase 23 | BG067807 | 5.424531301 | 1.87576 |
| melanoma cell adhesion molecule | BG075377 | 6.156732011 | 3.94572 |
| membrane-bound transcription factor protease, site 1 | BG072908 | 4.810623416 | 1.93507 |
| mesenchyme homeobox 1 | AV307023 | 11.15999865 | 2.72770 |
| mesothelin | BG074344 | 6.369636518 | 1.59146 |
| metastasis associated 1-like 1 | AV048589 | 4.923977579 | 2.01067 |
| methionine aminopeptidase 2 | AV058243 | 5.461974898 | 2.45077 |
| methyl-CpG binding domain protein 1 | AV029255 | 7.661952699 | 2.16378 |
| microfibrillar associated protein 5 | AV113097 | 6.373883783 | 2.56881 |
| microtubule-associated protein 4 | AV025133 | 6.033347949 | 1.84371 |
| milk fat globule-EGF factor 8 protein | AV094498 | 6.951638445 | 2.53495 |
| milk fat globule-EGF factor 8 protein | AV088358 | 4.283989729 | 1.84505 |
| mitogen activated protein kinase 1 | D10939 | 4.874268557 | 1.57936 |
| mitogen activated protein kinase 3 | BE197033 | 6.398420263 | 1.53070 |
| moesin | BG066632 | 6.70779398 | 1.86464 |
| MORF-related gene X | AV094989 | 5.633228762 | 2.01584 |
| Mus musculus, clone IMAGE: 2647796, mRNA | AV016890 | 6.338916212 | 1.87032 |
| Mus musculus, clone IMAGE: 2647796, mRNA | BG070357 | 6.047190914 | 1.74898 |
| Mus musculus, clone IMAGE: 2647796, mRNA | AV011175 | 10.4511173 | 1.64082 |
| Mus musculus, clone IMAGE: 3597827, mRNA, partial cds | BG071066 | 6.312665533 | 2.57700 |
| Mus musculus, clone IMAGE: 3597827, mRNA, partial cds | AV090253 | 4.407933409 | 1.70877 |
| Mus musculus, clone IMAGE: 4913219, mRNA, partial cds | AI837764 | 4.190999025 | 1.74159 |
| Mus musculus, clone IMAGE: 5066061, mRNA, partial cds | AV025927 | 4.487832407 | 1.99689 |
| Mus musculus, clone IMAGE: 5251262, mRNA, partial cds | AV043496 | 4.810808264 | 2.82307 |
| Mus musculus, clone MGC: 19042 IMAGE: 4188988, mRNA, complete [symbol] | AV073489 | 4.221423402 | 1.62803 |
| Mus musculus, clone MGC: 27672 IMAGE: 4911158, mRNA, complete [symbol] | AV057440 | 4.818077648 | 1.96209 |
| Mus musculus, clone MGC: 36911 IMAGE: 4945500, mRNA, complete [symbol] | BG067972 | 4.567256641 | 1.61513 |
| Mus musculus, clone MGC: 37634 IMAGE: 4990983, mRNA, complete [symbol] | BG063958 | 5.175320148 | 2.15206 |
| Mus musculus, clone MGC: 6357 IMAGE: 3493883, mRNA, complete c [symbol] | BG074005 | 4.309867406 | 2.13653 |
| Mus musculus, clone MGC: 7530 IMAGE: 3492114, mRNA, complete c [symbol] | BG074684 | 4.762369358 | 1.93980 |
| Mus musculus, clone MGC: 7734 IMAGE: 3498403, mRNA, complete c [symbol] | BG073500 | 4.341923916 | 2.21105 |
| Mus musculus, Similar to cytoskeleton-associated protein 4, clone IMA | BG073772 | 5.451341006 | 3.42885 |
| Mus musculus, Similar to gene overexpressed in astrocytoma, clone I [symbol] | BG065693 | 6.47734946 | 2.38394 |
| Mus musculus, Similar to huntingtin interacting protein 1, clone MGC: 2 | BG074730 | 7.373282071 | 1.94462 |
| Mus musculus, Similar to hypothetical protein BC014916, clone MGC: 3 | AU040965 | 5.633541364 | 2.13415 |
| Mus musculus, Similar to hypothetical protein FLJ12806, clone MGC: 6 | AV013963 | 4.728290073 | 2.06908 |
| Mus musculus, Similar to hypothetical protein FLJ20244, clone MGC: 3 | BG064625 | 6.805628105 | 1.67661 |
| Mus musculus, Similar to hypothetical protein FLJ20335, clone MGC: 2 | AV041795 | 4.238385 | 1.55944 |
| Mus musculus, Similar to hypothetical protein MGC2555, clone MGC: 2 | AV089816 | 5.349671441 | 10.06282 |
| Mus musculus, Similar to hypothetical protein MGC3178, clone MGC: 2 | BG065641 | 6.163853471 | 3.84895 |
| Mus musculus, Similar to KIAA1741 protein, clone IMAGE: 5133740, m | BG066559 | 4.277183806 | 1.72731 |
| Mus musculus, Similar to KIAA1741 protein, clone IMAGE: 5133740, m | AV074072 | 5.188066436 | 1.54141 |
| Mus musculus, Similar to pituitary tumor-transforming 1 interacting pro [symbol] | BG066621 | 6.439863345 | 2.07579 |
| Mus musculus, Similar to Protein P3, clone MGC: 38638 IMAGE: 53558 | AV162286 | 4.452893786 | 2.08569 |
| Mus musculus, Similar to Rho GTPase activating protein 1, clone MGC [symbol] | AV009002 | 8.688394673 | 2.37995 |
| Mus musculus, Similar to xylosylprotein beta1, 4-galactosyltransferase, | BG064673 | 4.407048366 | 1.51119 |
| myeloid-associated differentiation marker | BG072632 | 7.785489825 | 1.99411 |
| myosin lc | AW543748 | 4.939976544 | 1.62146 |
| myosin Va | X57377 | 4.179971164 | 2.18490 |
| myosin X | BG065453 | 4.207672452 | 1.44525 |
| myristoylated alanine rich protein kinase C substrate | BG072584 | 8.486813472 | 3.67023 |
| N-acetylated alpha-linked acidic dipeptidase 2 | BG066563 | 5.295722761 | 1.55776 |
| nestin | BG066228 | 4.927494432 | 2.81873 |
| neural proliferation, differentiation and control gene 1 | AV061081 | 7.40303682 | 1.97029 |
| neuroblastoma ras oncogene | BG074219 | 4.631012268 | 2.22671 |
| neuroblastoma, suppression of tumorigenicity 1 | AI325886 | 13.27653071 | 2.60809 |
| neuropilin | AV005825 | 7.420796498 | 4.00358 |
| nidogen 1 | BG063616 | 4.874231512 | 1.63136 |
| Niemann Pick type C2 | BG072810 | 5.871734028 | 2.05727 |
| nischarin | AV024779 | 4.627785218 | 1.86577 |
| nitric oxide synthase 2, inducible, macrophage | M92649 | 6.098182317 | 1.74329 |
| NK2 transcription factor related, locus 5 (Drosophila) | AA530575 | 4.45779765 | 2.08311 |
| N-myc downstream regulated 3 | AV002395 | 6.665100729 | 1.93402 |
| non-POU-domain-containing, octamer binding protein | BG064006 | 4.621685867 | 1.97153 |
| Notch gene homolog 1, (Drosophila) | BF182158 | 4.667460187 | 2.06267 |
| Notch gene homolog 3, (Drosophila) | BF136770 | 4.691872797 | 2.76353 |
| novel nuclear protein 1 | AV030823 | 6.412898231 | 1.45599 |
| nuclear factor of kappa light chain gene enhancer in B-cells 1, p105 | AV011539 | 7.627479907 | 1.72959 |
| nucleobindin | BG067101 | 6.471783836 | 2.20795 |
| O-linked N-acetylglucosamine (GlcNAc) transferase (UDP-N-acetylglu [symbol] | AV026079 | 4.76043905 | 1.79532 |
| origin recognition complex, subunit 2 homolog (S. cerevisiae) | AV032582 | 4.712779251 | 1.52315 |
| osteoblast specific factor 2 (fasciclin I-like) | AV084876 | 6.69600179 | 4.83838 |
| parathyroid hormone receptor | AV145718 | 4.402641605 | 2.07806 |
| parotid secretory protein | BG074915 | 4.353877483 | 1.96222 |
| PDZ and LIM domain 1 (elfin) | AV093772 | 4.260472685 | 2.39615 |
| peptidylprolyl isomerase A | BG065164 | 4.33669464 | 1.87201 |
| peptidylprolyl isomerase C-associated protein | AV059520 | 5.448607935 | 2.69065 |
| peripheral myelin protein, 22 kDa | AV113888 | 7.6004572 | 1.83675 |
| phosphatase and tensin homolog | AI840761 | 4.468842663 | 1.49890 |
| phosphatidylinositol glycan, class Q | AV006019 | 4.310623965 | 1.57576 |
| phosphatidylinositol transfer protein | AV086045 | 9.123016634 | 1.84353 |
| phosphofructokinase, liver, B-type | BG064930 | 5.928386214 | 2.36933 |
| phosphoglycerate mutase 1 | BG064823 | 4.737973813 | 1.87748 |
| phosphoprotein enriched in astrocytes 15 | BG064035 | 4.268230432 | 2.97109 |
| platelet derived growth factor receptor, beta polypeptide | AV112983 | 4.553128201 | 3.77585 |
| platelet-activating factor acetylhydrolase, isoform 1b, alpha1 subunit | AV090194 | 5.288964722 | 1.60210 |
| pleckstrin homology, Sec7 and coiled/coil domains 3 | AV053270 | 5.577033188 | 2.02770 |
| plexin B2 | AW544029 | 4.422870765 | 1.98924 |
| poly A binding protein, cytoplasmic 1 | AV112724 | 4.782371155 | 3.15594 |
| polycystic kidney disease 1 homolog | AV234882 | 5.358502717 | 2.22470 |
| polydomain protein | AI327133 | 7.858540607 | 3.84128 |
| procollagen C-proteinase enhancer protein | AV084561 | 8.995793312 | 3.95693 |
| procollagen C-proteinase enhancer protein | BG074851 | 7.005456302 | 3.30109 |
| procollagen, type IV, alpha 1 | AV009300 | 4.799631432 | 6.90333 |
| procollagen, type IV, alpha 2 | BG074718 | 6.556955707 | 8.64733 |
| procollagen, type XV | AV015595 | 4.255615327 | 1.63778 |
| procollagen-proline, 2-oxoglutarate 4-dioxygenase (proline 4-hydroxyla [symbol] | AW548258 | 4.72698998 | 2.16626 |
| programmed cell death 10 | AV134945 | 4.45010746 | 1.49911 |
| proline arginine-rich end leucine-rich repeat | BG069745 | 5.296255508 | 4.80791 |
| prolyl 4-hydroxylase, beta polypeptide | BG073750 | 4.854848183 | 2.62046 |
| prosaposin | BE307724 | 4.281458018 | 1.86208 |
| prostaglandin-endoperoxide synthase 2 | AV025665 | 6.86188836 | 1.97886 |
| protective protein for beta-galactosidase | AV088011 | 4.408757905 | 1.91973 |
| protein kinase C and casein kinase substrate in neurons 2 | BG074185 | 5.12487867 | 1.71964 |
| protein kinase C, delta | AA276844 | 5.711302904 | 2.37450 |
| protein kinase C, eta | AI787844 | 5.059946731 | 1.93754 |
| protein kinase, cAMP dependent regulatory, type I, alpha | BG075240 | 4.751171639 | 2.91943 |
| protein phosphatase 1, regulatory (inhibitor) subunit 14B | AV087756 | 4.95678378 | 1.55296 |
| protein tyrosine phosphatase, non-receptor type 2 | AA693053 | 9.43234409 | 2.53086 |
| protein tyrosine phosphatase, receptor type, E | BG070083 | 4.670895434 | 1.80602 |
| protein tyrosine phosphatase, receptor type, S | BG074663 | 5.119471562 | 1.71380 |
| proteolipid protein 2 | AI893212 | 4.640045123 | 1.95153 |
| protocadherin 13 | BG073000 | 4.667531323 | 1.89233 |
| protocadherin alpha 1 | AV033049 | 7.668542332 | 1.68190 |
| PTK2 protein tyrosine kinase 2 | BG065137 | 4.202113544 | 1.69356 |
| purine-nucleoside phosphorylase | AU042511 | 4.450485386 | 1.59343 |
| Rab6 interacting protein 1 | AW554976 | 4.29655828 | 1.83268 |
| RAB7, member RAS oncogene family | BG074292 | 8.190446914 | 2.03505 |
| RAD51 homolog (S. cerevisiae) | AV140483 | 4.533421842 | 1.88562 |
| radixin | AV040247 | 4.443038978 | 2.29201 |
| ras homolog 9 (RhoC) | AV140333 | 6.458308062 | 1.82988 |
| ras homolog A2 | AA008793 | 5.650216452 | 1.97274 |
| ras homolog D (RhoD) | AU041357 | 8.369273714 | 1.74085 |
| ras homolog G (RhoG) | AV104284 | 5.754236727 | 1.75346 |
| RAS p21 protein activator 3 | AV090329 | 4.515734577 | 1.43582 |
| Ras suppressor protein 1 | BG064612 | 4.223689279 | 1.66992 |
| regulator of G-protein signaling 19 interacting protein 1 | AV086128 | 5.478596342 | 2.14051 |
| regulator of G-protein signaling 3 | AU040596 | 6.449998123 | 1.32466 |
| regulator of G-protein signaling 4 | AV088379 | 9.080281445 | 2.31400 |
| regulator of G-protein signaling 5 | AV012999 | 6.01259402 | 2.00387 |
| reticulon 4 | AV084219 | 8.227919039 | 2.29694 |
| retinal short-chain dehydrogenase/reductase 1 | BG073341 | 7.334494325 | 1.84661 |
| retinoblastoma binding protein 7 | AW544081 | 4.911862441 | 3.01012 |
| retinoid-inducible serine caroboxypetidase | AV083867 | 7.654642812 | 1.89865 |
| retinol binding protein 1, cellular | AV140184 | 8.194434932 | 2.71765 |
| reversion-inducing-cysteine-rich protein with kazal motifs | AV024396 | 6.204698809 | 2.25801 |
| Rho guanine nucleotide exchange factor (GEF) 3 | AV025023 | 4.811921398 | 2.10195 |
| Rho interacting protein 3 | AV074565 | 9.03990222 | 2.07373 |
| rhotekin | AV170878 | 4.913811275 | 1.99649 |
| ribosomal protein L13a | AV029954 | 7.60434309 | 1.79277 |
| ribosomal protein L35 | AW558719 | 8.648199166 | 1.79930 |
| ribosome binding protein 1 | BG063638 | 4.422386381 | 2.03374 |
| RIKEN cDNA 0610013I17 gene | AW538766 | 7.435056738 | 1.78394 |
| RIKEN cDNA 0610031J06 gene | BG064127 | 5.847627156 | 1.61255 |
| RIKEN cDNA 0610039A15 gene | AV133782 | 4.264872953 | 1.68391 |
| RIKEN cDNA 0610040B21 gene | AV140189 | 4.391354632 | 1.62500 |
| RIKEN cDNA 0610040B21 gene | BG073889 | 4.768851518 | 1.58153 |
| RIKEN cDNA 0610041E09 gene | AV017582 | 5.484190523 | 1.75496 |
| RIKEN cDNA 0710001O03 gene | AV032734 | 5.007378039 | 2.30051 |
| RIKEN cDNA 1100001D10 gene | BG064565 | 5.81906433 | 1.83095 |
| RIKEN cDNA 1110003M08 gene | AV007276 | 4.843292995 | 2.03155 |
| RIKEN cDNA 1110006G06 gene | AV056387 | 4.243506473 | 1.74607 |
| RIKEN cDNA 1110007A10 gene | BG063682 | 5.612559572 | 2.02026 |
| RIKEN cDNA 1110007A14 gene | AV058524 | 9.424689462 | 1.84586 |
| RIKEN cDNA 1110007F23 gene | AV083352 | 25.74086099 | 9.37273 |
| RIKEN cDNA 1110007F23 gene | BG074573 | 10.53962237 | 8.20649 |
| RIKEN cDNA 1110020C13 gene | AV071424 | 9.657620902 | 1.67480 |
| RIKEN cDNA 1110020C13 gene | BG067962 | 4.551573598 | 1.64600 |
| RIKEN cDNA 1110059L23 gene | AV133706 | 5.93034392 | 1.95157 |
| RIKEN cDNA 1110067B02 gene | AV016765 | 4.568660885 | 1.62828 |
| RIKEN cDNA 1110070A02 gene | AV048556 | 4.545063428 | 2.14508 |
| RIKEN cDNA 1190017B18 gene | AV020346 | 4.203168452 | 1.41632 |
| RIKEN cDNA 1200002H13 gene | AV091707 | 4.572821208 | 1.60106 |
| RIKEN cDNA 1200003O06 gene | AV086520 | 4.356732374 | 2.11517 |
| RIKEN cDNA 1200013F24 gene | BG064285 | 4.963857029 | 1.46712 |
| RIKEN cDNA 1200015A22 gene | AV088097 | 5.486213183 | 1.89786 |
| RIKEN cDNA 1200015E15 gene | BG073318 | 5.415048311 | 2.58596 |
| RIKEN cDNA 1200015E15 gene | AV081663 | 6.747503344 | 2.47340 |
| RIKEN cDNA 1200015E15 gene | AV133998 | 7.301986486 | 2.26073 |
| RIKEN cDNA 1200015G06 gene | BG075983 | 5.637931395 | 1.36193 |
| RIKEN cDNA 1300012G16 gene | BG074142 | 4.667358199 | 1.78865 |
| RIKEN cDNA 1300013C10 gene | AV025369 | 6.120894601 | 2.76926 |
| RIKEN cDNA 1300018J16 gene | AI838568 | 4.828416466 | 3.43289 |
| RIKEN cDNA 1500019E20 gene | BG075290 | 4.570907379 | 1.56867 |
| RIKEN cDNA 1600013L13 gene | AV084040 | 4.956392552 | 1.78135 |
| RIKEN cDNA 1600019O04 gene | AV036591 | 6.674797485 | 1.66154 |
| RIKEN cDNA 1600025D17 gene | AV093668 | 5.107066557 | 1.47692 |
| RIKEN cDNA 1810004P07 gene | AV060319 | 5.037144115 | 2.13161 |
| RIKEN cDNA 1810009F10 gene | AV060194 | 5.765496546 | 4.45887 |
| RIKEN cDNA 1810013K23 gene | AV141499 | 4.997925821 | 1.60819 |
| RIKEN cDNA 1810048P08 gene | AV103510 | 5.525945988 | 2.01813 |
| RIKEN cDNA 1810049K24 gene | AV058250 | 4.203974492 | 2.26156 |
| RIKEN cDNA 1810061M12 gene | AV060180 | 5.135166258 | 1.83261 |
| RIKEN cDNA 1810073N04 gene | BG075130 | 4.747837421 | 2.97518 |
| RIKEN cDNA 2010012O16 gene | AV065962 | 4.19570901 | 2.00840 |
| RIKEN cDNA 2010209O12 gene | BG067525 | 4.873273183 | 1.71182 |
| RIKEN cDNA 2210404D11 gene | BG075242 | 4.395009347 | 1.71187 |
| RIKEN cDNA 2210412K09 gene | AV087410 | 4.178520626 | 1.36176 |
| RIKEN cDNA 2210417O06 gene | BG063700 | 4.902542854 | 1.82425 |
| RIKEN cDNA 2300002L21 gene | AV088022 | 5.028858918 | 1.63333 |
| RIKEN cDNA 2310003C10 gene | AV083528 | 4.203309799 | 1.68513 |
| RIKEN cDNA 2310003C10 gene | AV085418 | 4.271031125 | 1.54570 |
| RIKEN cDNA 2310008D10 gene | AV086327 | 7.029577134 | 2.03788 |
| RIKEN cDNA 2310008M10 gene | AV084553 | 6.227559729 | 1.57439 |
| RIKEN cDNA 2310010I22 gene | AV086049 | 6.078943346 | 1.64346 |
| RIKEN cDNA 2310010I22 gene | BG075721 | 4.268018658 | 1.53406 |
| RIKEN cDNA 2310028N02 gene | AV087181 | 5.021775951 | 1.85309 |
| RIKEN cDNA 2310047O13 gene | AV056495 | 4.76990036 | 1.63158 |
| RIKEN cDNA 2310058J06 gene | BG071334 | 6.684567202 | 2.01084 |
| RIKEN cDNA 2410001H17 gene | AV085104 | 4.601565596 | 1.72648 |
| RIKEN cDNA 2410004M09 gene | AV085387 | 4.721414349 | 1.72715 |
| RIKEN cDNA 2410006F12 gene | AV140116 | 5.917743128 | 1.71626 |
| RIKEN cDNA 2410008K03 gene | AV103791 | 4.43380025 | 1.43239 |
| RIKEN cDNA 2410043F08 gene | BG063619 | 8.445139044 | 2.28280 |
| RIKEN cDNA 2410043F08 gene | AV112735 | 9.085975215 | 1.93280 |
| RIKEN cDNA 2500002L14 gene | AV103348 | 5.594034154 | 1.57808 |
| RIKEN cDNA 2500002L14 gene | BG071504 | 4.443376161 | 1.40983 |
| RIKEN cDNA 2510025F08 gene | AV133838 | 4.683564778 | 1.90121 |
| RIKEN cDNA 2510049I19 gene | AV065538 | 4.458739741 | 1.25154 |
| RIKEN cDNA 2600001C03 gene | AV109257 | 6.600191843 | 1.75703 |
| RIKEN cDNA 2600015J22 gene | AI847883 | 4.509126103 | 2.02467 |
| RIKEN cDNA 2610001A11 gene | AV111320 | 4.231568249 | 2.73739 |
| RIKEN cDNA 2610001E17 gene | BG074158 | 5.479986902 | 1.93419 |
| RIKEN cDNA 2610002H11 gene | BG067332 | 4.238835621 | 4.00913 |
| RIKEN cDNA 2610002H11 gene | AV111526 | 4.489291561 | 3.74398 |
| RIKEN cDNA 2610007A16 gene | BG063373 | 5.350241939 | 1.76553 |
| RIKEN cDNA 2610007K22 gene | BG063903 | 4.537443323 | 1.74250 |
| RIKEN cDNA 2610009E16 gene | BG070614 | 4.459754931 | 1.78302 |
| RIKEN cDNA 2610027H02 gene | BG073064 | 4.855351496 | 1.90289 |
| RIKEN cDNA 2610040E16 gene | AV094630 | 4.215693303 | 1.44224 |
| RIKEN cDNA 2610042L04 gene | AV134021 | 7.569249596 | 2.12844 |
| RIKEN cDNA 2610209F03 gene | AV040010 | 4.807860846 | 1.52011 |
| RIKEN cDNA 2610301D06 gene | AV094921 | 4.599529029 | 1.48585 |
| RIKEN cDNA 2610301D06 gene | BG072779 | 4.193665179 | 1.27258 |
| RIKEN cDNA 2610306D21 gene | BG067397 | 4.20266368 | 1.41549 |
| RIKEN cDNA 2610528A15 gene | BG073520 | 9.882601001 | 1.87944 |
| RIKEN cDNA 2700083B06 gene | AV050682 | 5.341326624 | 1.42328 |
| RIKEN cDNA 2810002E22 gene | AV133755 | 5.013779545 | 2.42777 |
| RIKEN cDNA 2810404D13 gene | AV134953 | 5.074203389 | 1.71177 |
| RIKEN cDNA 2810417D08 gene | AV141703 | 4.850126949 | 1.89762 |
| RIKEN cDNA 2810482I07 gene | AV024973 | 5.179744306 | 1.54763 |
| RIKEN cDNA 3110023E09 gene | AV053955 | 4.54999042 | 1.87698 |
| RIKEN cDNA 3110079L04 gene | AV140192 | 8.178677607 | 1.66774 |
| RIKEN cDNA 3230402E02 gene | AV140438 | 9.69822229 | 1.91583 |
| RIKEN cDNA 4432404K01 gene | AV025421 | 6.884470549 | 2.73483 |
| RIKEN cDNA 4833439O17 gene | BG075582 | 4.750554365 | 1.76219 |
| RIKEN cDNA 4921531N22 gene | AV052379 | 6.930339773 | 1.83146 |
| RIKEN cDNA 4921531N22 gene | AV060478 | 5.199122927 | 1.77508 |
| RIKEN cDNA 4930415K17 gene | AV032599 | 5.240194387 | 1.73203 |
| RIKEN cDNA 5031406P05 gene | AV061276 | 6.411675128 | 1.56308 |
| RIKEN cDNA 5033421K01 gene | BG070713 | 4.782136451 | 1.43323 |
| RIKEN cDNA 5133400A03 gene | BG070551 | 4.353282877 | 1.71061 |
| RIKEN cDNA 5430400P17 gene | AA060086 | 6.044644227 | 1.82388 |
| RIKEN cDNA 5730403E06 gene | AV020551 | 4.347632496 | 1.84263 |
| RIKEN cDNA 5730414C17 gene | AV016743 | 4.369181842 | 2.10883 |
| RIKEN cDNA 5730461F13 gene | BG075436 | 6.351981125 | 1.92385 |
| RIKEN cDNA 5730518J08 gene | AV056350 | 4.249685748 | 1.61971 |
| RIKEN cDNA 5730591C18 gene | AV085942 | 4.867612034 | 1.87048 |
| RIKEN cDNA 6030455P07 gene | BG076243 | 5.979146053 | 2.90914 |
| RIKEN cDNA 6330414G21 gene | BG076505 | 4.813930193 | 2.19023 |
| RIKEN cDNA 6720474K14 gene | AV085966 | 4.822592598 | 2.07363 |
| RIKEN cDNA 9130005N14 gene | AV060665 | 4.252358329 | 2.54257 |
| RIKEN cDNA B430104H02 gene | AV000213 | 9.138694463 | 2.32483 |
| RIKEN cDNA C330007P06 gene | AV029419 | 5.722192826 | 1.77950 |
| ring finger protein 13 | AV072479 | 5.989110349 | 1.56109 |
| RNA polymerase II 1 | AV018343 | 4.489707981 | 1.82930 |
| roundabout homolog 1 (Drosophila) | AV128328 | 5.524511639 | 1.85130 |
| roundabout homolog 4 (Drosophilia) | BE377723 | 4.981917421 | 2.15467 |
| RuvB-Iike protein 2 | AV109340 | 4.2446986 | 1.65863 |
| S-adenosylmethionine decarboxylase 1 | AV121939 | 5.707603849 | 1.64498 |
| sarcoglycan, epsilon | BG072850 | 4.370750746 | 1.50031 |
| scavenger receptor class B1 | U37799 | 4.50358952 | 2.46176 |
| secreted acidic cysteine rich glycoprotein | AW988741 | 5.549292892 | 6.14126 |
| secreted frizzled-related sequence protein 2 | AV021712 | 4.238424177 | 3.26213 |
| sema domain, immunoglobulin domain (Ig), short basic domain, secret | BG074382 | 5.028318471 | 2.13790 |
| septin 2 | AV116832 | 7.212302484 | 2.33584 |
| serine (or cysteine) proteinase inhibitor, clade F (alpha-2 antiplasmin, [symbol] | BG074697 | 8.856683533 | 3.35898 |
| serine (or cysteine) proteinase inhibitor, clade H (heat shock protein 47 [symbol] | AV104522 | 4.258740241 | 5.50558 |
| serine (or cysteine) proteinase inhibitor, clade I (neuroserpin), member | AV052090 | 9.790229028 | 2.31567 |
| serine palmitoyltransferase, long chain base subunit 1 | AV062462 | 9.24035025 | 1.73956 |
| serine protease inhibitor 6 | AV035785 | 4.308010944 | 1.41468 |
| serum/glucocorticoid regulated kinase | AI315589 | 4.359268623 | 2.04271 |
| serum-inducible kinase | AV056942 | 8.688448107 | 3.20116 |
| SH3 domain protein D19 | BG076318 | 4.83286573 | 1.72859 |
| shroom | BG072834 | 4.460051279 | 2.66437 |
| sialyltransferase 1 (beta-galactoside alpha-2,6-sialyltransferase) | D16106 | 6.392086396 | 1.92378 |
| sialyltransferase 4C (beta-galactosidase alpha-2,3-sialytransferase) | AI385650 | 6.610358353 | 1.97374 |
| signal transducer and activator of transcription 6 | L47650 | 6.315908147 | 1.91050 |
| signal transducing adaptor molecule (SH3 domain and ITAM motif) 2 | AV046859 | 4.327158168 | 1.76305 |
| signal-induced proliferation associated gene 1 | AV088479 | 4.550408961 | 2.31046 |
| small GTPase, homolog (S. cerevisiae) | BG067356 | 4.586503857 | 1.50828 |
| solute carrier family 29 (nucleoside transporters), member 1 | BG075739 | 4.337648607 | 1.39981 |
| sorting nexin 4 | AV055722 | 4.473535794 | 1.46762 |
| sprouty homolog 4 (Drosophila) | AA499432 | 6.438240138 | 2.13976 |
| SRY-box containing gene 18 | AA261240 | 5.111004932 | 1.78753 |
| stanniocalcin 2 | AV094416 | 4.405714011 | 1.46040 |
| stromal cell derived factor 1 | BG073593 | 4.24723061 | 2.11053 |
| stromal cell derived factor 4 | AV048780 | 4.802035607 | 1.43164 |
| superoxide dismutase 3, extracellular | U38261 | 7.250231972 | 3.29160 |
| suppressor of white apricot homolog 2-pending | AV162195 | 4.994355697 | 1.70716 |
| surfeit gene 4 | AV074505 | 4.815569801 | 1.79779 |
| survival motor neuron | AV133987 | 6.539797582 | 1.39888 |
| SWI/SNF related, matrix associated, actin dependent regulator of chro [symbol] | AV298569 | 4.355370118 | 2.60646 |
| syndecan 3 | BG064265 | 6.613530318 | 2.88308 |
| synovial sarcoma translocation, Chromosome 18 | AV033310 | 5.408808458 | 1.80124 |
| syntaxin binding protein 2 | BG075753 | 5.004233958 | 1.65309 |
| TAR (HIV) RNA binding protein 2 | AV040847 | 6.423086255 | 2.01946 |
| thymic stromal-derived lymphopoietin, receptor | AV070805 | 8.547082806 | 2.02117 |
| torsin family 3, member A | AV057827 | 7.477887867 | 2.27552 |
| transcription factor 4 | AV000162 | 8.345957891 | 2.23130 |
| transcription factor Dp 1 | AV053081 | 4.329499465 | 1.34063 |
| transcription factor E2a | AA030885 | 6.525307406 | 1.75147 |
| transcription factor UBF | AV095317 | 4.895225679 | 1.62658 |
| transforming growth factor beta 1 induced transcript 1 | AV006479 | 9.758134935 | 2.79512 |
| transforming growth factor, beta 2 | AV135894 | 5.173585005 | 2.73350 |
| transient receptor protein 2 | AV002597 | 5.333447366 | 2.68369 |
| transmembrane domain protein regulated in adipocytes 40 kDa | AV083947 | 5.088665302 | 1.28986 |
| transmembrane protein with EGF-like and two follistatin-like domains 1 [symbol] | AA023493 | 5.206812136 | 1.93718 |
| tropomodulin 3 | AV026409 | 5.07481845 | 1.77695 |
| tubby like protein 4 | AW552694 | 4.530630076 | 1.78186 |
| tubby-like protein 3 | AV139648 | 5.616340312 | 1.85776 |
| tubulin, alpha 1 | AV093632 | 6.193575886 | 3.07888 |
| tubulin, alpha 4 | AA408725 | 7.155536699 | 2.13397 |
| tubulin, beta 5 | AV109614 | 11.6573826 | 1.99179 |
| tumor necrosis factor | X02611 | 6.428930694 | 1.53428 |
| tumor necrosis factor receptor superfamily, member 1a | L26349 | 6.392431179 | 2.39873 |
| tumor necrosis factor, alpha-induced protein 1 (endothelial) | AV024570 | 4.370295461 | 1.75306 |
| tumor-associated calcium signal transducer 1 | AV089835 | 6.791092517 | 3.32950 |
| tyrosine 3-monooxygenase/tryptophan 5-monooxygenase activation pr [symbol] | AV104266 | 6.100287629 | 1.55178 |
| tyrosine 3-monooxygenase/tryptophan 5-monooxygenase activation pr [symbol] | U57311 | 6.573928853 | 1.87425 |
| tyrosine 3-monooxygenase/tryptophan 5-monooxygenase activation pr [symbol] | AV130451 | 8.350838932 | 2.79631 |
| tyrosine kinase receptor 1 | AA838996 | 6.050255188 | 3.70273 |
| U1 small nuclear ribonucleoprotein 70 kDa polypeptide A | AV035403 | 5.218365194 | 1.76839 |
| ubiquitin carboxy-terminal hydrolase L1 | BG074009 | 4.758072234 | 2.59745 |
| UDP-GlcNAc:betaGal beta-1,3-N-acetylglucosaminyltransferase 1 | BG062994 | 4.784175093 | 1.63427 |
| UDP-glucuronate decarboxylase 1 | BG073697 | 4.651857039 | 1.53280 |
| UDP-N-acetyl-alpha-D-galactosamine:polypeptide N-acetylgalactosam | AI893181 | 4.61960655 | 1.98472 |
| UDP-N-acetyl-alpha-D-galactosamine:polypeptide N-acetylgalactosam | BG071100 | 5.251330578 | 2.12686 |
| Unsequenced EST | 413107 | 6.273291655 | 7.53126 |
| Unsequenced EST | 413273 | 4.31807147 | 5.78325 |
| Unsequenced EST | 412394 | 18.32998763 | 4.03427 |
| Unsequenced EST | 411467 | 4.357834225 | 3.38896 |
| Unsequenced EST | 411755 | 4.951849941 | 3.34666 |
| Unsequenced EST | 412745 | 4.568501936 | 3.27897 |
| Unsequenced EST | 432151 | 4.774738602 | 2.87892 |
| Unsequenced EST | 432603 | 4.333142623 | 2.85312 |
| Unsequenced EST | 431006 | 6.562712284 | 2.77119 |
| Unsequenced EST | 411350 | 9.505971157 | 2.72549 |
| Unsequenced EST | 411609 | 4.71354952 | 2.66098 |
| Unsequenced EST | 412246 | 5.633966439 | 2.61787 |
| Unsequenced EST | 411505 | 5.901191293 | 2.55842 |
| Unsequenced EST | 432010 | 5.557544512 | 2.54505 |
| Unsequenced EST | 410993 | 4.939733861 | 2.50496 |
| Unsequenced EST | 412701 | 4.209083529 | 2.47011 |
| Unsequenced EST | 411885 | 6.186881729 | 2.40448 |
| Unsequenced EST | 412021 | 4.902811974 | 2.39953 |
| Unsequenced EST | 410761 | 4.924640447 | 2.39667 |
| Unsequenced EST | 431651 | 5.237876041 | 2.38955 |
| Unsequenced EST | 199450 | 5.780625675 | 2.37856 |
| Unsequenced EST | 412588 | 4.795004918 | 2.37853 |
| Unsequenced EST | 411923 | 8.396940653 | 2.33231 |
| Unsequenced EST | 410840 | 4.457849585 | 2.31171 |
| Unsequenced EST | 430732 | 5.597887132 | 2.30696 |
| Unsequenced EST | 412675 | 4.815014954 | 2.22233 |
| Unsequenced EST | 410968 | 5.153844667 | 2.19677 |
| Unsequenced EST | 412594 | 5.824024683 | 2.19605 |
| Unsequenced EST | 410746 | 5.973693751 | 2.18081 |
| Unsequenced EST | 431888 | 8.608487166 | 2.15587 |
| Unsequenced EST | 431920 | 5.682201344 | 2.12745 |
| Unsequenced EST | 410743 | 4.439738415 | 2.12029 |
| Unsequenced EST | 197104 | 8.383105866 | 2.09296 |
| Unsequenced EST | 430919 | 4.794214749 | 2.08514 |
| Unsequenced EST | 431706 | 6.304117743 | 2.08389 |
| Unsequenced EST | 410654 | 8.351953022 | 2.05228 |
| Unsequenced EST | 206956 | 5.237784101 | 2.04248 |
| Unsequenced EST | 193306 | 4.945515669 | 2.02954 |
| Unsequenced EST | 431072 | 5.684602565 | 2.00932 |
| Unsequenced EST | 413009 | 6.614854617 | 1.99915 |
| Unsequenced EST | 411412 | 4.868030026 | 1.99180 |
| Unsequenced EST | 431050 | 6.699411715 | 1.98252 |
| Unsequenced EST | 410619 | 12.57706405 | 1.97239 |
| Unsequenced EST | 411013 | 4.960471191 | 1.96703 |
| Unsequenced EST | 411635 | 6.118763105 | 1.95047 |
| Unsequenced EST | 431767 | 5.521076531 | 1.94831 |
| Unsequenced EST | 411464 | 5.02732744 | 1.94358 |
| Unsequenced EST | 410545 | 6.37147916 | 1.89709 |
| Unsequenced EST | 411329 | 5.294206879 | 1.88701 |
| Unsequenced EST | 411969 | 4.92425749 | 1.86985 |
| Unsequenced EST | 411285 | 4.3570354 | 1.86488 |
| Unsequenced EST | 432326 | 7.966893738 | 1.84998 |
| Unsequenced EST | 412447 | 4.260473196 | 1.83558 |
| Unsequenced EST | 431082 | 4.937632166 | 1.82592 |
| Unsequenced EST | 431540 | 6.428336919 | 1.82275 |
| Unsequenced EST | 196552 | 5.793122078 | 1.81776 |
| Unsequenced EST | 410789 | 4.550275542 | 1.81343 |
| Unsequenced EST | 412803 | 4.176585206 | 1.80861 |
| Unsequenced EST | 411561 | 4.605900103 | 1.80665 |
| Unsequenced EST | 413042 | 4.676182648 | 1.78983 |
| Unsequenced EST | 412220 | 5.167673303 | 1.78385 |
| Unsequenced EST | 207914 | 5.173303361 | 1.76367 |
| Unsequenced EST | 412958 | 4.871233065 | 1.72164 |
| Unsequenced EST | 410773 | 5.107733423 | 1.71129 |
| Unsequenced EST | 432024 | 4.432735142 | 1.70615 |
| Unsequenced EST | 412011 | 4.742393759 | 1.69693 |
| Unsequenced EST | 411472 | 4.490487626 | 1.69603 |
| Unsequenced EST | 411765 | 4.556559515 | 1.69434 |
| Unsequenced EST | 412337 | 4.770108721 | 1.69362 |
| Unsequenced EST | 410698 | 4.340616492 | 1.69179 |
| Unsequenced EST | 413591 | 4.59016315 | 1.68542 |
| Unsequenced EST | 412313 | 4.490810017 | 1.67931 |
| Unsequenced EST | 410920 | 6.621227261 | 1.66619 |
| Unsequenced EST | 412612 | 6.354130371 | 1.65767 |
| Unsequenced EST | 413096 | 9.649532409 | 1.65344 |
| Unsequenced EST | 411309 | 5.855658163 | 1.65342 |
| Unsequenced EST | 431982 | 4.428555085 | 1.63322 |
| Unsequenced EST | 411222 | 4.524397103 | 1.63149 |
| Unsequenced EST | 412210 | 4.357035656 | 1.60479 |
| Unsequenced EST | 413582 | 6.172475352 | 1.59892 |
| Unsequenced EST | 413181 | 5.247839338 | 1.59329 |
| Unsequenced EST | 432273 | 5.284928181 | 1.57465 |
| Unsequenced EST | 411229 | 4.606022357 | 1.55993 |
| Unsequenced EST | 432889 | 6.86044512 | 1.54569 |
| Unsequenced EST | 411240 | 4.931389088 | 1.54312 |
| Unsequenced EST | 411256 | 4.370621835 | 1.53806 |
| Unsequenced EST | 431197 | 5.553558202 | 1.51658 |
| Unsequenced EST | 411384 | 4.226502978 | 1.51562 |
| Unsequenced EST | 433064 | 11.81517212 | 1.44531 |
| Unsequenced EST | 411576 | 4.557199497 | 1.41029 |
| Unsequenced EST | 430683 | 4.395744711 | 1.40057 |
| Unsequenced EST | 207209 | 5.462293397 | 1.39444 |
| Unsequenced EST | 413286 | 6.146895859 | 1.38486 |
| Unsequenced EST | 411904 | 4.653902177 | 1.37670 |
| Unsequenced EST | 333870 | 4.973207701 | 1.33528 |
| Unsequenced EST | 413172 | 4.587654857 | 1.20891 |
| uridine phosphorylase | D44464 | 4.407420784 | 3.33647 |
| valosin containing protein | BG074307 | 4.582529317 | 1.50710 |
| vanilloid receptor-like protein 1 | BG064510 | 5.54598292 | 1.95257 |
| vascular endothelial growth factor A | AW913188 | 8.832564999 | 2.38847 |
| vascular endothelial growth factor C | BE376968 | 6.23701522 | 1.95868 |
| vasodilator-stimulated phosphoprotein | AW538871 | 5.171791268 | 1.99901 |
| vinculin | AI385712 | 4.203457851 | 1.61965 |
| v-rel reticuloendotheliosis viral oncogene homolog A, (avian) | AV095204 | 4.443651896 | 1.71953 |
| WD repeat domain 1 | BG064839 | 5.053585228 | 2.13577 |
| zinc finger protein 103 | AV224747 | 5.236448071 | 1.82055 |
| zinc finger protein 106 | AV071915 | 5.082827154 | 2.05709 |
| zinc finger protein 36 | AV103195 | 4.444107655 | 2.24632 |
| zyxin | AV166088 | 6.273023884 | 1.64875 |

896 Negative Significant Genes - Repressed in Hypertrophic Cardiomyopathy

| | | | |
|---|---|---|---|
| **DNA segment, Chr 13, ERATO Doi 332, expressed | BG066890 | −5.396062055 | 0.45499 |
| **DNA segment, Chr 2, ERATO Doi 542, expressed | BG073740 | −6.995498483 | 0.57935 |
| **DNA segment, Chr 2, Wayne State University 85, expressed | BG062980 | −4.136751331 | 0.61115 |
| **DNA segment, Chr 8, Brigham & Women's Genetics 1112 expressed [symbol] | BG064137 | −4.174714082 | 0.64681 |
| **ESTs | BG074866 | −5.813263409 | 0.54492 |
| **guanine nucleotide binding protein, alpha 13 | BG068913 | −5.745250343 | 0.64597 |
| **methionine aminopeptidase 2 | BG074258 | −5.880170454 | 0.70541 |
| **Mus musculus, clone IMAGE: 5361283, mRNA, partial cds | AA072842 | −4.13161274 | 0.58861 |
| **proteasome (prosome, macropain) 26S subunit, ATPase 3 | AA163174 | −5.040496567 | 0.46827 |
| **RIKEN cDNA 2310075M17 gene | AI840674 | −5.823426143 | 0.68802 |
| **RIKEN cDNA 3110052N05 gene | BG072585 | −4.203653088 | 0.68898 |
| **RIKEN cDNA 3930401B19 gene | BG076041 | −4.221966232 | 0.69199 |
| **RIKEN cDNA 6720463E02 gene | BG067712 | −5.527362247 | 0.42232 |
| **RIKEN cDNA 6720475J19 gene | BG071484 | −7.674685475 | 0.26086 |
| **RNA polymerase II 4 (14 kDa subunit) | BG073536 | −4.407989935 | 0.64966 |
| **small nuclear ribonucleoprotein N | AI841348 | −4.56247846 | 0.50950 |
| **succinate-Coenzyme A ligase, GDP-forming, beta subunit | BG075548 | −4.444081173 | 0.49038 |
| **suppressor of initiator codon mutations, related sequence 1 (S. cere [symbol] | BG064153 | −5.434802411 | 0.46790 |
| **ubiquinol-cytochrome c reductase core protein 1 | AI841290 | −4.554338409 | 0.51911 |
| 6-pyruvoyl-tetrahydropterin synthase | BG072031 | −4.902929092 | 0.56213 |
| acetyl-Coenzyme A dehydrogenase, long-chain | BG066557 | −9.090909676 | 0.40106 |
| acetyl-Coenzyme A dehydrogenase, medium chain | AI840666 | −8.398490697 | 0.43686 |
| acyl-Coenzyme A dehydrogenase, very long chain | AI839605 | −6.18762928 | 0.59203 |
| acylphosphatase 2, muscle type | AA120674 | −7.657983239 | 0.33130 |
| adaptor-related protein complex AP-4, sigma 1 | BG069322 | −4.138928716 | 0.48502 |
| adenylate cyclase 6 | AA727732 | −5.870740066 | 0.47590 |
| ADP-ribosylation-like 3 | AV134034 | −4.98247219 | 0.45712 |
| ADP-ribosylation-like 4 | AA003086 | −4.452096978 | 0.45981 |
| adrenergic receptor kinase, beta 1 | BG072616 | −5.951311824 | 0.60538 |
| aldo-keto reductase family 1, member B3 (aldose reductase) | AV133992 | −5.029352566 | 0.74821 |
| aminolevulinate, delta-, dehydratase | BG063937 | −4.245991722 | 0.51637 |
| amino-terminal enhancer of split | AA968065 | −4.942847825 | 0.72701 |
| angiopoietin | BF538875 | −4.881730093 | 0.32339 |
| apoptotic chromatin condensation inducer in the nucleus | BG071714 | −4.62623729 | 0.47419 |
| ATP synthase, H+ transporting mitochondrial F1 complex, beta subunit [symbol] | AV006369 | −4.695530788 | 0.53925 |
| ATP synthase, H+ transporting, mitochondrial F0 complex, subunit b, is [symbol] | AI836064 | −6.423143997 | 0.45158 |
| ATP synthase, H+ transporting, mitochondrial F0 complex, subunit c (s [symbol] | AV095153 | −7.430215562 | 0.48878 |
| ATP synthase, H+ transporting, mitochondrial F0 complex, subunit c (s [symbol] | AV056821 | −4.424102615 | 0.52819 |
| ATP synthase, H+ transporting, mitochondrial F0 complex, subunit f, is [symbol] | BG073062 | −4.492001119 | 0.50909 |
| ATP synthase, H+ transporting, mitochondrial F0 complex, subunit g | BG069449 | −6.684865638 | 0.39574 |
| ATP synthase, H+ transporting, mitochondrial F1 complex, gamma pol [symbol] | BG072870 | −5.347883074 | 0.52850 |
| ATP synthase, H+ transporting, mitochondrial F1 complex, O subunit | AV133927 | −5.352698253 | 0.47237 |
| ATP synthase, H+ transporting, mitochondrial F1F0 complex, subunit [symbol] | BG072635 | −4.819618354 | 0.41437 |
| ATPase, Ca++ transporting, cardiac muscle, slow twitch 2 | AI837797 | −5.834521502 | 0.53249 |
| ATPase, H+ transporting, lysosomal 70 kD, V1 subunit A, isoform 1 | AW545296 | −4.280719124 | 0.75002 |
| AU RNA binding protein/enoyl-coenzyme A hydratase | AV095181 | −8.782972174 | 0.53747 |
| baculoviral IAP repeat-containing 4 | AV073504 | −5.130039053 | 0.68359 |
| bromodomain-containing 4 | AV085802 | −5.786610727 | 0.71518 |
| cadherin EGE LAG seven-pass G-type receptor 2 | BG074441 | −4.154879365 | 0.71952 |
| calcyclin binding protein | BG069742 | −8.690706344 | 0.65713 |
| capping protein alpha 3 | AV039134 | −5.081582357 | 0.42546 |
| carbonic anhydrase 14 | AV014385 | −5.82139814 | 0.40180 |
| carbonyl reductase 1 | AI323923 | −5.260736815 | 0.63722 |
| carboxylesterase 3 | BG072503 | −9.855339495 | 0.17436 |
| cardiac Abnormality/abnormal facies (CATCH22), microdeletion syndrc [symbol] | AV041840 | −9.98418961 | 0.40426 |
| carnitine palmitoyltransferase 2 | AV006197 | −5.312556125 | 0.62582 |
| caspase 1 | AA672522 | −5.482885752 | 0.50832 |
| caspase 14 | AJ007750 | −4.270794528 | 0.59138 |
| catenin src | C77281 | −5.060897945 | 0.55404 |
| cathepsin F | AV085152 | −5.325513355 | 0.51925 |
| Cbp/p300-interacting transactivator, with Glu/Asp-rich carboxy-termina [symbol] | BG069399 | −4.222038294 | 0.49555 |
| CDC-like kinase | BG065099 | −4.390363621 | 0.71405 |
| cell division cycle 5-like (S. pombe) | BG069455 | −4.117820871 | 0.62771 |
| citrate lyase beta like | AV028854 | −4.199225491 | 0.53480 |
| cleavage and polyadenylation specific factor 2, 100 kD subunit | AV111435 | −4.800913152 | 0.49169 |
| coagulation factor III | AA879919 | −6.686739114 | 0.58633 |
| cold inducible RNA binding protein | BG073558 | −14.8302043 | 0.37969 |
| complexin 2 | AV149907 | −4.775702769 | 0.37946 |
| copper chaperone for superoxide dismutase | AV093569 | −5.248357511 | 0.59552 |
| cornichon-like (Drosophila) | AV150049 | −5.432444546 | 0.56343 |
| creatine kinase, mitochondrial 2 | AV085004 | −4.742066271 | 0.61057 |
| cysteine-rich protein 3 | AV087451 | −4.266568219 | 0.39188 |
| cytochrome c oxidase subunit VIIb | AV093625 | −8.988138804 | 0.39401 |
| cytochrome c oxidase, subunit IVa | AV005997 | −4.487420289 | 0.41076 |
| cytochrome c oxidase, subunit Vb | AV088644 | −4.949569116 | 0.46997 |
| cytochrome c oxidase, subunit VI a, polypeptide 2 | AV001082 | −4.842370725 | 0.31139 |
| cytochrome c oxidase, subunit VI a, polypeptide 2 | AV030529 | −4.152568557 | 0.33572 |
| cytochrome c oxidase, subunit VIc | AV149855 | −9.192827977 | 0.37223 |
| cytochrome c oxidase, subunit VIIa 1 | AV086493 | −4.364923988 | 0.27457 |
| cytochrome c oxidase, subunit VIIa 3 | AV133935 | −5.936847157 | 0.47440 |
| cytochrome c oxidase, subunit VIIa 3 | BG072912 | −4.12193731 | 0.53257 |
| cytochrome c oxidase, subunit VIIc | BG063960 | −5.099803728 | 0.37129 |
| cytochrome c oxidase, subunit XVII assembly protein homolog (yeast) | AV081105 | −7.938746128 | 0.46201 |
| cytochrome c, somatic | AV086888 | −5.722105998 | 0.42669 |
| cytochrome c-1 | AV093672 | −5.446589149 | 0.68598 |
| cytochrome P450, 17 | AV042908 | −4.426517275 | 0.37805 |
| DEAD/H (Asp-Glu-Ala-Asp/His) box polypeptide 13 (RNA helicase A) | AV106868 | −6.374954218 | 0.67058 |
| DEAD/H (Asp-Glu-Ala-Asp/His) box polypeptide 20 | BG071005 | −4.145761402 | 0.69357 |
| death associated protein 3 | BG065205 | −6.784949232 | 0.48820 |
| deleted in polyposis 1 | AA032557 | −4.19567949 | 0.40696 |
| desmocollin 2 | BG063370 | −6.637675079 | 0.34694 |
| diacylglycerol kinase, alpha (80 kDa) | AV069373 | −4.808213153 | 0.58075 |
| diacylglycerol O-acyltransferase 2 | BG072524 | −5.216696741 | 0.26003 |
| diaphanous homolog 1 (Drosophila) | AV134828 | −4.349910406 | 0.64965 |
| DiGeorge syndrome critical region gene 6 | BG071919 | −4.99953028 | 0.52770 |
| dipeptidylpeptidase 4 | AA266854 | −5.003475925 | 0.66937 |
| DNA fragmentation factor, 40 kD, beta subunit | AV109088 | −4.25080084 | 0.65806 |
| DNA primase, p49 subunit | AV113083 | −9.821814843 | 0.49491 |
| DNA segment, Chr 14, ERATO Doi 574, expressed | BG068808 | −7.416007266 | 0.52173 |
| DNA segment, Chr 9, Wayne State University 149, expressed | AV135842 | −4.165273935 | 0.56300 |
| DnaJ (Hsp40) homolog, subfamily A, member 3 | AW540988 | −6.542750844 | 0.45648 |
| DnaJ (Hsp40) homolog, subfamily A, member 3 | AV050059 | −6.311708326 | 0.48336 |
| DnaJ (Hsp40) homolog, subfamily B, member 9 | AV041142 | −4.594900976 | 0.65180 |
| DnaJ (Hsp40) homolog, subfamily C, member 1 | AV057225 | −5.477300649 | 0.51634 |
| dodecenoyl-Coenzyme A delta isomerase (3,2 trans-enoyl-Coenyme A [symbol] | AA108563 | −7.017480503 | 0.35225 |
| down-regulated by Ctnnb1, a | BG068535 | −4.586302098 | 0.59629 |
| dynein, axon, heavy chain 11 | AA039110 | −4.619323446 | 0.41136 |
| dystonin | BG070533 | −4.583900131 | 0.55822 |
| dystroglycan 1 | BE137475 | −4.960612662 | 0.55724 |
| E2F transcription factor 6 | AV126035 | −4.440266193 | 0.57132 |
| ectodermal-neural cortex 1 | BG065122 | −5.705275017 | 0.55060 |
| endothelial monocyte activating polypeptide 2 | BG076119 | −4.974086698 | 0.59151 |
| endothelin 1 | AA511462 | −4.919891156 | 0.50725 |
| enigma homolog (R. norvegicus) | AV086590 | −4.495935882 | 0.46027 |
| enoyl coenzyme A hydratase 1, peroxisomal | BG074113 | −6.80582581 | 0.36476 |
| Eph receptor A4 | AV089919 | −4.344159052 | 0.34405 |
| ephrin A2 | AA036231 | −5.071477425 | 0.55979 |
| EST | AV084337 | −15.84609455 | 0.22443 |
| EST | AV089256 | −7.821945704 | 0.32354 |
| EST | AV088222 | −6.000803756 | 0.34203 |
| EST | BG067237 | −5.60660002 | 0.37931 |
| EST | AV092327 | −10.7313156 | 0.40744 |
| EST | BG067593 | −5.308733795 | 0.40771 |
| EST | AV104735 | −4.234815034 | 0.41649 |
| EST | AV107204 | −4.79899725 | 0.41907 |
| EST | AV090230 | −4.529261068 | 0.42529 |
| EST | AV032077 | −5.739628612 | 0.44260 |
| EST | BI076847 | −5.256943225 | 0.44584 |
| EST | BG066574 | −7.127384551 | 0.45000 |
| EST | AW558245 | −5.478409371 | 0.45389 |
| EST | AV089999 | −5.190665501 | 0.45408 |
| EST | AW554432 | −5.896214411 | 0.46163 |
| EST | AV006409 | −5.964082052 | 0.46864 |
| EST | AV058135 | −4.521649529 | 0.47454 |
| EST | AI836950 | −5.937211188 | 0.47461 |
| EST | AV092810 | −5.241936126 | 0.47602 |
| EST | AV112960 | −4.617628152 | 0.47834 |
| EST | AW545825 | −6.727669546 | 0.48212 |
| EST | AV085516 | −4.842648477 | 0.48488 |
| EST | AW538191 | −5.153458917 | 0.48631 |
| EST | AU024393 | −4.895288583 | 0.49035 |
| EST | AI836065 | −4.7755092 | 0.49306 |
| EST | AA855859 | −4.331305958 | 0.50195 |
| EST | BG068314 | −5.199228334 | 0.50230 |
| EST | AV043406 | −6.09893817 | 0.51042 |
| EST | AV066234 | −4.254484662 | 0.51985 |
| EST | AW537378 | −4.704989436 | 0.52235 |
| EST | BI076614 | −5.172671539 | 0.52412 |
| EST | C78728 | −4.342469046 | 0.52937 |
| EST | AV106287 | −4.157198249 | 0.53067 |
| EST | AV084802 | −5.166639576 | 0.53424 |
| EST | AV113584 | −5.364282201 | 0.53477 |
| EST | AV073557 | −4.506325346 | 0.54223 |
| EST | AV058085 | −8.095910962 | 0.54278 |
| EST | AV087849 | −6.671209615 | 0.54694 |
| EST | AV087838 | −8.769144558 | 0.54700 |
| EST | AV113429 | −6.64494074 | 0.54723 |
| EST | AI854089 | −4.234523551 | 0.55638 |
| EST | AW539454 | −4.298537333 | 0.56091 |
| EST | AV054545 | −6.94654287 | 0.56151 |
| EST | BG065742 | −13.00933301 | 0.56794 |
| EST | BG067648 | −8.683396149 | 0.57773 |
| EST | AW537634 | −5.324519908 | 0.57869 |
| EST | AW538620 | −5.025049378 | 0.58142 |
| EST | AW554258 | −5.832400646 | 0.59289 |
| EST | AW558391 | −4.257365597 | 0.59868 |
| EST | AV065563 | −4.768348545 | 0.60682 |
| EST | AW542440 | −4.491683933 | 0.62565 |
| EST | AW558803 | −5.020329084 | 0.63071 |
| EST | AW558059 | −4.281910751 | 0.63476 |
| EST | BG067262 | −5.922809848 | 0.63861 |
| EST | AW556930 | −4.246241225 | 0.65183 |
| EST | BG069129 | −4.137277132 | 0.66716 |
| EST | BG068320 | −4.21521866 | 0.67052 |
| EST | BG063124 | −4.343859108 | 0.67655 |
| EST | AV124902 | −6.244482147 | 0.68098 |
| EST | AV066141 | −4.258530103 | 0.70579 |
| EST | AW546201 | −5.334334206 | 0.71851 |
| ESTs | AV013380 | −8.675110287 | 0.12285 |
| ESTs | AI839959 | −11.80827248 | 0.26051 |
| ESTs | AV087279 | −10.84738974 | 0.37033 |
| ESTs | BG074584 | −4.991848058 | 0.41016 |
| ESTs | BG071766 | −7.140449539 | 0.41412 |
| ESTs | BG064317 | −5.723777122 | 0.42958 |
| ESTs | BG071847 | −5.928135678 | 0.43532 |
| ESTs | AW558570 | −4.480154195 | 0.45840 |
| ESTs | BG069296 | −5.240917448 | 0.46577 |
| ESTs | AV028938 | −4.151541241 | 0.48718 |
| ESTs | AI840562 | −12.06683549 | 0.49094 |
| ESTs | AV026027 | −4.506939508 | 0.49232 |
| ESTs | AV006522 | −4.613819892 | 0.52324 |
| ESTs | AV083513 | −4.828251577 | 0.53129 |
| ESTs | BG073031 | −4.566306264 | 0.53403 |
| ESTs | BG075173 | −5.028506537 | 0.53874 |
| ESTs | BG063906 | −8.089370979 | 0.54039 |
| ESTs | BG066954 | −4.782615457 | 0.54260 |
| ESTs | BG067242 | −6.82332378 | 0.54553 |
| ESTs | BG072934 | −5.228313195 | 0.54677 |
| ESTs | AI854088 | −4.159598239 | 0.55320 |
| ESTs | BG073667 | −10.48492722 | 0.55826 |
| ESTs | BG065948 | −4.860061653 | 0.56492 |
| ESTs | AV031990 | −6.549327409 | 0.56848 |
| ESTs | BG067986 | −7.07452791 | 0.58210 |
| ESTs | BG067553 | −5.000443636 | 0.59575 |
| ESTs | AV033253 | −4.213052314 | 0.59746 |
| ESTs | BG066080 | −7.178865626 | 0.60242 |
| ESTs | AV094549 | −5.448465601 | 0.61795 |
| ESTs | BG069475 | −5.197976115 | 0.63287 |
| ESTs | BG073483 | −5.580896625 | 0.63556 |
| ESTs | AU043006 | −6.902027048 | 0.63790 |
| ESTs | AW557124 | −4.400332672 | 0.67259 |
| ESTs | BG071818 | −6.164734724 | 0.67323 |
| ESTs | AV087922 | −5.463551198 | 0.68467 |
| ESTs | BG073793 | −5.556289784 | 0.69451 |
| ESTs | AV029719 | −4.64572808 | 0.70854 |
| ESTs | AU040991 | −4.656330027 | 0.71007 |
| ESTs | AV123079 | −4.487953887 | 0.79323 |
| ESTs | AA219953 | −4.928476302 | 0.81818 |
| ESTs, Highly similar to NUMM MOUSE NADH-UBIQUINONE OXIDOR [symbol] | AV053614 | −4.892019315 | 0.42037 |
| ESTs, Highly similar to SR68_HUMAN SIGNAL RECOGNITION PART [symbol] | AA044456 | −5.779140415 | 0.63127 |
| ESTs, Moderately similar to CENC MOUSE CENTROMERE PROTEIN [symbol] | BG070887 | −6.937133122 | 0.49208 |
| ESTs, Moderately similar to COXM MOUSE CYTOCHROME C OXIDA [symbol] | BG073133 | −4.382614329 | 0.38552 |
| ESTs, Moderately similar to hypothetical protein MGC2217 [Homo sap [symbol] | AV140202 | −5.884098532 | 0.42443 |
| ESTs, Moderately similar to put. gag and pol gene product [M. musculu [symbol] | AU017598 | −4.66917538 | 0.61340 |
| ESTs, Moderately similar to T29098 microtubule-associated protein 4, | AV085051 | −4.652120447 | 0.41777 |
| ESTs, Moderately similar to TSC1_RAT HAMARTIN (TUBEROUS SCI [symbol] | BG073522 | −4.528364031 | 0.57654 |
| ESTs, Moderately similar to unnamed protein product [H. sapiens] | BG069242 | −5.864025522 | 0.48855 |
| ESTs, Weakly similar to 17-beta hydroxysteroid dehydrogenase type 2 | AV012778 | −5.99546057 | 0.29569 |
| ESTs, Weakly similar to A48133 pre-mRNA splicing SRp75 [H. sapiens [symbol] | BG068996 | −8.42767335 | 0.41807 |
| ESTs, Weakly similar to COXD MOUSE CYTOCHROME C OXIDASE | AV088683 | −4.686650535 | 0.38315 |
| ESTs, Weakly similar to DIA3_MOUSE Diaphanous protein homolog 3 | BG066491 | −5.603551357 | 0.42357 |
| ESTs, Weakly similar to F-actin binding protein b-Nexilin [R. norvegicus [symbol] | AU022020 | −5.030069452 | 0.55649 |
| ESTs, Weakly similar to FOR4 MOUSE FORMIN 4 [M. musculus] | BG068457 | −5.127410189 | 0.51270 |
| ESTs, Weakly similar to proline rich protein 2 [Mus musculus] [M. musc [symbol] | BG068802 | −6.578307544 | 0.63820 |
| ESTs, Weakly similar to S33477 hypothetical protein 1 —rat [R. norvegi [symbol] | BG063187 | −4.666226794 | 0.59621 |
| ESTs, Weakly similar to S48081 GRSF-1 protein [H. sapiens] | AV074326 | −4.328278109 | 0.58441 |
| ESTs, Weakly similar to SNAP190 [H. sapiens] | AV094673 | −4.368590902 | 0.62151 |
| ESTs, Weakly similar to testis derived transcript 3 [Mus musculus] [M. r [symbol] | BG065317 | −5.144519948 | 0.39289 |
| ESTs, Weakly similar to TLM MOUSE TLM PROTEIN [M. musculus] | AV092958 | −6.150403741 | 0.45074 |
| eukaryotic translation elongation factor 1 delta (guanine nucleotide exc [symbol] | AA253918 | −4.186569986 | 0.57143 |
| eukaryotic translation elongation factor 2 | BG067570 | −6.371044444 | 0.65020 |
| eukaryotic translation initiation factor 2 alpha kinase 3 | AV095205 | −5.059393319 | 0.56401 |
| eukaryotic translation initiation factor 3, subunit 2 (beta, 36 kD) | AV094437 | −4.601527312 | 0.45547 |
| excision repair cross-complementing rodent repair deficiency, complen [symbol] | BG063161 | −5.547050872 | 0.63136 |
| expressed sequence AA407270 | BG063148 | −5.93566094 | 0.40575 |
| expressed sequence AA407270 | AV024203 | −5.771368225 | 0.55519 |
| expressed sequence AA408168 | BG066580 | −7.720142458 | 0.42173 |
| expressed sequence AA408877 | AV009485 | −7.331843342 | 0.44266 |
| expressed sequence AA408877 | BG063884 | −7.549736289 | 0.69757 |
| expressed sequence AA959758 | BG070652 | −6.210569504 | 0.69281 |
| expressed sequence AA959857 | AV109470 | −6.111199231 | 0.57250 |
| expressed sequence AA960047 | AV033573 | −4.632811011 | 0.71552 |
| expressed sequence AI197390 | BG064453 | −4.447429392 | 0.65801 |
| expressed sequence AI256693 | AV083357 | −7.061594227 | 0.44924 |
| expressed sequence AI256693 | BG062933 | −6.84069401 | 0.50397 |
| expressed sequence AI314967 | BG075147 | −9.700426666 | 0.58836 |
| expressed sequence AI315037 | AV014911 | −4.168917128 | 0.46734 |
| expressed sequence AI414265 | BG063334 | −5.374078873 | 0.35065 |
| expressed sequence AI428506 | AV032231 | −4.312084153 | 0.46225 |
| expressed sequence AI428794 | BG076075 | −4.228379709 | 0.69144 |
| expressed sequence AI450287 | BG065344 | −6.167875756 | 0.74403 |
| expressed sequence AI451892 | AV032341 | −4.405035852 | 0.58191 |
| expressed sequence AI452301 | BI076508 | −8.197208043 | 0.54245 |
| expressed sequence AI462702 | BG068253 | −6.418310883 | 0.57868 |
| expressed sequence AI480535 | AV083879 | −5.187049508 | 0.47634 |
| expressed sequence AI504630 | AV015284 | −5.888394236 | 0.56047 |
| expressed sequence AI595366 | AV086025 | −7.209264922 | 0.54969 |
| expressed sequence AI604911 | BG063457 | −6.27869333 | 0.60458 |
| expressed sequence AI746547 | BG073543 | −4.303474374 | 0.66202 |
| expressed sequence AI838773 | AV013448 | −5.430320297 | 0.51111 |
| expressed sequence AU022809 | AU022809 | −6.877820253 | 0.37946 |
| expressed sequence AU040217 | AV006387 | −4.601437144 | 0.37921 |
| expressed sequence AU043990 | AV085893 | −4.61060875 | 0.61610 |
| expressed sequence AV006127 | AV006127 | −4.968478814 | 0.55637 |
| expressed sequence AV028368 | AV010507 | −4.92003212 | 0.42417 |
| expressed sequence AW122032 | BG071778 | −5.449835828 | 0.53237 |
| expressed sequence AW125446 | BG070892 | −6.504525167 | 0.53458 |
| expressed sequence AW215868 | BG069736 | −4.284651389 | 0.71600 |
| expressed sequence AW495846 | BG076492 | −4.461876137 | 0.66865 |
| expressed sequence AW545363 | AV060425 | −4.699771388 | 0.68385 |
| expressed sequence AW554339 | AW554339 | −4.990896506 | 0.68667 |
| expressed sequence AW555814 | BG065375 | −5.729264312 | 0.37042 |
| expressed sequence C76711 | C76711 | −4.673701033 | 0.54362 |
| expressed sequence C78643 | C78643 | −4.923270952 | 0.57835 |
| expressed sequence C79026 | BG066389 | −4.28748357 | 0.68151 |
| expressed sequence C81189 | BG066971 | −5.597395275 | 0.41821 |
| expressed sequence C85317 | BG067152 | −5.135834608 | 0.52423 |
| expressed sequence C86676 | BG069605 | −5.566957046 | 0.59228 |
| expressed sequence C87882 | BG067895 | −5.351181214 | 0.51928 |
| expressed sequence R74645 | AV032243 | −4.837023248 | 0.46405 |
| Fas-activated serine/threonine kinase | BG074856 | −4.217025613 | 0.45434 |
| fatty acid binding protein 3, muscle and heart | AV006024 | −7.308756431 | 0.40356 |
| fatty acid Coenzyme A ligase, long chain 2 | AV006061 | −4.941866769 | 0.48297 |
| FBJ osteosarcoma oncogene B | BG076079 | −7.042746377 | 0.52580 |
| f-box and leucine-rich repeat protein 12 | BG067545 | −4.400264381 | 0.77610 |
| fibroblast growth factor receptor 4 | AI385693 | −5.90785626 | 0.48522 |
| FK506 binding protein 3 (25 kD) | AV134155 | −12.24059879 | 0.46456 |
| forkhead box C1 | A1415347 | −4.299584893 | 0.64530 |
| four and a half LIM domains 2 | BG065614 | −4.837322463 | 0.40643 |
| G protein-coupled receptor kinase 7 | AV005838 | −5.282517048 | 0.50864 |
| galactokinase | AV108357 | −4.391030016 | 0.47824 |
| gamma-glutamyl transpeptidase | AA162908 | −4.562953433 | 0.41377 |
| gelsolin | AV170949 | −7.811644475 | 0.39819 |
| gene rich cluster, C8 gene | C81126 | −7.15072821 | 0.68777 |
| genes associated with retinoid-IFN-induced mortality 19 | BG073545 | −6.967346166 | 0.40268 |
| glioblastoma amplified sequence | AV082190 | −7.336574711 | 0.44947 |
| glucocorticoid-induced leucine zipper | W33468 | −4.377977394 | 0.39408 |
| glutamate oxaloacetate transaminase 1, soluble | BG066689 | −5.113196958 | 0.41673 |
| glutamine synthetase | AV009064 | −5.494322506 | 0.38899 |
| glutathione S-transferase, alpha 4 | AV084880 | −5.620268508 | 0.49942 |
| glutathione S-transferase, mu 1 | BG074268 | −4.904981635 | 0.48909 |
| glycosylphosphatidylinositol specific phospholipase D1 | AV086924 | −6.085890514 | 0.44720 |
| granzyme B | AV038272 | −4.606881006 | 0.42438 |
| growth factor receptor bound protein 2-associated protein 1 | BG063323 | −4.173021249 | 0.73731 |
| guanosine monophosphate reductase | AV103032 | −4.121459006 | 0.49495 |
| H2A histone family, member Y | C75971 | −9.632930002 | 0.29998 |
| heat shock 10 kDa protein 1 (chaperonin 10) | AV055529 | −4.14388602 | 0.66410 |
| heat shock protein, 70 kDa 3 | AV223941 | −4.717867523 | 0.42727 |
| heme oxygenase (decycling) 1 | AV083964 | −9.130108662 | 0.57613 |
| hemoglobin, beta adult major chain | AV108710 | −6.575328842 | 0.48588 |
| histidine ammonia lyase | AV022721 | −5.357960558 | 0.44637 |
| histidine rich calcium binding protein | BG073810 | −7.723374649 | 0.29908 |
| histidine triad nucleotide binding protein | AA154889 | −4.936798282 | 0.68692 |
| histocompatibility 47 | AV036651 | −7.347503305 | 0.63359 |
| homeo box C4 | AA245472 | −4.46392246 | 0.41142 |
| homocysteine-inducible, endoplasmic reticulum stress-inducible, ubiqu [symbol] | AV086303 | −4.450795031 | 0.32623 |
| hydroxysteroid (17-beta) dehydrogenase 10 | BG073539 | −5.757417226 | 0.49471 |
| hypothetical protein, MGC: 6943 | AV085351 | −4.547811108 | 0.62294 |
| hypothetical protein, MGC: 6989 | AV031846 | −4.932452886 | 0.38973 |
| hypothetical protein, MGC: 7550 | AV087882 | −8.375970889 | 0.61973 |
| immediate early responses 5 | BG069628 | −4.158460406 | 0.56982 |
| immunoglobulin superfamily, member 7 | AV073565 | −7.864977871 | 0.52541 |
| insulin-like growth factor binding protein 4 | AV005795 | −5.368416582 | 0.18068 |
| insulin-like growth factor binding protein 5 | AV087798 | −6.367247348 | 0.43614 |
| integrin binding sialoprotein | AV171934 | −4.99290928 | 0.34304 |
| interferon activated gene 204 | AV015208 | −7.701331319 | 0.64560 |
| interferon activated gene 205 | AV058630 | −8.015190946 | 0.34982 |
| interferon-related developmental regulator 1 | AA107115 | −4.366931288 | 0.67719 |
| iroquois related homeobox 4 (Drosophila) | AV006035 | −6.23099642 | 0.58603 |
| isocitrate dehydrogenase 2 (NADP+), mitochondrial | AV089252 | −5.278687285 | 0.45360 |
| isocitrate dehydrogenase 3 (NAD+) alpha | BG068774 | −4.55487821 | 0.45957 |
| isocitrate dehydrogenase 3 (NAD+) beta | AA036340 | −4.162269318 | 0.47460 |
| isovaleryl coenzyme A dehydrogenase | BG070984 | −8.767935605 | 0.30518 |
| Janus kinase 1 | BG067874 | −7.25451775 | 0.65078 |
| Janus kinase 2 | AA153109 | −5.307586645 | 0.64858 |
| keratin associated protein 6-2 | AV013499 | −5.525131815 | 0.38744 |
| keratin complex 2, basic, gene 16 | AA738772 | −4.266087447 | 0.51812 |
| keratin complex 2, basic, gene 18 | AV086522 | −4.989188404 | 0.40787 |
| keratin complex 2, basic, gene 6g | AV008410 | −5.481104059 | 0.33635 |
| L-3-hydroxyacyl-Coenzyme A dehydrogenase, short chain | AA122758 | −7.489259426 | 0.44349 |
| lactate dehydrogenase 2, B chain | AV171750 | −4.652580719 | 0.33146 |
| leucine zipper-EF-hand containing transmembrane protein 1 | AV083103 | −4.847170719 | 0.65147 |
| LIM domain binding 3 | AV088371 | −4.401196368 | 0.41447 |
| lipin 1 | AV022047 | −4.914016394 | 0.52166 |
| lipoprotein lipase | AV084650 | −4.839334145 | 0.42555 |
| lipoprotein lipase | AV006290 | −11.42464459 | 0.42847 |
| low density lipoprotein receptor-related protein 2 | BG064854 | −4.220186803 | 0.59503 |
| lurcher transcript 1 | BG074415 | −6.244274361 | 0.41951 |
| lysosomal apyrase-like 1 | AV086322 | −6.775781299 | 0.65322 |
| lysosomal membrane glycoprotein 2 | BG074453 | −6.248153587 | 0.74154 |
| malate dehydrogenase, soluble | AV093576 | −5.202957456 | 0.32039 |
| MAP kinase-activated protein kinase 2 | AA030342 | −7.597964206 | 0.59516 |
| MAP kinase-activated protein kinase 5 | AA616241 | −6.281175594 | 0.51661 |
| maternal embryonic leucine zipper kinase | AV140411 | −5.56058333 | 0.51604 |
| membrane-associated protein 17 | AV060358 | −4.806294256 | 0.39397 |
| methyl-CpG binding domain protein 4 | AV032932 | −4.628918539 | 0.55652 |
| methylmalonyl-Coenzyme A mutase | AV031545 | −5.467911803 | 0.50168 |
| microsomal glutathione S-transferase 3 | AV056432 | −4.333591334 | 0.41688 |
| microtubule-associated protein tau | BG066372 | −4.116954726 | 0.42329 |
| mitochondrial ribosomal protein 64 | AV094889 | −4.490503004 | 0.63412 |
| mitochondrial ribosomal protein L15 | BG064987 | −5.229142603 | 0.54936 |
| mitochondrial ribosomal protein L16 | BG075780 | −4.148872464 | 0.60350 |
| mitochondrial ribosomal protein L23 | BG071604 | −7.059249111 | 0.49751 |
| mitochondrial ribosomal protein L39 | AV150063 | −6.943179503 | 0.67150 |
| mitochondrial ribosomal protein L43 | AV094774 | −4.968939433 | 0.69126 |
| mitochondrial ribosomal protein S17 | BG071752 | −5.227257781 | 0.42507 |
| mitochondrial ribosomal protein S25 | BG065867 | −6.463001045 | 0.47504 |
| mitochondrial ribosomal protein S31 | AV058185 | −4.943328985 | 0.52131 |
| mitogen activated protein binding protein interacting protein | AV134069 | −5.084504328 | 0.63511 |
| mitogen-activated protein kinase kinase kinase 7 interacting protein 2 | AV011185 | −5.269766834 | 0.51165 |
| MLN51 protein | AW556296 | −6.239103687 | 0.56037 |
| Mus musculus 10 day old male pancreas cDNA, RIKEN full-length enri [symbol] | AV058496 | −9.867161529 | 0.43027 |
| Mus musculus 10, 11 days embryo whole body cDNA, RIKEN full-leng [symbol] | BG075565 | −6.173663343 | 0.72665 |
| Mus musculus brain and reproductive organ-expressed protein (Bre) m [symbol] | AV073509 | −4.883581812 | 0.51095 |
| Mus musculus methyl-CpG binding domain protein 3-like protein 2 (Mb [symbol] | BG071308 | −5.716981372 | 0.53500 |
| Mus musculus QIL1 (Qil1) mRNA, complete cds | BG072356 | −5.841602916 | 0.46840 |
| Mus musculus, clone IMAGE: 3491909, mRNA, partial cds | BG071756 | −4.496303875 | 0.65826 |
| Mus musculus, clone IMAGE: 4482598, mRNA | AA034560 | −4.150299072 | 0.31779 |
| Mus musculus, clone IMAGE: 5357662, mRNA, partial cds | AV042520 | −4.408584942 | 0.60396 |
| Mus musculus, clone MGC: 11691 IMAGE: 3962417, mRNA, complete [symbol] | AV084848 | −5.490316133 | 0.52085 |
| Mus musculus, clone MGC: 36369 IMAGE: 4982239, mRNA, complete [symbol] | AV094465 | −5.44774435 | 0.49239 |
| Mus musculus, clone MGC: 6816 IMAGE: 2648797, mRNA, complete c [symbol] | AV014114 | −4.282850534 | 0.53438 |
| Mus musculus, clone MGC: 7480 IMAGE: 3490700, mRNA, complete c [symbol] | AV034637 | −5.987456834 | 0.50215 |
| Mus musculus, clone MGC: 7530 IMAGE: 3492114, mRNA, complete c [symbol] | AV089939 | −6.833387684 | 0.58423 |
| Mus musculus, H4 histone family, member A, clone MGC: 30488 IMAG [symbol] | AV113959 | −4.622426446 | 0.45955 |
| Mus musculus, hypothetical protein MGC11287 similar to ribosomal p [symbol] | AV031726 | −5.584850445 | 0.70092 |
| Mus musculus, Similar to 3-hydroxyisobutyrate dehydrogenase, clone I | AI854120 | −5.249848661 | 0.50351 |
| Mus musculus, Similar to ATPase, Na+/K+ transporting, alpha 1a.1 po [symbol] | AA063844 | −4.712431921 | 0.52469 |
| Mus musculus, Similar to chromosome 18 open reading frame 1, clone [symbol] | BG070238 | −4.251926511 | 0.72193 |
| Mus musculus, Similar to electron-transfer-flavoprotein, alpha polypep [symbol] | AV088774 | −5.68750046 | 0.47951 |
| Mus musculus, Similar to glutamate rich WD repeat protein GRWD, c [symbol] | BG071389 | −4.464168152 | 0.69603 |
| Mus musculus, Similar to hypothetical protein BC004409, clone MGC: [symbol] | AV086576 | −5.211455456 | 0.54638 |
| Mus musculus, Similar to hypothetical protein MGC4368, clone MGC: 2 | BG065643 | −4.140909089 | 0.53064 |
| Mus musculus, Similar to hypothetical protein MGC4368, clone MGC: 2 | AV005807 | −4.448246934 | 0.54984 |
| Mus musculus, Similar to hypothetical protein, clone MGC: 19257 IMA [symbol] | AV055251 | −5.964031565 | 0.71353 |
| Mus musculus, Similar to mannosyl (alpha-1,3-)-glycoprotein beta-1,4- [symbol] | BG063179 | −4.963893564 | 0.68444 |
| Mus musculus, Similar to metallothionein 1, clone MGC: 27821 IMAGE: [symbol] | AV149953 | −5.009409882 | 0.38263 |
| Mus musculus, Similar to MIPP65 protein, clone MGC: 18783 IMAGE: 4 | AV109599 | −4.769020513 | 0.62297 |
| Mus musculus, Similar to PTD015 protein, clone MGC: 36240 IMAGE: 5 | AV088778 | −4.30312782 | 0.51111 |
| Mus musculus, Similar to secretory leukocyte protease inhibitor, clone | AV089194 | −5.393553048 | 0.56725 |
| Mus musculus, Similar to transmembrane protein 5, clone MGC: 28135 [symbol] | AV095048 | −4.755442646 | 0.65205 |
| myeloblastosis oncogene | AV222464 | −5.594373043 | 0.63770 |
| myeloid leukemia factor 1 | AV042698 | −6.286060346 | 0.36555 |
| myosin binding protein C, cardiac | AV005840 | −4.40479052 | 0.56183 |
| myosin light chain, alkali, cardiac atria | AV005821 | −7.047964424 | 0.31699 |
| N-acetyltransferase ARD1 homolog (S. cerevisiae) | AI841645 | −4.230855583 | 0.72328 |
| NADH dehydrogenase (ubiquinone) 1 alpha subcomplex 2 | AV016078 | −6.793461475 | 0.40427 |
| NADH dehydrogenase (ubiquinone) 1 alpha subcomplex 2 | AV093541 | −5.380207421 | 0.51264 |
| NADH dehydrogenase (ubiquinone) 1 alpha subcomplex, 1 | AV140287 | −7.671234989 | 0.49739 |
| NADH dehydrogenase (ubiquinone) 1 alpha subcomplex, 4 | AV050140 | −4.641798789 | 0.43550 |
| NADH dehydrogenase (ubiquinone) 1 alpha subcomplex, 6 (14 kD, B1 [symbol] | AV106199 | −5.540201021 | 0.41067 |
| NADH dehydrogenase (ubiquinone) 1 alpha subcomplex, 6 (14 kD, B1 [symbol] | AV087995 | −4.857759692 | 0.46752 |
| NADH dehydrogenase (ubiquinone) 1 alpha subcomplex, 7 (14.5 kD, B [symbol] | AV133797 | −4.463338846 | 0.45989 |
| NADH dehydrogenase (ubiquinone) 1 beta subcomplex 5 | AV057902 | −6.33345429 | 0.40844 |
| NADH dehydrogenase (ubiquinone) 1 beta subcomplex, 9 | BG075174 | −5.525039706 | 0.44325 |
| NADH dehydrogenase (ubiquinone) 1, subcomplex Unsequenced EST [symbol] | AV088122 | −4.47328854 | 0.43713 |
| NADH dehydrogenase (ubiquinone) Fe—S protein 3 | BG076060 | −7.829252699 | 0.40260 |
| NADH dehydrogenase (ubiquinone) Fe—S protein 4 | BG066265 | −4.786795598 | 0.56585 |
| nebulin-related anchoring protein | AV013274 | −4.709864985 | 0.31656 |
| neurotensin receptor 2 | AV032954 | −6.394790155 | 0.34827 |
| Niemann Pick type C1 | AV012796 | −5.818245482 | 0.57019 |
| N-myc downstream regulated 2 | AV149939 | −4.956548973 | 0.47960 |
| non MHC restricted killing associated | BG076189 | −5.906532297 | 0.56544 |
| N-sulfotransferase | AV051308 | −4.548362727 | 0.41566 |
| nuclear distribution gene C homolog (Aspergillus) | BG073422 | −10.8626569 | 0.56353 |
| nuclear receptor coactivator 6 interacting protein | AV113681 | −6.148669995 | 0.34592 |
| nuclear receptor interacting protein 1 | AI840578 | −4.612742367 | 0.59793 |
| nuclear receptor subfamily 2, group F, member 1 | BG071238 | −4.980625532 | 0.35648 |
| nuclear transcription factor-Y beta | AV016446 | −6.246444283 | 0.41297 |
| olfactomedin 1 | BG073096 | −7.286235688 | 0.39555 |
| oxysterol binding protein-like 1A | BG073162 | −6.812913131 | 0.57590 |
| p53 apoptosis effector related to Pmp22 | BG065306 | −4.678975404 | 0.40269 |
| p53 regulated PA26 nuclear protein | BG076140 | −5.448306149 | 0.55541 |
| paired box gene 6 | AV032892 | −4.488629951 | 0.61857 |
| pantophysin | AV091203 | −4.149100799 | 0.69535 |
| PCTAIRE-motif protein kinase 1 | AV157322 | −5.035290036 | 0.46140 |
| pellino 1 | BG063809 | −6.156617986 | 0.49251 |
| peptidase 4 | U51014 | −4.3323071 | 0.47568 |
| peptidylprolyl isomerase (cyclophilin)-like 1 | AV015645 | −4.821247351 | 0.32093 |
| periplakin | BG074644 | −4.757437218 | 0.33818 |
| peroxiredoxin 3 | AA168985 | −10.6903742 | 0.41739 |
| peroxiredoxin 6 | AV052763 | −4.530139145 | 0.54965 |
| peroxisomal membrane protein 2, 22 kDa | BG073687 | −5.266196231 | 0.36957 |
| peroxisomal membrane protein 3, 35 kDa | BG075110 | −4.851555962 | 0.58487 |
| peroxisome proliferative activated receptor, gamma, coactivator 1 | AF049330 | −5.741819935 | 0.48224 |
| phosphate cytidylyltransferase 1, choline, alpha isoform | BG071157 | −8.214581306 | 0.56759 |
| phosphatidylinositol 3 kinase, regulatory subunit, polypeptide 4, p150 | BG069962 | −5.634662461 | 0.72045 |
| phosphofructokinase, muscle | AV012100 | −4.863378338 | 0.31668 |
| phospholipase A2 group VII (platelet-activating factor acetylhydrolase, | AV033702 | −4.176805214 | 0.45211 |
| phospholipase A2, group IB, pancreas | AV085478 | −7.151034427 | 0.68461 |
| phosphoribosylglycinamide formyltransferase | AV009977 | −6.77843399 | 0.62257 |
| phytanoyl-CoA hydroxylase | AV084314 | −9.87801812 | 0.28442 |
| platelet-derived growth factor receptor-like | BG068957 | −5.060999551 | 0.39457 |
| polymyositis/scleroderma autoantigen 2 | BG063453 | −5.530726571 | 0.44618 |
| potassium voltage-gated channel, Shal-related family, member 2 | BG075283 | −4.752089401 | 0.48273 |
| pre-B-cell colony-enhancing factor | AV108470 | −4.183827947 | 0.53050 |
| prefoldin 2 | AU020724 | −6.551694173 | 0.50227 |
| pregnancy upregulated non-ubiquitously expressed CaM kinase | AI391204 | −4.976455425 | 0.67410 |
| programmed cell death 5 | BG063248 | −4.346750922 | 0.47631 |
| proteasome (prosome, macropain) 26S subunit, non-ATPase, 4 | AV111455 | −4.786266311 | 0.70045 |
| proteasome (prosome, macropain) subunit, alpha type 7 | AV093698 | −7.206924146 | 0.71542 |
| proteasome (prosome, macropain) subunit, beta type 6 | AV093807 | −4.135275065 | 0.73806 |
| protein kinase inhibitor, gamma | BG073627 | −5.407677293 | 0.66327 |
| protein kinase, AMP-activated, gamma 1 non-catalytic subunit | BG067722 | −5.174284179 | 0.48660 |
| protein phospatase 3, regulatory subunit B, alpha isoform (calcineurin [symbol] | AV006032 | −4.245876461 | 0.32451 |
| protein tyrosine phosphatase, non-receptor type 9 | AV114744 | −4.237859546 | 0.58064 |
| pyruvate dehydrogenase E1 alpha 1 | BG068736 | −6.333567491 | 0.40029 |
| quaking | BG068631 | −4.93071726 | 0.57698 |
| Rab acceptor 1 (prenylated) | BG072002 | −5.608012206 | 0.48144 |
| RAN guanine nucleotide release factor | AV133777 | −4.36279612 | 0.59926 |
| RAS-homolog enriched in brain | AV095119 | −4.879211565 | 0.53004 |
| RAS-related C3 botulinum substrate 1 | BG076502 | −6.040933852 | 0.60293 |
| receptor (calcitonin) activity modifying protein 2 | AV085507 | −5.303383378 | 0.54970 |
| receptor-associated protein of the synapse, 43 kDa | AV061434 | −10.61862114 | 0.41436 |
| regulator of G-protein signaling 2 | BG068533 | −4.835282956 | 0.27907 |
| reticulon 2 (Z-band associated protein) | AV088718 | −5.623316329 | 0.44935 |
| retinoic acid induced 1 | AV012729 | −4.290030308 | 0.63998 |
| retinoid X receptor gamma | AV089219 | −5.822213161 | 0.49561 |
| ribosomal protein L27a | AV013292 | −4.437253914 | 0.49756 |
| ribosomal protein L30 | BG065356 | −4.252974113 | 0.68577 |
| ribosomal protein L37a | AI837822 | −5.154049385 | 0.59292 |
| ribosomal protein S25 | AV093430 | −4.658335514 | 0.58295 |
| ribosomal protein S29 | L31609 | −6.110664766 | 0.45134 |
| RIKEN cDNA 0610006N12 gene | AA110681 | −6.75185087 | 0.40291 |
| RIKEN cDNA 0610007H07 gene | BG072309 | −4.126129022 | 0.60173 |
| RIKEN cDNA 0610009D10 gene | AA154397 | −7.08466256 | 0.34713 |
| RIKEN cDNA 0610009I16 gene | AV086609 | −7.236199669 | 0.35051 |
| RIKEN cDNA 0610010E03 gene | AI841340 | −6.802249485 | 0.47787 |
| RIKEN cDNA 0610010I17 gene | AV056903 | −5.538754596 | 0.46727 |
| RIKEN cDNA 0610010I23 gene | AV051596 | −4.328819955 | 0.61515 |
| RIKEN cDNA 0610011B04 gene | BG073700 | −6.555996854 | 0.38623 |
| RIKEN cDNA 0610011L04 gene | BG072552 | −5.054443334 | 0.37549 |
| RIKEN cDNA 0610025I19 gene | AV085433 | −17.56809908 | 0.22127 |
| RIKEN cDNA 0610033L03 gene | AV093484 | −7.039284704 | 0.41225 |
| RIKEN cDNA 0610039N19 gene | AV083519 | −5.406448324 | 0.41668 |
| RIKEN cDNA 0610039N19 gene | BG066600 | −5.330882468 | 0.45065 |
| RIKEN cDNA 0610040D20 gene | AV004247 | −4.512757398 | 0.63567 |
| RIKEN cDNA 0710008D09 gene | AW558029 | −4.729146692 | 0.46971 |
| RIKEN cDNA 1010001M12 gene | AV086467 | −7.48040813 | 0.44085 |
| RIKEN cDNA 1010001N11 gene | AV133828 | −4.686104019 | 0.46207 |
| RIKEN cDNA 1100001F19 gene | BG070073 | −5.288822697 | 0.68489 |
| RIKEN cDNA 1110001A12 gene | BG070781 | −4.703835715 | 0.64679 |
| RIKEN cDNA 1110001I24 gene | AV140151 | −6.052802797 | 0.36840 |
| RIKEN cDNA 1110001J03 gene | AV065564 | −4.192297591 | 0.32893 |
| RIKEN cDNA 1110001O19 gene | AV056481 | −4.314017396 | 0.56079 |
| RIKEN cDNA 1110003P16 gene | BG075816 | −4.46363954 | 0.51085 |
| RIKEN cDNA 1110003P16 gene | AV057754 | −4.970604264 | 0.55663 |
| RIKEN cDNA 1110004A22 gene | BG071279 | −4.457797204 | 0.48172 |
| RIKEN cDNA 1110007A04 gene | AV055217 | −4.969107085 | 0.47342 |
| RIKEN cDNA 1110007C09 gene | AV051158 | −4.118786157 | 0.53859 |
| RIKEN cDNA 1110008L20 gene | AV018091 | −4.697507959 | 0.52248 |
| RIKEN cDNA 1110013H04 gene | AV052337 | −6.788162338 | 0.45818 |
| RIKEN cDNA 1110013H04 gene | BG068276 | −6.06832892 | 0.56841 |
| RIKEN cDNA 1110018B13 gene | AV028535 | −4.615083855 | 0.43160 |
| RIKEN cDNA 1110018B13 gene | AV084595 | −5.97322181 | 0.57666 |
| RIKEN cDNA 1110020I04 gene | AV051530 | −14.92032087 | 0.30711 |
| RIKEN cDNA 1110020I04 gene | BG063739 | −4.463807689 | 0.47696 |
| RIKEN cDNA 1110020J08 gene | AW550860 | −4.614727887 | 0.61323 |
| RIKEN cDNA 1110021D01 gene | AV071376 | −4.58410245 | 0.79871 |
| RIKEN cDNA 1110028A07 gene | AV085772 | −6.174919065 | 0.39958 |
| RIKEN cDNA 1110031C13 gene | AV041472 | −5.028419389 | 0.46491 |
| RIKEN cDNA 1110031I02 gene | AU043030 | −4.403755369 | 0.51919 |
| RIKEN cDNA 1110036H21 gene | AV012479 | −5.160074727 | 0.45281 |
| RIKEN cDNA 1110054G21 gene | AV014368 | −5.027901058 | 0.49410 |
| RIKEN cDNA 1110063J16 gene | AV078407 | −5.999746891 | 0.59492 |
| RIKEN cDNA 1110065A22 gene | AV016366 | −4.92541762 | 0.51442 |
| RIKEN cDNA 1190002A23 gene | AV024081 | −5.535759516 | 0.60154 |
| RIKEN cDNA 1190002L16 gene | BG071000 | −6.490599379 | 0.52952 |
| RIKEN cDNA 1190006F07 gene | AI839764 | −6.766591842 | 0.28987 |
| RIKEN cDNA 1190006F07 gene | BG072458 | −4.615357067 | 0.47455 |
| RIKEN cDNA 1190006L01 gene | BG076352 | −6.238204432 | 0.38844 |
| RIKEN cDNA 1190017B19 gene | AV022384 | −4.286049069 | 0.61201 |
| RIKEN cDNA 1200006O19 gene | BG071963 | −4.904434126 | 0.49222 |
| RIKEN cDNA 1200006O19 gene | AV074439 | −4.359926363 | 0.57055 |
| RIKEN cDNA 1200007E24 gene | BG075635 | −5.547606302 | 0.54461 |
| RIKEN cDNA 1200009K13 gene | BG069392 | −4.497346028 | 0.66746 |
| RIKEN cDNA 1200015P04 gene | AV065655 | −6.152236946 | 0.15180 |
| RIKEN cDNA 1200015P04 gene | AV067337 | −8.636968452 | 0.18033 |
| RIKEN cDNA 1200015P04 gene | AI840878 | −8.089636915 | 0.18339 |
| RIKEN cDNA 1200015P04 gene | AV068725 | −9.796466054 | 0.22295 |
| RIKEN cDNA 1300002C13 gene | BG064110 | −6.428715365 | 0.48112 |
| RIKEN cDNA 1300013G12 gene | BG076497 | −6.939802129 | 0.53379 |
| RIKEN cDNA 1300013J15 gene | AV082636 | −4.431683442 | 0.42023 |
| RIKEN cDNA 1300017C12 gene | BG069813 | −5.158800113 | 0.47198 |
| RIKEN cDNA 1300019P08 gene | AV094927 | −6.036452338 | 0.46761 |
| RIKEN cDNA 1500001L03 gene | BG067671 | −4.740520776 | 0.33865 |
| RIKEN cDNA 1500004O06 gene | AV084141 | −10.93331411 | 0.53732 |
| RIKEN cDNA 1500004O06 gene | AV095102 | −4.337275885 | 0.59115 |
| RIKEN cDNA 1500010M16 gene | AV162350 | −4.399118243 | 0.53491 |
| RIKEN cDNA 1500012D08 gene | AV094880 | −5.354092617 | 0.47779 |
| RIKEN cDNA 1500032E05 gene | AI894110 | −5.272445403 | 0.58956 |
| RIKEN cDNA 1500034J20 gene | AV111483 | −8.495755577 | 0.49446 |
| RIKEN cDNA 1500036F01 gene | AV074483 | −4.169290222 | 0.23080 |
| RIKEN cDNA 1600014J01 gene | AV051090 | −6.532850795 | 0.57481 |
| RIKEN cDNA 1600023A02 gene | AV002462 | −4.735699762 | 0.55362 |
| RIKEN cDNA 1700006F03 gene | BG071686 | −6.491908138 | 0.57462 |
| RIKEN cDNA 1700013G20 gene | BG067233 | −5.577143706 | 0.50168 |
| RIKEN cDNA 1700016D08 gene | BG073980 | −4.295578649 | 0.66457 |
| RIKEN cDNA 1700029P11 gene | AV043746 | −4.981358021 | 0.38488 |
| RIKEN cDNA 1700029P11 gene | AV043137 | −8.428540481 | 0.48877 |
| RIKEN cDNA 1810004I06 gene | AV050264 | −5.021183923 | 0.33763 |
| RIKEN cDNA 1810004I06 gene | AV070272 | −4.335500464 | 0.53518 |
| RIKEN cDNA 1810008A14 gene | BG063535 | −8.636021346 | 0.63781 |
| RIKEN cDNA 1810011O01 gene | AV070830 | −5.421078504 | 0.43645 |
| RIKEN cDNA 1810013D10 gene | BG067851 | −4.892379863 | 0.54634 |
| RIKEN cDNA 1810013K23 gene | AW539206 | −4.282626641 | 0.50783 |
| RIKEN cDNA 1810017G16 gene | AV087873 | −7.888058385 | 0.46376 |
| RIKEN cDNA 1810017G16 gene | AV051238 | −4.521324967 | 0.51059 |
| RIKEN cDNA 1810017G16 gene | AV070773 | −4.128355653 | 0.68677 |
| RIKEN cDNA 1810018M11 gene | AV018921 | −9.416192926 | 0.60647 |
| RIKEN cDNA 1810020E01 gene | AV032033 | −5.136798775 | 0.45741 |
| RIKEN cDNA 1810029B16 gene | BG069652 | −6.038729723 | 0.56189 |
| RIKEN cDNA 1810030E18 gene | AV140504 | −5.27469245 | 0.67706 |
| RIKEN cDNA 1810030E20 gene | BG064141 | −4.932956216 | 0.58007 |
| RIKEN cDNA 1810030E20 gene | BG063825 | −4.229066461 | 0.64290 |
| RIKEN cDNA 1810033A19 gene | AV054886 | −5.043468074 | 0.60235 |
| RIKEN cDNA 1810035L17 gene | BG072596 | −5.548484127 | 0.58195 |
| RIKEN cDNA 1810036J22 gene | AV113916 | −19.44625479 | 0.47866 |
| RIKEN cDNA 1810036J22 gene | AV084361 | −5.973172086 | 0.50101 |
| RIKEN cDNA 1810036J22 gene | AV086261 | −5.281464813 | 0.52027 |
| RIKEN cDNA 1810036J22 gene | BG064173 | −5.173272699 | 0.59456 |
| RIKEN cDNA 1810055D05 gene | AV140588 | −5.31258747 | 0.39893 |
| RIKEN cDNA 1810055D05 gene | AV065469 | −4.676521256 | 0.43368 |
| RIKEN cDNA 1810055D05 gene | AV059067 | −5.706489038 | 0.56482 |
| RIKEN cDNA 2010003O02 gene | BG066308 | −4.636818478 | 0.52627 |
| RIKEN cDNA 2010004E11 gene | AV066070 | −5.293676718 | 0.58290 |
| RIKEN cDNA 2010100O12 gene | BG075840 | −5.184355736 | 0.56372 |
| RIKEN cDNA 2010100O12 gene | AV088623 | −7.043681229 | 0.61838 |
| RIKEN cDNA 2010107E04 gene | BG076108 | −4.676770221 | 0.48870 |
| RIKEN cDNA 2010110I09 gene | BG072417 | −8.047056971 | 0.50518 |
| RIKEN cDNA 2010110M21 gene | AV031008 | −4.152271601 | 0.62642 |
| RIKEN cDNA 2010110M21 gene | AV006309 | −5.174330603 | 0.63652 |
| RIKEN cDNA 2210008F15 gene | AV085342 | −6.760958652 | 0.43695 |
| RIKEN cDNA 2210008F15 gene | AV140597 | −4.976752904 | 0.50033 |
| RIKEN cDNA 2210009K14 gene | AV074534 | −4.244231808 | 0.58997 |
| RIKEN cDNA 2210016H18 gene | AW556974 | −4.695260223 | 0.48019 |
| RIKEN cDNA 2210415M14 gene | AV063132 | −4.15138579 | 0.41701 |
| RIKEN cDNA 2210415M14 gene | AV123133 | −6.866891309 | 0.46633 |
| RIKEN cDNA 2210415M14 gene | BG072853 | −5.89983116 | 0.46756 |
| RIKEN cDNA 2210418G03 gene | AV081301 | −7.382877216 | 0.59853 |
| RIKEN cDNA 2310001N14 gene | AV083256 | −9.471464778 | 0.35457 |
| RIKEN cDNA 2310002J21 gene | BG063238 | −4.177926076 | 0.64768 |
| RIKEN cDNA 2310005O14 gene | AV104008 | −5.644497912 | 0.55170 |
| RIKEN cDNA 2310015J09 gene | AV085812 | −5.079301158 | 0.32950 |
| RIKEN cDNA 2310016E22 gene | AV085956 | −4.508187361 | 0.53050 |
| RIKEN cDNA 2310016M24 gene | AV109219 | −6.174685479 | 0.45223 |
| RIKEN cDNA 2310020D23 gene | AA087197 | −4.989916277 | 0.70975 |
| RIKEN cDNA 2310020H20 gene | BG063177 | −4.162978542 | 0.49609 |
| RIKEN cDNA 2310021J10 gene | AV086427 | −5.249829896 | 0.41447 |
| RIKEN cDNA 2310026J01 gene | AV087038 | −6.224052995 | 0.18088 |
| RIKEN cDNA 2310034L04 gene | AV088072 | −4.857617607 | 0.43830 |
| RIKEN cDNA 2310039H15 gene | AV103530 | −5.762586781 | 0.37401 |
| RIKEN cDNA 2310039H15 gene | AV088685 | −10.65523915 | 0.42365 |
| RIKEN cDNA 2310039H15 gene | AV006258 | −4.770080482 | 0.48698 |
| RIKEN cDNA 2310042M24 gene | AV089703 | −4.957830613 | 0.70818 |
| RIKEN cDNA 2310042N02 gene | AV089174 | −5.227461526 | 0.44265 |
| RIKEN cDNA 2310045A07 gene | AV089574 | −5.794732203 | 0.36180 |
| RIKEN cDNA 2310051E17 gene | AV090635 | −5.386354388 | 0.39477 |
| RIKEN cDNA 2310056B04 gene | BG074855 | −4.928886112 | 0.54397 |
| RIKEN cDNA 2310058J06 gene | AV171032 | −5.566735601 | 0.50412 |
| RIKEN cDNA 2310066N05 gene | AV109445 | −4.136380251 | 0.71050 |
| RIKEN cDNA 2310067L22 gene | AV085162 | −6.065666962 | 0.43059 |
| RIKEN cDNA 2310076O14 gene | AV093026 | −5.288222969 | 0.46965 |
| RIKEN cDNA 2310079P10 gene | BG069582 | −10.79467049 | 0.31277 |
| RIKEN cDNA 2400003N08 gene | BG068322 | −5.831862696 | 0.57334 |
| RIKEN cDNA 2400006N03 gene | AV095106 | −5.022967582 | 0.63521 |
| RIKEN cDNA 2400010D15 gene | BG070770 | −5.425606132 | 0.50504 |
| RIKEN cDNA 2400010D15 gene | AV014412 | −5.422633849 | 0.58352 |
| RIKEN cDNA 2400010G15 gene | AV087844 | −5.241042761 | 0.59067 |
| RIKEN cDNA 2410004H02 gene | AV095143 | −4.661273681 | 0.52258 |
| RIKEN cDNA 2410004H02 gene | BG065078 | −4.425936465 | 0.60061 |
| RIKEN cDNA 2410005O16 gene | AV085399 | −4.304045051 | 0.66223 |
| RIKEN cDNA 2410011G03 gene | BG072634 | −7.102554029 | 0.34324 |
| RIKEN cDNA 2410011G03 gene | AV140158 | −7.412258554 | 0.53256 |
| RIKEN cDNA 2410016F19 gene | BG066198 | −4.153805722 | 0.67772 |
| RIKEN cDNA 2410030A14 gene | AV095185 | −4.882546338 | 0.56335 |
| RIKEN cDNA 2410043G19 gene | AV056739 | −5.579786915 | 0.39668 |
| RIKEN cDNA 2410066K11 gene | BG074815 | −4.189499593 | 0.65618 |
| RIKEN cDNA 2410166I05 gene | BG076161 | −7.746565635 | 0.56369 |
| RIKEN cDNA 2510027N19 gene | BG063257 | −4.424035337 | 0.64005 |
| RIKEN cDNA 2510048K03 gene | AV050186 | −7.214847749 | 0.39540 |
| RIKEN cDNA 2600001N01 gene | BG065115 | −4.622808402 | 0.65666 |
| RIKEN cDNA 2610002K22 gene | AV095125 | −4.222224194 | 0.65841 |
| RIKEN cDNA 2610003B19 gene | AV077867 | −5.392435801 | 0.50676 |
| RIKEN cDNA 2610020H15 gene | BG067911 | −4.33184907 | 0.50925 |
| RIKEN cDNA 2610028H24 gene | AU041304 | −8.837908474 | 0.42891 |
| RIKEN cDNA 2610034N03 gene | AV104092 | −4.334279184 | 0.60381 |
| RIKEN cDNA 2610041P16 gene | BG063943 | −9.171542327 | 0.39169 |
| RIKEN cDNA 2610041P16 gene | AV086193 | −4.437390523 | 0.53171 |
| RIKEN cDNA 2610205H19 gene | AV149977 | −5.075180419 | 0.54297 |
| RIKEN cDNA 2610509H23 gene | BG073333 | −4.529188732 | 0.67762 |
| RIKEN cDNA 2610529I12 gene | AV112870 | −4.147133165 | 0.55866 |
| RIKEN cDNA 2700018N07 gene | AI327124 | −4.29762364 | 0.56436 |
| RIKEN cDNA 2700033I16 gene | AV060239 | −4.362623219 | 0.48215 |
| RIKEN cDNA 2700049M22 gene | AU022477 | −6.242566156 | 0.56361 |
| RIKEN cDNA 2700055K07 gene | AV086940 | −5.809367054 | 0.33093 |
| RIKEN cDNA 2700094L05 gene | BG070651 | −6.743245025 | 0.63558 |
| RIKEN cDNA 2810403A07 gene | BG064481 | −4.939425861 | 0.70126 |
| RIKEN cDNA 2810403L02 gene | AI838447 | −5.476484495 | 0.79272 |
| RIKEN cDNA 2810417D04 gene | AV141701 | −4.439903075 | 0.53864 |
| RIKEN cDNA 2810422J05 gene | BG064518 | −5.097975531 | 0.54326 |
| RIKEN cDNA 2810432N10 gene | BG070211 | −4.811203049 | 0.51703 |
| RIKEN cDNA 2810468K05 gene | BG071137 | −5.342157238 | 0.70066 |
| RIKEN cDNA 2900010I05 gene | AV056021 | −4.774554089 | 0.48993 |
| RIKEN cDNA 2900055D03 gene | AV140126 | −4.271457143 | 0.50891 |
| RIKEN cDNA 3110004H13 gene | BG071859 | −6.046421631 | 0.54200 |
| RIKEN cDNA 3110005M08 gene | AV108251 | −4.206377049 | 0.72772 |
| RIKEN cDNA 3200001M24 gene | AV093570 | −4.129969377 | 0.55745 |
| RIKEN cDNA 3200001M24 gene | BG074430 | −4.354466269 | 0.66040 |
| RIKEN cDNA 3230402N08 gene | AV089737 | −4.465701864 | 0.65941 |
| RIKEN cDNA 3830417M17 gene | BG076225 | −4.421284948 | 0.67375 |
| RIKEN cDNA 4432406C05 gene | AV085137 | −6.099053061 | 0.44504 |
| RIKEN cDNA 4631426G04 gene | BG068677 | −4.625459494 | 0.56033 |
| RIKEN cDNA 4632432J16 gene | AV060454 | −4.617958369 | 0.47517 |
| RIKEN cDNA 4633402N23 gene | AA408693 | −5.506478686 | 0.57523 |
| RIKEN cDNA 4833415N24 gene | AV086029 | −4.306972542 | 0.46627 |
| RIKEN cDNA 4833417L20 gene | BG070225 | −4.161297063 | 0.53534 |
| RIKEN cDNA 4930422J18 gene | BG074133 | −6.542937211 | 0.63785 |
| RIKEN cDNA 4930438D12 gene | AV114186 | −5.788046741 | 0.45307 |
| RIKEN cDNA 4930564D15 gene | AW539497 | −6.195679798 | 0.63818 |
| RIKEN cDNA 4933411H20 gene | AV094491 | −10.13251578 | 0.23760 |
| RIKEN cDNA 4933436C10 gene | AI854103 | −9.22185596 | 0.25555 |
| RIKEN cDNA 4933436C10 gene | AV043801 | −7.145276072 | 0.26851 |
| RIKEN cDNA 5430432N15 gene | AV023999 | −5.168897494 | 0.42754 |
| RIKEN cDNA 5730591C18 gene | AV087450 | −4.292004125 | 0.52004 |
| RIKEN cDNA 5830417I10 gene | BG066100 | −4.264697524 | 0.71856 |
| RIKEN cDNA 5830457J20 gene | AV140522 | −5.873234067 | 0.57518 |
| RIKEN cDNA 5830498C14 gene | AV012853 | −10.64307472 | 0.44318 |
| RIKEN cDNA 5830498C14 gene | BG066452 | −4.63710017 | 0.72557 |
| RIKEN cDNA 6030457N17 gene | AV094720 | −11.17974002 | 0.47794 |
| RIKEN cDNA 6430411K18 gene | AV023331 | −6.558273485 | 0.55220 |
| RIKEN cDNA 6530416A09 gene | BG071475 | −6.13803934 | 0.53936 |
| RIKEN cDNA 6720475J19 gene | BG073712 | −13.95563601 | 0.24131 |
| RIKEN cDNA 6720475J19 gene | BG073481 | −7.39081553 | 0.26541 |
| RIKEN cDNA 9030421L11 gene | BG075528 | −4.628327246 | 0.54551 |
| RIKEN cDNA 9130012G04 gene | BG073930 | −6.693464096 | 0.54126 |
| RIKEN cDNA A930018B01 gene | AV073463 | −4.81629501 | 0.73761 |
| RIKEN cDNA E130105L11 gene | BG075577 | −5.960051773 | 0.51388 |
| ring finger protein 11 | AV084728 | −4.227540819 | 0.54992 |
| ring-box 1 | AV053017 | −5.363684395 | 0.58013 |
| RNA polymerase 1-3 (16 kDa subunit) | AV134053 | −4.479915258 | 0.59561 |
| S100 calcium binding protein A1 | AV003587 | −4.795563356 | 0.51956 |
| sacsin | AV013617 | −4.705249687 | 0.67220 |
| S-adenosylmethionine decarboxylase 1 | BG075459 | −6.575072123 | 0.38803 |
| SEC61, gamma subunit (S. cerevisiae) | AV133876 | −4.885488937 | 0.76946 |
| secretory carrier membrane protein 3 | AV094492 | −4.979251312 | 0.43904 |
| serine/threonine kinase 23 | AA170153 | −4.185610913 | 0.46751 |
| serine/threonine kinase 25 (yeast) | AA146115 | −6.421699669 | 0.54596 |
| serologically defined colon cancer antigen 28 | BG065578 | −12.46409454 | 0.18651 |
| serum response factor | AV014460 | −4.179789629 | 0.60298 |
| signal recognition particle 14 kDa (homologous Alu RNA binding protei [symbol] | AV005775 | −7.122752178 | 0.78602 |
| small inducible cytokine A11 | BE137080 | −4.753939259 | 0.43931 |
| small proline rich-like 7 | AV072477 | −4.143398782 | 0.31871 |
| soggy 1 | AV087775 | −4.59725695 | 0.41376 |
| solute carrier family 1, member 7 | AV006313 | −9.007262827 | 0.54179 |
| solute carrier family 16 (monocarboxylic acid transporters), member 2 | AA199215 | −4.248424723 | 0.57730 |
| solute carrier family 25 (mitochondrial carrier; adenine nucleotide trans [symbol] | AV087780 | −4.501100977 | 0.35837 |
| solute carrier family 25 (mitochondrial carrier; oxoglutarate carrier), me [symbol] | AV094940 | −7.980202556 | 0.45584 |
| solute carrier family 27 (fatty acid transporter), member 2 | AA154831 | −6.128882484 | 0.52385 |
| Son cell proliferation protein | BG071049 | −6.036472623 | 0.57640 |
| sortilin-related receptor, LDLR class A repeats-containing | AA673962 | −4.841253747 | 0.44436 |
| special AT-rich sequence binding protein 1 | BG065579 | −6.042197612 | 0.44733 |
| spermine synthase | AV113836 | −4.915770722 | 0.55802 |
| sphingomyelin phosphodiesterase 2, neutral | BG063429 | −4.588922541 | 0.53816 |
| split hand/foot deleted gene 1 | AV134049 | −4.646755588 | 0.56217 |
| steroid 5 alpha-reductase 2-like | AV084563 | −10.28926678 | 0.46589 |
| sterol carrier protein 2, liver | AA146030 | −5.055773043 | 0.61558 |
| succinate-Coenzyme A ligase, GDP-forming, beta subunit | AV087975 | −4.401153724 | 0.54934 |
| superoxide dismutase 1, soluble | BG074045 | −4.775499706 | 0.57536 |
| suppressor of initiator codon mutations, related sequence 1 (S. cerevis [symbol] | AV042274 | −5.892946224 | 0.47109 |
| surfactant associated protein A | AV024739 | −6.312755463 | 0.44949 |
| synaptobrevin like 1 | AV113528 | −11.35230657 | 0.48532 |
| TAR (HIV) RNA binding protein 2 | BG069749 | −4.479592469 | 0.60506 |
| T-box 5 | AA198841 | −5.929892933 | 0.50092 |
| T-cell receptor beta, variable 13 | AV015100 | −5.567729981 | 0.54115 |
| TGF-beta1-induced anti-apoptotic factor 1 | AV078541 | −5.048008293 | 0.68665 |
| thioredoxin 2 | AA116866 | −4.64110901 | 0.58741 |
| thioredoxin-like (32 kD) | AV070815 | −4.571951113 | 0.54871 |
| thioredoxin-like 2 | AV016790 | −5.561621744 | 0.50942 |
| thyroid hormone receptor interactor 13 | AV094724 | −4.603203665 | 0.52873 |
| tight junction protein 1 | BG073399 | −7.525877699 | 0.67799 |
| tissue inhibitor of metalloproteinase 3 | NM_011595 | −7.557159513 | 0.56285 |
| transcription elongation factor A (Sll), 3 | AI322966 | −4.159841646 | 0.34762 |
| transducer of ERBB2, 2 | BG074926 | −5.987030543 | 0.45199 |
| transforming growth factor beta 1 induced transcript 4 | AV140519 | −4.616859427 | 0.74969 |
| transforming growth factor, beta 1 | AA049522 | −8.01904204 | 0.45450 |
| tubulointerstitial nephritis antigen | AV066552 | −4.635666571 | 0.61805 |
| tumor differentially expressed 1, like | AV083974 | −4.20155329 | 0.64214 |
| tumor necrosis factor (ligand) superfamily, member 10 | U37522 | −7.159468126 | 0.44011 |
| tumor necrosis factor receptor superfamily, member 19 | BG072211 | −4.140657689 | 0.34852 |
| tumor necrosis factor, alpha-induced protein 3 | AA572306 | −4.133144105 | 0.60638 |
| ubiquitin-conjugating enzyme E2B, RAD6 homology (S. cerevisiae) | AV095421 | −4.659707734 | 0.55089 |
| ubiquitin-like 3 | BG072313 | −4.13814274 | 0.55812 |
| Unsequenced EST | 413125 | −8.22561445 | 0.22295 |
| Unsequenced EST | 412659 | −8.870617869 | 0.24426 |
| Unsequenced EST | 432064 | −10.13653121 | 0.26718 |
| Unsequenced EST | 410956 | −4.818374482 | 0.26969 |
| Unsequenced EST | 410595 | −5.430746949 | 0.29232 |
| Unsequenced EST | 431252 | −5.030312199 | 0.29553 |
| Unsequenced EST | 411369 | −8.60777606 | 0.29715 |
| Unsequenced EST | 413333 | −4.28197017 | 0.32070 |
| Unsequenced EST | 413297 | −6.333308867 | 0.33170 |
| Unsequenced EST | 411987 | −4.70742313 | 0.33375 |
| Unsequenced EST | 411660 | −8.229104928 | 0.33965 |
| Unsequenced EST | 411054 | −5.207650574 | 0.34062 |
| Unsequenced EST | 410682 | −5.274633509 | 0.34330 |
| Unsequenced EST | 431081 | −5.546409705 | 0.34658 |
| Unsequenced EST | 206294 | −4.181652187 | 0.35033 |
| Unsequenced EST | 412975 | −5.605640895 | 0.35576 |
| Unsequenced EST | 432689 | −5.97281453 | 0.35787 |
| Unsequenced EST | 411277 | −11.08897728 | 0.35956 |
| Unsequenced EST | 412922 | −10.70236842 | 0.36608 |
| Unsequenced EST | 431286 | −4.773151093 | 0.36615 |
| Unsequenced EST | 410681 | −5.539678826 | 0.36806 |
| Unsequenced EST | 410961 | −5.922086756 | 0.36889 |
| Unsequenced EST | 412082 | −5.502268659 | 0.37358 |
| Unsequenced EST | 411260 | −7.318521913 | 0.37963 |
| Unsequenced EST | 413169 | −8.824803866 | 0.38149 |
| Unsequenced EST | 431574 | −7.915188019 | 0.38774 |
| Unsequenced EST | 201627 | −4.705533576 | 0.39533 |
| Unsequenced EST | 411524 | −5.524062307 | 0.39648 |
| Unsequenced EST | 207603 | −4.355050407 | 0.39946 |
| Unsequenced EST | 411380 | −7.305463236 | 0.40609 |
| Unsequenced EST | 412118 | −5.556347655 | 0.40838 |
| Unsequenced EST | 412779 | −5.441554043 | 0.40976 |
| Unsequenced EST | 413183 | −4.193228901 | 0.41145 |
| Unsequenced EST | 412186 | −5.014710177 | 0.41232 |
| Unsequenced EST | 412432 | −6.021307948 | 0.41525 |
| Unsequenced EST | 202131 | −4.528895291 | 0.42149 |
| Unsequenced EST | 411977 | −5.552286122 | 0.42892 |
| Unsequenced EST | 411945 | −5.19632995 | 0.43045 |
| Unsequenced EST | 412392 | −5.259013295 | 0.43294 |
| Unsequenced EST | 411789 | −5.942433491 | 0.43374 |
| Unsequenced EST | 411605 | −4.341117607 | 0.43784 |
| Unsequenced EST | 412744 | −7.339592203 | 0.43951 |
| Unsequenced EST | 413539 | −4.989934344 | 0.44370 |
| Unsequenced EST | 195728 | −6.178492322 | 0.44536 |
| Unsequenced EST | 413134 | −6.241885103 | 0.45027 |
| Unsequenced EST | 411383 | −5.401353982 | 0.45800 |
| Unsequenced EST | 411085 | −4.137943214 | 0.46202 |
| Unsequenced EST | 412790 | −4.941794716 | 0.46286 |
| Unsequenced EST | 412128 | −4.173237872 | 0.46629 |
| Unsequenced EST | 412515 | −4.302837338 | 0.47046 |
| Unsequenced EST | 411160 | −4.39905373 | 0.47073 |
| Unsequenced EST | 431843 | −4.915899211 | 0.47188 |
| Unsequenced EST | 412684 | −4.241205638 | 0.47318 |
| Unsequenced EST | 412861 | −8.341188453 | 0.47330 |
| Unsequenced EST | 412655 | −7.654529341 | 0.47341 |
| Unsequenced EST | 412947 | −5.987474705 | 0.47730 |
| Unsequenced EST | 431845 | −6.589036532 | 0.47756 |
| Unsequenced EST | 412605 | −4.545499757 | 0.47830 |
| Unsequenced EST | 412852 | −5.666295082 | 0.48040 |
| Unsequenced EST | 412719 | −6.436286215 | 0.48313 |
| Unsequenced EST | 412846 | −6.379601248 | 0.48331 |
| Unsequenced EST | 411516 | −4.186279748 | 0.48381 |
| Unsequenced EST | 430640 | −8.543745358 | 0.48480 |
| Unsequenced EST | 413600 | −4.901398844 | 0.48861 |
| Unsequenced EST | 410665 | −5.244586119 | 0.48898 |
| Unsequenced EST | 412580 | −4.121077374 | 0.49239 |
| Unsequenced EST | 412961 | −6.883843851 | 0.49284 |
| Unsequenced EST | 410750 | −4.49336413 | 0.49891 |
| Unsequenced EST | 413575 | −8.092713979 | 0.49917 |
| Unsequenced EST | 412258 | −4.851281671 | 0.50038 |
| Unsequenced EST | 413527 | −5.132468462 | 0.50202 |
| Unsequenced EST | 339227 | −5.039795897 | 0.50472 |
| Unsequenced EST | 412794 | −4.990410609 | 0.50493 |
| Unsequenced EST | 413170 | −4.535280662 | 0.50708 |
| Unsequenced EST | 412554 | −5.450841531 | 0.51085 |
| Unsequenced EST | 411061 | −4.769542333 | 0.51494 |
| Unsequenced EST | 413191 | −4.260493159 | 0.51664 |
| Unsequenced EST | 411529 | −4.146671502 | 0.51863 |
| Unsequenced EST | 201438 | −5.686498384 | 0.51877 |
| Unsequenced EST | 412188 | −5.828768851 | 0.53010 |
| Unsequenced EST | 412687 | −4.271665088 | 0.53249 |
| Unsequenced EST | 411735 | −4.468462406 | 0.53596 |
| Unsequenced EST | 432195 | −4.335845288 | 0.53607 |
| Unsequenced EST | 431862 | −6.165660675 | 0.54297 |
| Unsequenced EST | 431724 | −4.338553681 | 0.54756 |
| Unsequenced EST | 202908 | −5.418394672 | 0.54969 |
| Unsequenced EST | 413323 | −4.184245611 | 0.55110 |
| Unsequenced EST | 411704 | −5.096046224 | 0.55200 |
| Unsequenced EST | 412581 | −5.269737426 | 0.55208 |
| Unsequenced EST | 412585 | −4.659918123 | 0.55273 |
| Unsequenced EST | 431810 | −4.180748837 | 0.55450 |
| Unsequenced EST | 413365 | −4.2659871 | 0.55605 |
| Unsequenced EST | 433229 | −4.517254893 | 0.56214 |
| Unsequenced EST | 411979 | −4.346159953 | 0.56235 |
| Unsequenced EST | 413165 | −4.62951073 | 0.56443 |
| Unsequenced EST | 192693 | −5.043346885 | 0.56552 |
| Unsequenced EST | 431411 | −4.213334563 | 0.56581 |
| Unsequenced EST | 413343 | −4.858667556 | 0.56811 |
| Unsequenced EST | 431024 | −4.530557713 | 0.57100 |
| Unsequenced EST | 411004 | −5.585263324 | 0.57150 |
| Unsequenced EST | 412778 | −4.958457315 | 0.57369 |
| Unsequenced EST | 411679 | −4.397694818 | 0.57591 |
| Unsequenced EST | 412092 | −4.601171247 | 0.57736 |
| Unsequenced EST | 411187 | −5.420404234 | 0.57748 |
| Unsequenced EST | 412049 | −4.182454971 | 0.57918 |
| Unsequenced EST | 411739 | −5.261687986 | 0.57938 |
| Unsequenced EST | 412792 | −5.800493052 | 0.58184 |
| Unsequenced EST | 430792 | −4.281087478 | 0.58252 |
| Unsequenced EST | 412248 | −6.65590185 | 0.58382 |
| Unsequenced EST | 411820 | −5.940618083 | 0.58997 |
| Unsequenced EST | 412944 | −5.470273005 | 0.59317 |
| Unsequenced EST | 413551 | −4.582248971 | 0.59406 |
| Unsequenced EST | 411432 | −20.53697874 | 0.59957 |
| Unsequenced EST | 410575 | −5.303084684 | 0.60532 |
| Unsequenced EST | 412300 | −4.818706528 | 0.61404 |
| Unsequenced EST | 413127 | −4.268879629 | 0.61420 |
| Unsequenced EST | 413147 | −4.834386905 | 0.61435 |
| Unsequenced EST | 431502 | −4.610470753 | 0.61626 |
| Unsequenced EST | 412669 | −6.722369522 | 0.62754 |
| Unsequenced EST | 205043 | −4.492534174 | 0.62848 |
| Unsequenced EST | 411951 | −4.241151187 | 0.63106 |
| Unsequenced EST | 410855 | −7.411266903 | 0.63325 |
| Unsequenced EST | 431873 | −4.381828532 | 0.64516 |
| Unsequenced EST | 413577 | −4.117483105 | 0.64824 |
| Unsequenced EST | 412322 | −5.050800613 | 0.65809 |
| Unsequenced EST | 431604 | −4.652721214 | 0.65891 |
| Unsequenced EST | 410853 | −5.906498521 | 0.67231 |
| Unsequenced EST | 410873 | −5.013976686 | 0.68258 |
| Unsequenced EST | 411493 | −5.338523882 | 0.68321 |
| Unsequenced EST | 411809 | −4.799364595 | 0.70861 |
| Unsequenced EST | 431869 | −5.019525302 | 0.70973 |
| Unsequenced EST | 410832 | −4.976967369 | 0.72665 |
| Unsequenced EST | 413270 | −4.343167788 | 0.75177 |
| upregulated during skeletal muscle growth 5 | AV088589 | −4.446982985 | 0.45597 |
| vesicle-associated membrane protein 2 | AW911135 | −4.74028883 | 0.67738 |
| vesicle-associated membrane protein 3 | AV085364 | −4.433657569 | 0.34943 |
| voltage-dependent anion channel 1 | BG073650 | −4.530236983 | 0.55543 |
| wingless-related MMTV integration site 3A | AA000971 | −5.545510401 | 0.58208 |
| Y box protein 2 | BG066570 | −4.568246796 | 0.43028 |
| Yamaguchi sarcoma viral (v-yes-1) oncogene homolog | AA509398 | −4.224596131 | 0.55530 |
| zinc finger protein 106 | AV013127 | −4.399813491 | 0.43000 |
| zinc finger protein 216 | BG066068 | −17.41108393 | 0.55649 |

TABLE IA

| Gene Name | Gene Description | UGRepAcc [A] | LLRepProtAc [symbol] |
|---|---|---|---|
| AA068104 | transforming growth factor, beta 2 | NM_009367 | NP_033393 |
| AA098349 | lysyl oxidase-like | AK078512 | |
| AA498724 | bone morphogenetic protein 4 | NM_007554 | NP_031580 |
| AA646363 | endoglin | NM_007932 | NP_031958 |
| AI323974 | neuropilin | NM_008737 | NP_032763 |
| AI327133 | polydomain protein | NM_022814 | NP_073725 |
| AI841353 | a disintegrin and metalloproteinase domain 15 (metar | NM_009614 | NP_033744 |
| AV012617 | insulin-like growth factor binding protein 5 | NM_010518 | NP_034648 |
| AV015188 | matrix metalloproteinase 23 | NM_011985 | NP_036115 |
| AV019210 | elastin | NM_007925 | NP_031951 |
| AV021712 | secreted frizzled-related sequence protein 2 | NM_009144 | NP_033170 |
| AV024396 | reversion-inducing-cysteine-rich protein with kazal m [symbol] | NM_016678 | NP_057887 |
| AV029310 | superoxide dismutase 3, extracellular | NM_011435 | NP_035565 |
| AV059520 | peptidylprolyl isomerase C-associated protein | NM_011150 | NP_035280 |
| AV070218 | amyloid beta (A4) precursor-like protein 2 | NM_009691 | NP_033821 |
| AV070419 | antigen identified by monoclonal antibody MRC OX-2 | NM_010818 | NP_034948 |
| AV083867 | retinoid-inducible serine caroboxypetidase | NM_029023 | NP_083299 |
| AV084876 | osteoblast specific factor 2 (fasciclin I-like) | NM_015784 | NP_056599 |
| AV085019 | extracellular matrix protein 1 | NM_007899 | NP_031925 |
| AV104097 | basigin | BI106083 | |
| AV104213 | endothelial cell-selective adhesion molecule | NM_027102 | NP_081378 |
| AV109513 | stromal cell derived factor 1 | NM_013655 | NP_068350 |
| AV113097 | microfibrillar associated protein 5 | NM_015776 | NP_056591 |
| AV117035 | manic fringe homolog (Drosophila) | NM_008595 | NP_032621 |
| AV149987 | cystatin C | NM_009976 | NP_034106 |
| AV156534 | matrilin 2 | NM_016762 | NP_058042 |
| AV170826 | biglycan | NM_007542 | NP_031568 |
| AW476537 | fibroblast growth factor receptor 1 | NM_010206 | NP_034336 |
| AW988741_{—} | secreted acidic cysteine rich glycoprotein | | |
| BE376968 | vascular endothelial growth factor C | NM_009506 | NP_033532 |
| BF136770 | Notch gene homolog 3, (Drosophila) | NM_008716 | NP_032742 |
| BG063294 | follistatin-like 3 | NM_031380 | NP_113557 |
| BG063616 | nidogen 1 | NM_010917 | NP_035047 |
| BG064180 | expressed sequence AA408225 | NM_009868 | NP_033998 |
| BG065640 | ectonucleotide pyrophosphatase/phosphodiesterase | NM_008813 | NP_032839 |
| BG066563 | N-acetylated alpha-linked acidic dipeptidase 2 | NM_028279 | NP_082555 |
| BG073227 | fibulin 2 | NM_007992 | NP_032018 |
| BG074344 | mesothelin | NM_018857 | NP_061345 |
| BG074382 | sema domain, immunoglobulin domain (Ig), short bas [symbol] | NM_011349 | NP_035479 |
| BG074663 | protein tyrosine phosphatase, receptor type, S | NM_011218 | NP_035348 |
| BG075377 | melanoma cell adhesion molecule | NM_023061 | NP_075548 |
| D16250 | bone morphogenetic protein receptor, type 1A | BC042611 | NP_033888 |
| L26349 | tumor necrosis factor receptor superfamily, member 1 | NM_011609 | NP_035739 |
| U38261 | superoxide dismutase 3, extracellular | NM_011435 | NP_035565 |
| X52886 | cathepsin D | NM_009983 | NP_034113 |
| AI838311 | matrix metalloproteinase 2 | NM_008610 | NP_032636 |
| AI851067 | RIKEN cDNA 2510010F10 gene | NM_175833 | NP_787027 |
| BG071948 | low density lipoprotein receptor-related protein 1 | NM_008512 | NP_032538 |
| BG072998 | expressed sequence AU018638 | NM_008524 | NP_032550 |
| AI838613 | epithelial membrane protein 1 | | |
| AI893233 | CD34 antigen | NM_133654 | NP_598415 |
| AV001464 | granulin | NM_008175 | NP_032201 |
| AV006514 | interferon (alpha and beta) receptor 2 | NM_010509 | NP_034639 |
| AV022379 | serine (or cysteine) proteinase inhibitor, clade F (alph [symbol] | NM_011340 | NP_035470 |
| AV025941 | aquaporin 1 | NM_007472 | NP_031498 |
| AV070805 | thymic stromal-derived lymphopoietin, receptor | NM_016715 | NP_057924 |
| AV223941 | heat shock protein, 70 kDa 3 | M12571 | |
| AW537378 | EST | | |
| AA673390 | fibronectin 1 | AK090130 | |
| AI325851 | CD97 antigen | NM_011925 | NP_036055 |
| AI325886 | neuroblastoma, suppression of tumorigenicity 1 | NM_008675 | NP_032701 |
| AI385650 | sialyltransferase 4C (beta-galactosidase alpha-2,3-si [symbol] | NM_009178 | NP_033204 |
| AI838302 | Cd63 antigen | NM_007653 | NP_031679 |
| AI838568 | RIKEN cDNA 1300018J16 gene | NM_029092 | NP_083368 |
| AV007183 | latent transforming growth factor beta binding protein | NM_023912 | NP_076401 |
| AV007276 | RIKEN cDNA 1110003M08 gene | AK090329 | |
| AV009300 | procollagen, type IV, alpha 1 | J04694 | |
| AV010312 | procollagen, type IV, alpha 2 | J04695 | |
| AV011166 | EST | NM_080463 | NP_536711 |
| AV013988 | procollagen, type VI, alpha 1 | NM_009933 | NP_034063 |
| AV015595 | procollagen, type XV | NM_009928 | NP_034058 |
| AV016743 | RIKEN cDNA 5730414C17 gene | NM_133680 | NP_598441 |
| AV025665 | prostaglandin-endoperoxide synthase 2 | NM_011198 | NP_035328 |
| AV036454_ [symbol] | lymphocyte antigen 6 complex, locus E | | |
| AV037769 | expressed sequence AU022549 | NM_007904 | NP_031930 |
| AV048780 | stromal cell derived factor 4 | NM_011341 | NP_035471 |
| AV050682 | RIKEN cDNA 2700083B06 gene | NM_026531 | NP_080807 |
| AV052090 | serine (or cysteine) proteinase inhibitor, clade I (neur [symbol] | NM_009250 | NP_033276 |
| AV053955 | RIKEN cDNA 3110023E09 gene | NM_026522 | NP_080798 |
| AV057827 | torsin family 3, member A | NM_023141 | NP_075630 |
| AV058250 | RIKEN cDNA 1810049K24 gene | NM_030209 | NP_084485 |
| AV059445 | FK506 binding protein 9 | NM_012056 | NP_036186 |
| AV059924 | expressed sequence AA986889 | NM_134102 | NP_598863 |
| AV061081 | neural proliferation, differentiation and control gene 1 | NM_008721 | NP_032747 |
| AV062071 | CD24a antigen | NM_009846 | NP_033976 |
| AV066211 | ELAV (embryonic lethal, abnormal vision, Drosophila) | NM_010485 | NP_034615 |
| AV073997 | glucose regulated protein, 58 kDa | NM_007952 | NP_031978 |
| AV083352 | RIKEN cDNA 1110007F23 gene | NM_029568 | NP_083844 |
| AV084561 | procollagen C-proteinase enhancer protein | NM_008788 | NP_032814 |
| AV084844 | immunoglobulin superfamily containing leucine-rich r [symbol] | NM_012043 | NP_036173 |
| AV086002 | FXYD domain-containing ion transport regulator 6 | NM_022004 | NP_071287 |
| AV087039 | EST | NM_008885 | NP_032911 |
| AV087220 | expressed sequence AW146116 | NM_133352 | NP_835359 |
| AV087499 | EST, Moderately similar to A57474 extracellular matri | NM_007899 | NP_031925 |
| AV087921 | benzodiazepine receptor, peripheral | NM_009775 | NP_033905 |
| AV089105 | calcium binding protein, intestinal | NM_009787 | NP_033917 |
| AV093463 | serine (or cysteine) proteinase inhibitor, clade H (hea [symbol] | NM_009825 | NP_033955 |
| AV094498 | milk fat globule-EGF factor 8 protein | NM_008594 | NP_032620 |
| AV103290 | expressed sequence AL024047 | NM_134151 | NP_598912 |
| AV104157 | dolichyl-di-phosphooligosaccharide-protein glycotrans | NM_007838 | NP_031864 |
| AV109555 | cellular retinoic acid binding protein I | AK090130 | |
| AV111526 | RIKEN cDNA 2610002H11 gene | NM_133721 | NP_598482 |
| AV112983 | platelet derived growth factor receptor, beta polypepti [symbol] | NM_008809 | NP_032835 |
| AV133755 | RIKEN cDNA 2810002E22 gene | NM_133859 | NP_598620 |
| AV134035 | granulin | NM_008175 | NP_032201 |
| AV140189 | RIKEN cDNA 0610040B21 gene | NM_025334 | NP_079610 |
| AV140901 | EST | NM_010368 | NP_034498 |
| AV162270 | lymphocyte antigen 6 complex, locus A | NM_027015 | NP_081291 |
| AV171867 | CD 81 antigen | NM_133655 | NP_598416 |
| AW548258 | procollagen-proline, 2-oxoglutarate 4-dioxygenase (p [symbol] | BC009654 | |
| AW551778 | heterogeneous nuclear ribonucleoprotein C | NM_016884 | NP_058580 |
| BF100414 | integrin beta 5 | NM_010580 | NP_034710 |
| BF182158 | Notch gene homolog 1, (Drosophila) | NM_008714 | NP_032740 |
| BG063167 | adenylate cyclase 7 | NM_007406 | NP_031432 |
| BG065103 | lymphocyte antigen 6 complex, locus E | NM_008529 | NP_032555 |
| BG066621 | Mus musculus, Similar to pituitary tumor-transforming | NM_145925 | NP_666037 |
| BG067569 | coagulation factor II (thrombin) receptor | NM_010169 | NP_034299 |
| BG069745 | proline arginine-rich end leucine-rich repeat | NM_054077 | NP_473418 |
| BG070083 | protein tyrosine phosphatase, receptor type, E | NM_011212 | NP_035342 |
| BG070387 | interleukin 6 signal transducer | NM_010560 | NP_034690 |
| BG072624 | laminin, gamma 1 | BC032194 | NP_034813 |
| BG072810 | Niemann Pick type 02 | NM_023409 | NP_075898 |
| BG072850 | sarcoglycan, epsilon | NM_011360 | NP_035490 |
| BG072908 | membrane-bound transcription factor protease, site 1 | NM_019709 | NP_062683 |
| BG073140 | CD8 antigen, beta chain | NM_009858 | NP_033988 |
| BG073341 | retinal short-chain dehydrogenase/reductase 1 | NM_011303 | NP_035433 |
| BG073479 | expressed sequence AW229038 | NM_133918 | NP_598679 |
| BG073729 | prolyl 4-hydroxylase, beta polypeptide | J05185 | |
| BG073750 | prolyl 4-hydroxylase, beta polypeptide | J05185 | |
| BG074142 | RIKEN cDNA 1300012G16 gene | NM_023625 | NP_076114 |
| BG074174 | DNA segment, Chr 6, Wayne State University 176 e [symbol] | NM_138587 | NP_613053 |
| BG074422 | integrin beta 1 (fibronectin receptor beta) | AK088016 | |
| BG074747 | alpha glucosidase 2, alpha neutral subunit | NM_008060 | NP_032086 |
| BG074915 | parotid secretory protein | NM_172261 | NP_758465 |
| BG075864 | procollagen, type VI, alpha 2 | NM_146007 | NP_666119 |
| C79946 | expressed sequence C79946 | AK080023 | |
| U20156 | EST | | |
| U34920 | ATP-binding cassette, sub-family G (WHITE), membe [symbol] | NM_009593 | NP_033723 |
| X00246 | histocompatibility 2, D region locus 1 | NM_010380 | NP_034510 |
| X01838 | beta-2 microglobulin | NM_009735 | NP_033865 |
| AA087526 | retinol binding protein 1, cellular | NM_011254 | NP_035384 |
| AI322274 | RIKEN cDNA 2410002J21 gene | AK033091 | |
| AI851039 | ESTs, Weakly similar to D2045.2.p [Caenorhabditis e [symbol] | AK038775 | |
| AV015246 | RIKEN cDNA 1110054M18 gene | NM_175132 | NP_780341 |
| AV057141 | gap junction membrane channel protein beta 1 | NM_008124 | NP_032150 |
| AV059438 | ets variant gene 6 (TEL oncogene) | BC009120 | |
| AV077899 | actin, alpha 2, smooth muscle, aorta | AK002886 | |
| AV083262 | dystonin | NM_134448 | NP_604443 |
| AV083596 | four and a half LIM domains 1 | NM_010211 | NP_034341 |
| AV085874 | Mus musculus uridindiphosphoglucosepyrophosphor [symbol] | NM_139297 | NP_647458 |
| AV093704 | small EDRK-rich factor 2 | AK044479 | |
| AW547864 | EST | | |
| BG065584 | Mus musculus, clone IMAGE: 3589087, mRNA, partia [symbol] | BF124761 | |
| BG070007 | expressed sequence AW494241 | BC040467 | |
| BG072752 | actin, gamma, cytoplasmic | NM_013798 | NP_038826 |
| BG073284 | prion protein dublet | NM_023043 | NP_075530 |
| BG073319 | integrin beta 4 binding protein | NM_010579 | NP_034709 |

TABLE IB

| Gene Name | Gene Description | UGRepAcc [A] | LLRepProtAcc [A] |
|---|---|---|---|
| AA068104 | transforming growth factor, beta 2 | NM_009367 | NP_033393 |
| AA098349 | lysyl oxidase-like | AK078512 | |
| AA498724 | bone morphogenetic protein 4 | NM_007554 | NP_031580 |
| AA646363 | endoglin | NM_007932 | NP_031958 |
| AI323974 | neuropilin | NM_008737 | NP_032763 |
| AI327133 | polydomain protein | NM_022814 | NP_073725 |
| AI841353 | a disintegrin and metalloproteinase domain 15 (met [symbol] | NM_009614 | NP_033744 |
| AV012617 | insulin-like growth factor binding protein 5 | NM_010518 | NP_034648 |
| AV015188 | matrix metalloproteinase 23 | NM_011985 | NP_036115 |
| AV019210 | elastin | NM_007925 | NP_031951 |
| AV021712 | secreted frizzled-related sequence protein 2 | NM_009144 | NP_033170 |
| AV024396 | reversion-inducing-cysteine-rich protein with kazal n [symbol] | NM_016678 | NP_057887 |
| AV029310 | superoxide dismutase 3, extracellular | NM_011435 | NP_035565 |
| AV059520 | peptidylprolyl isomerase C-associated protein | NM_011150 | NP_035280 |
| AV070218 | amyloid beta (A4) precursor-like protein 2 | NM_009691 | NP_033821 |
| AV070419 | antigen identified by monoclonal antibody MRC OX- [symbol] | NM_010818 | NP_034948 |
| AV083867 | retinoid-inducible serine caroboxypetidase | NM_029023 | NP_083299 |
| AV084876 | osteoblast specific factor 2 (fasciclin I-like) | NM_015784 | NP_056599 |
| AV085019 | extracellular matrix protein 1 | NM_007899 | NP_031925 |
| AV104097 | basigin | BI106083 | |
| AV104213 | endothelial cell-selective adhesion molecule | NM_027102 | NP_081378 |
| AV109513 | stromal cell derived factor 1 | NM_013655 | NP_068350 |
| AV113097 | microfibrillar associated protein 5 | NM_015776 | NP_056591 |
| AV117035 | manic fringe homolog (Drosophila) | NM_008595 | NP_032621 |
| AV149987 | cystatin C | NM_009976 | NP_034106 |
| AV156534 | matrilin 2 | NM_016762 | NP_058042 |
| AV170826 | biglycan | NM_007542 | NP_031568 |
| AW476537 | fibroblast growth factor receptor 1 | NM_010206 | NP_034336 |
| AW988741_{——}2 | secreted acidic cysteine rich glycoprotein | | |
| BE376968 | vascular endothelial growth factor C | NM_009506 | NP_033532 |
| BF136770 | Notch gene homolog 3, (Drosophila) | NM_008716 | NP_032742 |
| BG063294 | follistatin-like 3 | NM_031380 | NP_113557 |
| BG063616 | nidogen 1 | NM_010917 | NP_035047 |
| BG064180 | expressed sequence AA408225 | NM_009868 | NP_033998 |
| BG065640 | ectonucleotide pyrophosphatase/phosphodiesterase | NM_008813 | NP_032839 |
| BG066563 | N-acetylated alpha-linked acidic dipeptidase 2 | NM_028279 | NP_082555 |
| BG073227 | fibulin 2 | NM_007992 | NP_032018 |
| BG074344 | mesothelin | NM_018857 | NP_061345 |
| BG074382 | sema domain, immunoglobulin domain (Ig), short b [symbol] | NM_011349 | NP_035479 |
| BG074663 | protein tyrosine phosphatase, receptor type, S | NM_011218 | NP_035348 |
| BG075377 | melanoma cell adhesion molecule | NM_023061 | NP_075548 |
| D16250 | bone morphogenetic protein receptor, type 1A | BC042611 | NP_033888 |
| L26349 | tumor necrosis factor receptor superfamily, member [symbol] | NM_011609 | NP_035739 |
| U38261 | superoxide dismutase 3, extracellular | NM_011435 | NP_035565 |
| X52886 | cathepsin D | NM_009983 | NP_034113 |
| AI838311 | matrix metalloproteinase 2 | NM_008610 | NP_032636 |
| AI851067 | RIKEN cDNA 2510010F10 gene | NM_175833 | NP_787027 |
| BG071948 | low density lipoprotein receptor-related protein 1 | NM_008512 | NP_032538 |
| BG072998 | expressed sequence AU018638 | NM_008524 | NP_032550 |
| AI838613 | epithelial membrane protein 1 | | |
| AI893233 | CD34 antigen | NM_133654 | NP_598415 |
| AV001464 | granulin | NM_008175 | NP_032201 |
| AV006514 | interferon (alpha and beta) receptor 2 | NM_010509 | NP_034639 |
| AV022379 | serine (or cysteine) proteinase inhibitor, clade F (al [symbol] | NM_011340 | NP_035470 |
| AV025941 | aquaporin 1 | NM_007472 | NP_031498 |
| AV070805 | thymic stromal-derived lymphopoietin, receptor | NM_016715 | NP_057924 |

TABLE II
Table II Genes of Use in Imaging Studies - Membrane Associated
Annotated Extracellular and Antigen genes Upregulated in TAC tissues - 149 Unique genes
One example for each gene - Passed stringent SAM criteria

| Mouse Gene Information | | | | | | Human Homolog Information | |
|---|---|---|---|---|---|---|---|
| Gene ID | Gene Description | UGRepAcc | LLRepProtAcc | Up TAC LA | Up TAC LV | UGRepAcc | LLRepProtAcc |
| BG073140 | **CD8 antigen, beta chain | NM_009858 | NP_033988 | UP TAC LA | | | |
| AI841353 | a disintegrin and metalloproteinase domain | NM_009614 | NP_033744 | UP TAC LA | | AY560601 | NP_997080 |
| | 15 (metargidin) | | | | | | |
| AV024684 | A kinase (PRKA) anchor protein 2 | NM_009649 | NP_033779 | UP TAC LA | | | |
| AA797434 | adenylate cyclase 7 | NM_007406 | NP_031432 | UP TAC LA | | D25538 | NP_001105 |
| AV103043 | ADP-ribosylation factor 4 | NM_007479 | NP_031505 | UP TAC LA | | BC016325 | NP_001651 |
| AV032992 | ADP-ribosylation-like factor 6 interacting | NM_022992 | NP_075368 | UP TAC LA | | | |
| | protein 5 | | | | | | |
| AV057752 | amyloid beta (A4) precursor protein | NM_007471 | NP_031497 | UP TAC LA | | BC018937 | NP_958817 |
| AV104479 | amyloid beta (A4) precursor protein-binding, | AK004792 | | UP TAC LA | | | |
| | family B, member 2 | | | | | | |
| AV070218 | amyloid beta (A4) precursor-like protein 2 | NM_009691 | NP_033821 | UP TAC LA | | BX647107 | NP_001633 |
| AV043404 | angiotensin converting enzyme | | | UP TAC LA | | | |
| AV025146 | angiotensin receptor-like 1 | NM_011784 | NP_035914 | UP TAC LA | | AK075252 | NP_005152 |
| AV163403 | antigen identified by monoclonal antibody | NM_010818 | NP_034948 | UP TAC LA | | BC022522 | NP_005935 |
| | MRC OX-2 | | | | | | |
| AV025941 | aquaporin 1 | NM_007472 | NP_031498 | UP TAC LA | | NM_198098 | NP_932766 |
| AV173744 | ATPase, Cu++ transporting, alpha | NM_009726 | NP_033856 | UP TAC LA | | NM_000052 | NP_000043 |
| | polypeptide | | | | | | |
| AV031502 | ATPase, H+ transporting, lysosomal 70 kD, | BI100125 | | UP TAC LA | | AK023063 | NP_006326 |
| | V1 subunit A, isoform 1 [symbol] | | | | | | |
| U34920 | ATP-binding cassette, sub-family G | NM_009593 | NP_033723 | UP TAC LA | | NM_207630 | NP_997513 |
| | (WHITE), member 1 | | | | | | |
| BG064525 | basigin | BI106083 | | UP TAC LA | | NM_001728 | NP_940993 |
| AV104535 | beclin 1 (coiled-coil, myosin-like | NM_026562 | NP_080838 | UP TAC LA | | | |
| | BCL2-interacting protein) | | | | | | |
| AV087921 | benzodiazepine receptor, peripheral | NM_009775 | NP_033905 | UP TAC LA | | BX537892 | NP_009295 |
| X01838 | beta-2 microglobulin | NM_009735 | NP_033865 | UP TAC LA | | AK022379 | NP_004039 |
| AV140458 | biregional cell adhesion molecule-related/ | NM_172506 | NP_766094 | UP TAC LA | | NM_033254 | NP_150279 |
| | down-regulated by oncog [symbol] | | | | | | |
| D16250 | bone morphogenetic protein receptor, | BC042611 | NP_033888 | UP TAC LA | | NM_004329 | NP_004320 |
| | type 1A | | | | | | |
| BG065470 | catenin beta | NM_177589 | NP_808257 | UP TAC LA | | | |
| AV171867 | CD 81 antigen | NM_133655 | NP_598416 | UP TAC LA | | BM810055 | NP_004347 |
| AV062071 | CD24a antigen | NM_009846 | NP_033976 | UP TAC LA | | | |
| AI893233 | CD34 antigen | NM_133654 | NP_598415 | UP TAC LA | | BX640941 | NP_001764 |
| BG073167 | Cd63 antigen | NM_007653 | NP_031679 | UP TAC LA | | BM701371 | NP_001771 |
| AI325851 | CD97 antigen | NM_011925 | NP_036055 | UP TAC LA | | NM_078481 | NP_510966 |
| AV300841 | chemokine (C—X—C) receptor 4 | | | UP TAC LA | | NM_003467 | NP_003458 |
| BG067569 | coagulation factor II (thrombin) receptor | NM_010169 | NP_034299 | UP TAC LA | | NM_001992 | NP_001983 |
| AV031224 | coatomer protein complex, subunit gamma 1 | NM_017477 | NP_059505 | UP TAC LA | | | |
| AV147446 | cytochrome P450, 2j6 | | | UP TAC LA | | | |
| AV037185 | degenerative spermatocyte homolog | NM_007853 | NP_031879 | UP TAC LA | | NM_003676 | NP_659004 |
| | (Drosophila) | | | | | | |
| AV083741 | DNA segment, Chr 8, Brigham & Women's | NM_026002 | NP_080278 | UP TAC LA | | | |
| | Genetics 1112 express [symbol] | | | | | | |
| AV104157 | dolichyl-di-phosphooligosaccharide-protein | NM_007838 | NP_031864 | UP TAC LA | | NM_005216 | NP_005207 |
| | glycotransferase | | | | | | |
| BG075775 | downstream of tyrosine kinase 1 | NM_010070 | NP_034200 | UP TAC LA | | AK055944 | NP_001372 |
| BG065640 | ectonucleotide pyrophosphatase/ | NM_008813 | NP_032839 | UP TAC LA | | NM_006208 | NP_006199 |
| | phosphodiesterase 1 | | | | | | |
| AV050518 | elongation of very long chain fatty acids | NM_019422 | NP_062295 | UP TAC LA | | NM_022821 | NP_073732 |
| | (FEN1/Elo2, SUR4/Elo3, y [symbol] | | | | | | |
| AV140302 | embigin | NM_010330 | NP_034460 | UP TAC LA | | | |
| AV086531 | endoglin | NM_007932 | NP_031958 | UP TAC LA | | NM_000118 | NP_000109 |
| AV104213 | endothelial cell-selective adhesion molecule | NM_027102 | NP_081378 | UP TAC LA | | | |
| AI838613 | epithelial membrane protein 1 | | | UP TAC LA | UP TAC LV | NM_001423 | NP_001414 |
| AV087039 | EST | NM_008885 | NP_032911 | UP TAC LA | | NM_000304 | NP_696997 |
| AV087918 | EST AA087124 | AA896198 | | UP TAC LA | | NM_001759 | NP_001750 |
| AV021942 | ESTs, Weakly similar to ATPase, class 1, | AF156546 | | UP TAC LA | | AB032963 | NP_065185 |
| | member a; ATPase 8A2 [symbol] | | | | | | |
| AV016534 | ESTs, Weakly similar to Y43F4B.7.p | NM_153170 | NP_694810 | UP TAC LA | | | |
| | [Caenorhabditis elegans] [C.e [symbol] | | | | | | |
| AV113175 | ETL1 | NM_133222 | NP_573485 | UP TAC LA | | AY358360 | |
| BG064180 | expressed sequence AA408225 | NM_009868 | NP_033998 | UP TAC LA | | NM_001795 | NP_001786 |
| BG072659 | expressed sequence AI316797 | NM_080563 | NP_542130 | UP TAC LA | | NM_014746 | NP_055561 |
| AV033704 | expressed sequence AI504145 | NM_028990 | NP_083266 | UP TAC LA | | | |
| AV037769 | expressed sequence AU022549 | NM_007904 | NP_031930 | UP TAC LA | | NM_000115 | NP_003982 |
| AV087220 | expressed sequence AW146116 | NM_133352 | NP_835359 | UP TAC LA | | | |
| BG066820 | expressed sequence C80501 | NM_009320 | NP_033346 | UP TAC LA | | NM_003043 | NP_003034 |
| AW476537 | fibroblast growth factor receptor 1 | NM_010206 | NP_034336 | UP TAC LA | | BC018128 | NP_075599 |
| BG072676 | FXYD domain-containing ion transport | NM_022004 | NP_071287 | UP TAC LA | | AK092198 | NP_071286 |
| | regulator 6 | | | | | | |
| AI838468 | gamma-aminobutyric acid (GABA-B) | NM_019439 | NP_062312 | UP TAC LA | | AJ225028 | NP_068705 |
| | receptor, 1 | | | | | | |
| AV057141 | gap junction membrane channel protein | NM_008124 | NP_032150 | | UP TAC LV | BF570961 | NP_000157 |
| | beta 1 | | | | | | |
| BG067028 | glycoprotein galactosyltransferase alpha 1, 3 | NM_010283 | NP_034413 | UP TAC LA | | | |
| AV033394 | glycoprotein m6b | NM_023122 | NP_075611 | UP TAC LA | | AK095657 | NP_005269 |
| AV085916 | GPI-anchored membrane protein 1 | BU611749 | | UP TAC LA | | | |
| BG063447 | guanine nucleotide binding protein, beta 1 | NM_008142 | NP_032168 | UP TAC LA | | AK123609 | NP_002065 |
| X00246 | histocompatibility 2, D region locus 1 | NM_010380 | NP_034510 | UP TAC LA | | | |
| BG064733 | HLS7-interacting protein kinase | NM_147201 | NP_671734 | UP TAC LA | | AK122664 | NP_037524 |
| AV010401 | integral membrane protein 2B | NM_008410 | NP_032436 | UP TAC LA | | BX537657 | NP_068839 |
| AV078295 | integrin alpha 6 | NM_008397 | NP_032423 | UP TAC LA | | X53586 | NP_000201 |
| BG074422 | integrin beta 1 (fibronectin receptor beta) | AK088016 | | UP TAC LA | | NM_002211 | NP_596867 |
| BF100414 | integrin beta 5 | NM_010580 | NP_034710 | UP TAC LA | | AK091595 | NP_002204 |
| AV006514 | interferon (alpha and beta) receptor 2 | NM_010509 | NP_034639 | UP TAC LA | | L41944 | NP_997468 |
| AV074586 | interleukin 17 receptor | BC037587 | | UP TAC LA | | | |
| BG070387 | interleukin 6 signal transducer | NM_010560 | NP_034690 | UP TAC LA | | BC071555 | NP_786943 |
| BG072624 | laminin, gamma 1 | BC032194 | NP_034813 | UP TAC LA | | NM_002293 | NP_002284 |
| AV054666 | leptin receptor | NM_175036 | NP_778201 | UP TAC LA | | | |
| BG075361 | low density lipoprotein receptor-related | NM_008512 | NP_032538 | UP TAC LA | | NM_002332 | NP_002323 |
| | protein 1 | | | | | | |
| AV162270 | lymphocyte antigen 6 complex, locus A | NM_027015 | NP_081291 | UP TAC LA | | | |
| BG065103 | lymphocyte antigen 6 complex, locus E | NM_008529 | NP_032555 | UP TAC LA | | BF969813 | NP_002337 |
| AV117035 | manic fringe homolog (Drosophila) | NM_008595 | NP_032621 | UP TAC LA | | U94352 | NP_002396 |
| AV026219 | mannosidase 1, alpha | NM_008548 | NP_032574 | UP TAC LA | | | |
| BG075377 | melanoma cell adhesion molecule | NM_023061 | NP_075548 | UP TAC LA | | NM_006500 | NP_006491 |
| BG072908 | membrane-bound transcription factor | NM_019709 | NP_062683 | UP TAC LA | | NM_003791 | NP_957720 |
| | protease, site 1 | | | | | | |
| AV025927 | Mus musculus, clone IMAGE: 5066061, | BC046959 | | UP TAC LA | | | |
| | mRNA, partial cds | | | | | | |
| AV057440 | Mus musculus, clone MGC: 27672 IMAGE: | NM_144852 | NP_659101 | UP TAC LA | | BC062565 | NP_004164 |
| | 4911158, mRNA, comp [symbol] | | | | | | |
| BG066621 | Mus musculus, Similar to pituitary | NM_145925 | NP_666037 | UP TAC LA | | | |
| | tumor-transforming 1 interacting [symbol] | | | | | | |
| BG064673 | Mus musculus, Similar to xylosylprotein | NM_146045 | NP_666157 | UP TAC LA | | AK022566 | NP_009186 |
| | beta1,4-galactosyltransfer [symbol] | | | | | | |
| BG072632 | myeloid-associated differentiation marker | NM_016969 | NP_058665 | UP TAC LA | | AF087882 | NP_612382 |
| BG072584 | myristoylated alanine rich protein kinase | NM_008538 | NP_032564 | UP TAC LA | | NM_002356 | NP_002347 |
| | C substrate | | | | | | |
| BG066563 | N-acetylated alpha-linked acidic | NM_028279 | NP_082555 | UP TAC LA | UP TAC LV | AK075390 | NP_005458 |
| | dipeptidase 2 | | | | | | |
| AV061081 | neural proliferation, differentiation | NM_008721 | NP_032747 | UP TAC LA | | AK054950 | NP_056207 |
| | and control gene 1 | | | | | | |
| BG074219 | neuroblastoma ras oncogene | NM_010937 | NP_035067 | UP TAC LA | | X02751 | NP_002515 |
| AI323974 | neuropilin | NM_008737 | NP_032763 | UP TAC LA | | | |
| BG063616 | nidogen 1 | NM_010917 | NP_035047 | UP TAC LA | | | |
| BF182158 | Notch gene homolog 1, (Drosophila) | NM_008714 | NP_032740 | UP TAC LA | | NM_017617 | NP_060087 |
| BF136770 | Notch gene homolog 3, (Drosophila) | NM_008716 | NP_032742 | UP TAC LA | | NM_000435 | NP_000426 |
| AV145718 | parathyroid hormone receptor | NM_011199 | NP_035329 | UP TAC LA | | AF495723 | NP_000307 |
| AV059520 | peptidylprolyl isomerase C-associated | NM_011150 | NP_035280 | UP TAC LA | | | |
| | protein | | | | | | |
| AV006019 | phosphatidylinositol glycan, class Q | NM_011822 | NP_035952 | UP TAC LA | | NM_004204 | NP_683721 |
| BG064035 | phosphoprotein enriched in astrocytes 15 | NM_008556 | NP_035193 | UP TAC LA | | NM_003768 | NP_003759 |
| AV112983 | platelet derived growth factor receptor, | NM_008809 | NP_032835 | UP TAC LA | | BC032224 | NP_002600 |
| | beta polypeptide | | | | | | |
| AV234882 | polycystic kidney disease 1 homolog | NM_013630 | NP_038658 | UP TAC LA | | L33243 | NP_000287 |
| AV009300 | procollagen, type IV, alpha 1 | J04694 | | UP TAC LA | | NM_001845 | NP_001836 |
| BG074718 | procollagen, type IV, alpha 2 | J04695 | | UP TAC LA | | NM_001846 | NP_001837 |
| AV025665 | prostaglandin-endoperoxide synthase 2 | NM_011198 | NP_035328 | UP TAC LA | | NM_000963 | NP_000954 |
| BG067870 | protein kinase C, delta | NM_011103 | NP_035233 | UP TAC LA | | NM_006254 | NP_997704 |
| BG070083 | protein tyrosine phosphatase, receptor | NM_011212 | NP_035342 | UP TAC LA | | BX648180 | NP_569119 |
| | type, E | | | | | | |
| BG074663 | protein tyrosine phosphatase, receptor | NM_011218 | NP_035348 | UP TAC LA | | NM_002850 | NP_570925 |
| | type, S | | | | | | |
| AI893212 | proteolipid protein 2 | NM_019755 | NP_062729 | UP TAC LA | | BF214130 | NP_002659 |
| BG073000 | protocadherin 13 | NM_033576 | NP_291054 | UP TAC LA | | | |
| AV086128 | regulator of G-protein signaling 19 | NM_018771 | NP_061241 | UP TAC LA | | NM_005716 | NP_974223 |
| | interacting protein 1 | | | | | | |
| AU040596 | regulator of G-protein signaling 3 | NM_019492 | NP_062365 | UP TAC LA | | AK128127 | NP_652760 |
| AV084219 | reticulon 4 | NM_024226 | NP_077188 | UP TAC LA | | NM_020532 | NP_997404 |
| BG073341 | retinal short-chain dehydrogenase/ | NM_011303 | NP_035433 | UP TAC LA | | BX648476 | NP_004744 |
| | reductase 1 | | | | | | |
| AV024396 | reversion-inducing-cysteine-rich | NM_016678 | NP_057887 | UP TAC LA | | BX648668 | NP_066934 |
| | protein with kazal motifs | | | | | | |
| BG063638 | ribosome binding protein 1 | AK019964 | NP_598329 | UP TAC LA | | AB037819 | NP_004578 |
| AW538766 | RIKEN cDNA 0610013I17 gene | NM_029789 | NP_084065 | UP TAC LA | | NM_012432 | NP_036564 |
| AV133782 | RIKEN cDNA 0610039A15 gene | NM_175101 | NP_780310 | UP TAC LA | | | |
| AV007276 | RIKEN cDNA 1110003M08 gene | AK090329 | | UP TAC LA | | AK124975 | NP_005818 |
| AV058524 | RIKEN cDNA 1110007A14 gene | NM_025841 | NP_080117 | UP TAC LA | | AK093917 | NP_006845 |
| AV133706 | RIKEN cDNA 1110059L23 gene | NM_134255 | NP_599016 | UP TAC LA | | AL833001 | NP_068586 |
| AV086520 | RIKEN cDNA 1200003O06 gene | NM_025813 | NP_080089 | UP TAC LA | | | |
| BG064285 | RIKEN cDNA 1200013F24 gene | NM_025822 | NP_080098 | UP TAC LA | | | |
| AV088097 | RIKEN cDNA 1200015A22 gene | NM_028766 | NP_083042 | UP TAC LA | | | |
| BG074142 | RIKEN cDNA 1300012G16 gene | NM_023625 | NP_076114 | UP TAC LA | | | |
| AV086327 | RIKEN cDNA 2310008D10 gene | NM_025858 | NP_080657 | UP TAC LA | | | |
| AV087181 | RIKEN cDNA 2310028N02 gene | NM_025864 | NP_080140 | UP TAC LA | | | |
| AV085104 | RIKEN cDNA 2410001H17 gene | NM_025889 | NP_080165 | UP TAC LA | | | |
| BG067332 | RIKEN cDNA 2610002H11 gene | NM_133721 | NP_598482 | UP TAC LA | | BX647350 | NP_002198 |
| BG073064 | RIKEN cDNA 2610027H02 gene | BC027791 | | UP TAC LA | | | |
| AV061276 | RIKEN cDNA 5031406P05 gene | NM_026669 | NP_080945 | UP TAC LA | | AK130050 | NP_003208 |
| AV020551 | RIKEN cDNA 5730403E06 gene | NM_027439 | NP_081715 | UP TAC LA | | | |
| AV016743 | RIKEN cDNA 5730414C17 gene | NM_133680 | NP_598441 | UP TAC LA | | | |
| AV085966 | RIKEN cDNA 6720474K14 gene | NM_175414 | NP_780623 | UP TAC LA | | | |
| BG072850 | sarcoglycan, epsilon | NM_011360 | NP_035490 | UP TAC LA | | NM_003919 | NP_003910 |
| AV087531 | scavenger receptor class B1 | NM_016741 | NP_058021 | UP TAC LA | | AK023485 | NP_005496 |
| AV021712 | secreted frizzled-related sequence protein 2 | NM_009144 | NP_033170 | UP TAC LA | | NM_003013 | NP_003004 |
| AV062462 | serine palmitoyltransferase, long chain | NM_009269 | NP_033295 | UP TAC LA | | NM_006415 | NP_847894 |
| | base subunit 1 | | | | | | |
| D16106 | sialyltransferase 1 (beta-galactoside | NM_145933 | NP_666045 | UP TAC LA | | | |
| | alpha-2,6-sialyltransferase) | | | | | | |
| AI385650 | sialyltransferase 4C (beta-galactosidase | NM_009178 | NP_033204 | UP TAC LA | | AK128605 | NP_006269 |
| | alpha-2,3-sialytransferase [symbol] | | | | | | |
| AV093704 | small EDRK-rich factor 2 | AK044479 | | | UP TAC LV | | |
| BG075739 | solute carrier family 29 (nucleoside | NM_022880 | NP_075018 | UP TAC LA | | AK090615 | NP_004946 |
| | transporters), member 1 | | | | | | |
| AA499432 | sprouty homolog 4 (Drosophila) | NM_011898 | NP_036028 | UP TAC LA | | AF227516 | NP_112226 |
| AV074505 | surfeit gene 4 | NM_011512 | NP_035642 | UP TAC LA | | NM_033161 | NP_149351 |
| AV111434 | transient receptor protein 2 | BF583628 | | UP TAC LA | | BM701565 | NP_852667 |
| AV083947 | transmembrane domain protein regulated | NM_011906 | NP_036036 | UP TAC LA | | | |
| | in adipocytes 40 kDa | | | | | | |
| AA023493 | transmembrane protein with EGF-like and | AK079633 | | UP TAC LA | | NM_003692 | NP_003683 |
| | two follistatin-like domai [symbol] | | | | | | |
| L26349 | tumor necrosis factor receptor superfamily, | NM_011609 | NP_035739 | UP TAC LA | | NM_001065 | NP_001056 |
| | member 1a | | | | | | |
| AV024570 | tumor necrosis factor, alpha-induced | NM_009395 | NP_033421 | UP TAC LA | | BC003694 | NP_066960 |
| | protein 1 (endothelial) | | | | | | |
| BG062994 | UDP-GlcNAc: betaGal | NM_016888 | NP_058584 | UP TAC LA | | BC047933 | NP_150274 |
| | beta-1,3-N-acetylglucosaminyltransferase 1 [symbol] | | | | | | |
| BG073697 | UDP-glucuronate decarboxylase 1 | NM_026430 | NP_080706 | UP TAC LA | | BC035177 | NP_079352 |
| BG064510 | vanilloid receptor-like protein 1 | NM_011706 | NP_035836 | UP TAC LA | | AK126996 | NP_057197 |
| BE376968 | vascular endothelial growth factor C | NM_009506 | NP_033532 | UP TAC LA | | NM_005429 | NP_005420 |
| AV103195 | zinc finger protein 36 | NM_133786 | NP_598547 | UP TAC LA | | NM_005496 | NP_005487 |

TABLE III
Table III Genes of Use in Serologic Assays and/or Imaging Studies
Annotated Extracellular and Antigen genes Upregulated in TAC tissues - 169 Unique genes
One example for each gene - Passed stringent SAM criteria

| | | | | | | Human Homolog Information | |
|---|---|---|---|---|---|---|---|
| Mouse Gene Information | | | | | | Human | Human |
| Gene ID | Gene Description | UGRepAcc | LLReProtA | Up TAC LA | Up TAC LV | UGRepA [symbol] | LLRep [symbol] |
| AI841353 | a disintegrin and metalloproteinase | NM_009614 | NP_033744 | UP TAC LA | | AY560601 | NP_997080 |
| | domain 15 (metargidin) | | | | | | |
| AV077899 | actin, alpha 2, smooth muscle, aorta | AK002886 | | | UP TAC LV | | |
| BG072752 | actin, gamma, cytoplasmic | NM_013798 | NP_038826 | | UP TAC LV | | |
| BG063167 | adenylate cyclase 7 | NM_007406 | NP_031432 | UP TAC LA | UP TAC LV | D25538 | NP_001105 |
| BG074747 | alpha glucosidase 2, alpha neutral | NM_008060 | NP_032086 | UP TAC LA | | | |
| | subunit | | | | | | |
| AV070218 | amyloid beta (A4) precursor-like | NM_009691 | NP_033821 | UP TAC LA | | BX647107 | NP_001633 |
| | protein 2 | | | | | | |
| AV070419 | antigen identified by monoclonal | NM_010818 | NP_034948 | UP TAC LA | | BC022522 | NP_005935 |
| | antibody MRC OX-2 | | | | | | |
| AV025941 | aquaporin 1 | NM_007472 | NP_031498 | UP TAC LA | | NM_198098 | NP_932766 |
| U34920 | ATP-binding cassette, sub-family G | NM_009593 | NP_033723 | UP TAC LA | | NM_207630 | NP_997513 |
| | (WHITE), member 1 | | | | | | |
| AV104097 | basigin | BI106083 | | UP TAC LA | | NM_001728 | NP_940993 |
| AV087921 | benzodiazepine receptor, peripheral | NM_009775 | NP_033905 | UP TAC LA | | BX537892 | NP_009295 |
| X01838 | beta-2 microglobulin | NM_009735 | NP_033865 | UP TAC LA | | AK022379 | NP_004039 |
| AV170826 | biglycan | NM_007542 | NP_031568 | UP TAC LA | | BC004244 | NP_001702 |
| AA498724 | bone morphogenetic protein 4 | NM_007554 | NP_031580 | UP TAC LA | | NM_001202 | NP_570912 |
| D16250 | bone morphogenetic protein receptor, | BC042611 | NP_033888 | UP TAC LA | | NM_004329 | NP_004320 |
| | type 1A | | | | | | |
| AV089105 | calcium binding protein, intestinal | NM_009787 | NP_033917 | UP TAC LA | | | |
| X52886 | cathepsin D | NM_009983 | NP_034113 | UP TAC LA | | NM_001909 | NP_001900 |
| AV171867 | CD 81 antigen | NM_133655 | NP_598416 | UP TAC LA | | BM810055 | NP_004347 |
| AV062071 | CD24a antigen | NM_009846 | NP_033976 | UP TAC LA | | | |
| AI893233 | CD34 antigen | NM_133654 | NP_598415 | UP TAC LA | | BX640941 | NP_001764 |
| AI838302 | Cd63 antigen | NM_007653 | NP_031679 | UP TAC LA | | BM701371 | NP_001771 |
| BG073140 | CD8 antigen, beta chain | NM_009858 | NP_033988 | UP TAC LA | | | |
| AI325851 | CD97 antigen | NM_011925 | NP_036055 | UP TAC LA | | NM_078481 | NP_510966 |
| AV109555 | cellular retinoic acid binding protein I | AK090130 | | UP TAC LA | | NM_212482 | NP_997647 |
| BG067569 | coagulation factor II (thrombin) receptor | NM_010169 | NP_03429 | UP TAC LA | | NM_001992 | NP_001983 |
| AV149987 | cystatin C | NM_009976 | NP_034106 | UP TAC LA | | BX647523 | NP_000090 |
| BG074174 | DNA segment, Chr 6, Wayne State | NM_138587 | NP_613053 | UP TAC LA | | | |
| | University 176, expressed | | | | | | |
| AV104157 | dolichyl-di-phosphooligosaccharide- | NM_007838 | NP_031864 | UP TAC LA | | NM_005216 | NP_005207 |
| | protein glycotransferase | | | | | | |
| AV083262 | dystonin | NM_134448 | NP_604443 | | UP TAC LV | NM_183380 | NP_899236 |
| BG065640 | ectonucleotide pyrophosphatase/ | NM_008813 | NP_032839 | UP TAC LA | | NM_006208 | NP_006199 |
| | phosphodiesterase 1 | | | | | | |
| AV019210 | elastin | NM_007925 | NP_031951 | UP TAC LA | | BX537939 | NP_000492 |
| AV066211 | ELAV (embryonic lethal, abnormal | NM_010485 | NP_034615 | UP TAC LA | | NM_001419 | NP_001410 |
| | vision, Drosophila)-like 1 (H [symbol] | | | | | | |
| AA646363 | endoglin | NM_007932 | NP_031958 | UP TAC LA | | NM_000118 | NP_000109 |
| AV104213 | endothelial cell-selective adhesion | NM_027102 | NP_081378 | UP TAC LA | | | |
| | molecule | | | | | | |
| AI838613 | epithelial membrane protein 1 | | | UP TAC LA | UP TAC LV | NM_001423 | NP_001414 |
| AV011166 | EST | NM_080463 | NP_536711 | UP TAC LA | | AF375884 | NP_758436 |
| AV087039 | EST | NM_008885 | NP_032911 | UP TAC LA | | NM_000304 | NP_696997 |
| AV140901 | EST | NM_010368 | NP_034498 | UP TAC LA | | | |
| AW537378 | EST | | | SAM | UP TAC LV | | |
| | | | | DOWN | | | |
| AW547864 | EST | | | | UP TAC LV | | |
| U20156 | EST | | | UP TAC LA | UP TAC LV | BQ056329 | NP_002406 |
| AV087499 | EST, Moderately similar to A57474 | NM_007899 | NP_031925 | UP TAC LA | | AK097205 | NP_073155 |
| | extracellular matrix protein [symbol] | | | | | | |
| AI851039 | ESTs, Weakly similar to D2045.2.p | AK038775 | | | UP TAC LV | | |
| | [Caenorhabditis elegans] [ [symbol] | | | | | | |
| AV059438 | ets variant gene 6 (TEL oncogene) | BC009120 | | | UP TAC LV | | |
| BG064180 | expressed sequence AA408225 | NM_009868 | NP_033998 | UP TAC LA | | NM_001795 | NP_001786 |
| AV059924 | expressed sequence AA986889 | NM_134102 | NP_598863 | UP TAC LA | | BX647516 | NP_056984 |
| AV103290 | expressed sequence AL024047 | NM_134151 | NP_598912 | UP TAC LA | | AK125213 | NP_003671 |
| BG072998 | expressed sequence AU018638 | NM_008524 | NP_032550 | | UP TAC LV | BG114678 | NP_002336 |
| AV037769 | expressed sequence AU022549 | NM_007904 | NP_031930 | UP TAC LA | | NM_000115 | NP_003982 |
| AV087220 | expressed sequence AW146116 | NM_133352 | NP_835359 | UP TAC LA | | | |
| BG073479 | expressed sequence AW229038 | NM_133918 | NP_598679 | UP TAC LA | | AL050138 | NP_008977 |
| BG070007 | expressed sequence AW494241 | BC040467 | | | UP TAC LV | | |
| C79946 | expressed sequence C79946 | AK080023 | | UP TAC LA | UP TAC LV | | |
| AV085019 | extracellular matrix protein 1 | NM_007899 | NP_031925 | UP TAC LA | | AK097205 | NP_073155 |
| AW476537 | fibroblast growth factor receptor 1 | NM_010206 | NP_034336 | UP TAC LA | | BC018128 | NP_075599 |
| AA673390 | fibronectin 1 | AK090130 | | UP TAC LA | | NM_212482 | NP_997647 |
| BG073227 | fibulin 2 | NM_007992 | NP_032018 | UP TAC LA | | AY130459 | NP_{—} |
| | | | | | | | 001004019 |
| AV059445 | FK506 binding protein 9 | NM_012056 | NP_036186 | UP TAC LA | | AK075331 | NP_009201 |
| BG063294 | follistatin-like 3 | NM_031380 | NP_113557 | UP TAC LA | | BC005839 | NP_005851 |
| AV083596 | four and a half LIM domains 1 | NM_010211 | NP_034341 | | UP TAC LV | AK122708 | NP_001440 |
| AV086002 | FXYD domain-containing ion | NM_022004 | NP_071287 | UP TAC LA | | AK092198 | NP_071286 |
| | transport regulator 6 | | | | | | |
| AV057141 | gap junction membrane channel | NM_008124 | NP_032150 | | UP TAC LV | BF570961 | NP_000157 |
| | protein beta 1 | | | | | | |
| AV073997 | glucose regulated protein, 58 kDa | NM_007952 | NP_031978 | UP TAC LA | | AK075455 | NP_005304 |
| AV001464 | granulin | NM_008175 | NP_032201 | UP TAC LA | | NM_002087 | NP_002078 |
| AV134035 | granulin | NM_008175 | NP_032201 | UP TAC LA | | NM_002087 | NP_002078 |
| AV223941 | heat shock protein, 70 kDa 3 | M12571 | | SAM | UP TAC LV | NM_005345 | NP_005336 |
| | | | | DOWN | | | |
| AW551778 | heterogeneous nuclear | NM_016884 | NP_058580 | UP TAC LA | UP TAC LV | AK126950 | NP_112604 |
| | ribonucleoprotein C | | | | | | |
| X00246 | histocompatibility 2, D region locus 1 | NM_010380 | NP_034510 | UP TAC LA | | | |
| AV084844 | immunoglobulin superfamily containing | NM_012043 | NP_036173 | UP TAC LA | | NM_005545.3 | NP_005536.1 |
| | leucine-rich repeat | | | | | | |
| AV012617 | insulin-like growth factor binding | NM_010518 | NP_034648 | UP TAC LA | | NM_000599 | NP_000590 |
| | protein 5 | | | | | | |
| BG074422 | integrin beta 1 (fibronectin receptor | AK088016 | | UP TAC LA | | NM_002211 | NP_596867 |
| | beta) | | | | | | |
| BG073319 | integrin beta 4 binding protein | NM_010579 | NP_034709 | | UP TAC LV | BQ278496 | NP_852134 |
| BF100414 | integrin beta 5 | NM_010580 | NP_034710 | UP TAC LA | | AK091595 | NP_002204 |
| AV006514 | interferon (alpha and beta) receptor 2 | NM_010509 | NP_034639 | UP TAC LA | | L41944 | NP_997468 |
| BG070387 | interleukin 6 signal transducer | NM_010560 | NP_034690 | UP TAC LA | | BC071555 | NP_786943 |
| BG072624 | laminin, gamma 1 | BC032194 | NP_034813 | UP TAC LA | | NM_002293 | NP_002284 |
| AV007183 | latent transforming growth factor | NM_023912 | NP_076401 | UP TAC LA | | AK024477 | NP_066548 |
| | beta binding protein 3 | | | | | | |
| BG071948 | low density lipoprotein receptor-related | NM_008512 | NP_032538 | | UP TAC LV | NM_002332 | NP_002323 |
| | protein 1 | | | | | | |
| AV162270 | lymphocyte antigen 6 complex, locus A | NM_027015 | NP_081291 | UP TAC LA | | NM_001030 | NP_001021 |
| BG065103 | lymphocyte antigen 6 complex, locus E | NM_008529 | NP_032555 | UP TAC LA | | BF969813 | NP_002337 |
| AA098349 | lysyl oxidase-like | AK078512 | | UP TAC LA | | BC068542 | NP_005567 |
| AV117035 | manic fringe homolog (Drosophila) | NM_008595 | NP_032621 | UP TAC LA | | U94352 | NP_002396 |
| AV156534 | matrilin 2 | NM_016762 | NP_058042 | UP TAC LA | | BX648291 | NP_085072 |
| AI838311 | matrix metalloproteinase 2 | NM_008610 | NP_032636 | | UP TAC LV | AL832088 | NP_004521 |
| AV015188 | matrix metalloproteinase 23 | NM_011985 | NP_036115 | UP TAC LA | | | |
| BG075377 | melanoma cell adhesion molecule | NM_023061 | NP_075548 | UP TAC LA | | NM_006500 | NP_006491 |
| BG072908 | membrane-bound transcription | NM_019709 | NP_062683 | UP TAC LA | | NM_003791 | NP_957720 |
| | factor protease, site 1 | | | | | | |
| BG074344 | mesothelin | NM_018857 | NP_061345 | UP TAC LA | | BC003512 | NP_037536 |
| AV113097 | microfibrillar associated | NM_015776 | NP_056591 | UP TAC LA | | NM_003480 | NP_003471 |
| | protein 5 | | | | | | |
| AV094498 | milk fat globule-EGF factor 8 protein | NM_008594 | NP_032620 | UP TAC LA | | AK092157 | NP_005919 |
| AV085874 | Mus musculus | NM_139297 | NP_647458 | | UP TAC LV | BX537559 | NP_006750 |
| | uridindiphosphoglucosepyrophosphorylase | | | | | | |
| | 2 (U [symbol] | | | | | | |
| BG065584 | Mus musculus, clone IMAGE: 3589087, | BF124761 | | | UP TAC LV | | |
| | mRNA, partial cds | | | | | | |
| BG066621 | Mus musculus, Similar to pituitary | NM_145925 | NP_666037 | UP TAC LA | | | |
| | tumor-transforming 1 interac [symbol] | | | | | | |
| BG066563 | N-acetylated alpha-linked acidic | NM_028279 | NP_082555 | UP TAC LA | UP TAC LV | AK075390 | NP_005458 |
| | dipeptidase 2 | | | | | | |
| AV061081 | neural proliferation, differentiation | NM_008721 | NP_032747 | UP TAC LA | | AK054950 | NP_056207 |
| | and control gene 1 | | | | | | |
| AI325886 | neuroblastoma, suppression of | NM_008675 | NP_032701 | UP TAC LA | | NM_182744 | NP_877421 |
| | tumorigenicity 1 | | | | | | |
| AI323974 | neuropilin | NM_008737 | NP_032763 | UP TAC LA | | | |
| BG063616 | nidogen 1 | NM_010917 | NP_035047 | UP TAC LA | | | |
| BG072810 | Niemann Pick type C2 | NM_023409 | NP_075898 | UP TAC LA | | BQ896617 | NP_006423 |
| BF182158 | Notch gene homolog 1, (Drosophila) | NM_008714 | NP_032740 | UP TAC LA | | NM_017617 | NP_060087 |
| BF136770 | Notch gene homolog 3, (Drosophila) | NM_008716 | NP_032742 | UP TAC LA | | NM_000435 | NP_000426 |
| AV084876 | osteoblast specific factor 2 | NM_015784 | NP_056599 | UP TAC LA | | | |
| | (fasciclin I-like) | | | | | | |
| BG074915 | parotid secretory protein | NM_172261 | NP_758465 | UP TAC LA | | AL713642 | NP_115984 |
| AV059520 | peptidylprolyl isomerase C-associated | NM_011150 | NP_035280 | UP TAC LA | | | |
| | protein | | | | | | |
| AV112983 | platelet derived growth factor | NM_008809 | NP_032835 | UP TAC LA | | BC032224 | NP_002600 |
| | receptor, beta polypeptide | | | | | | |
| AI327133 | polydomain protein | NM_022814 | NP_073725 | UP TAC LA | | | |
| BG073284 | prion protein dublet | NM_023043 | NP_075530 | | UP TAC LV | NM_012409 | NP_036541 |
| AV084561 | procollagen C-proteinase enhancer protein | NM_008788 | NP_032814 | UP TAC LA | UP TAC LV | BM994449 | NP_002584 |
| AV009300 | procollagen, type IV, alpha 1 | J04694 | | UP TAC LA | | NM_001845 | NP_001836 |
| AV010312 | procollagen, type IV, alpha 2 | J04695 | | UP TAC LA | | NM_001846 | NP_001837 |
| AV013988 | procollagen, type VI, alpha 1 | NM_009933 | NP_034063 | UP TAC LA | | NM_001848 | NP_001839 |
| BG075864 | procollagen, type VI, alpha 2 | NM_146007 | NP_666119 | UP TAC LA | | AK128695 | NP_478055 |
| AV015595 | procollagen, type XV | NM_009928 | NP_034058 | UP TAC LA | | NM_001855 | NP_001846 |
| AW548258 | procollagen-proline, 2-oxoglutarate | BC009654 | | UP TAC LA | | BX648829 | NP_000908 |
| | 4-dioxygenase (proline 4-h [symbol] | | | | | | |
| BG069745 | proline arginine-rich end leucine-rich | NM_054077 | NP_473418 | UP TAC LA | | NM_002725 | NP_958505 |
| | repeat | | | | | | |
| BG073729 | prolyl 4-hydroxylase, beta polypeptide | J05185 | | UP TAC LA | | J02783 | NP_000909 |
| BG073750 | prolyl 4-hydroxylase, beta polypeptide | J05185 | | UP TAC LA | | J02783 | NP_000909 |
| AV025665 | prostaglandin-endoperoxide synthase 2 | NM_011198 | NP_035328 | UP TAC LA | | NM_000963 | NP_000954 |
| BG070083 | protein tyrosine phosphatase, receptor | NM_011212 | NP_035342 | UP TAC LA | | BX648180 | NP_569119 |
| | type, E | | | | | | |
| BG074663 | protein tyrosine phosphatase, receptor | NM_011218 | NP_035348 | UP TAC LA | | NM_002850 | NP_570925 |
| | type, S | | | | | | |
| BG073341 | retinal short-chain dehydrogenase/ | NM_011303 | NP_035433 | UP TAC LA | | BX648476 | NP_004744 |
| | reductase 1 | | | | | | |
| AV083867 | retinoid-inducible serine caroboxypetidase | NM_029023 | NP_083299 | UP TAC LA | | | |
| AA087526 | retinol binding protein 1, cellular | NM_011254 | NP_035384 | | UP TAC LV | BF508021 | NP_002890 |
| AV024396 | reversion-inducing-cysteine-rich | NM_016678 | NP_057887 | UP TAC LA | | BX648668 | NP_066934 |
| | protein with kazal motifs | | | | | | |
| AV140189 | RIKEN cDNA 0610040B21 gene | NM_025334 | NP_079610 | UP TAC LA | | | |
| AV007276 | RIKEN cDNA 1110003M08 gene | AK090329 | | UP TAC LA | | AK124975 | NP_005818 |
| AV083352 | RIKEN cDNA 1110007F23 gene | NM_029568 | NP_083844 | UP TAC LA | | | |
| AV015246 | RIKEN cDNA 1110054M18 gene | NM_175132 | NP_780341 | | UP TAC LV | | |
| BG074142 | RIKEN cDNA 1300012G16 gene | NM_023625 | NP_076114 | UP TAC LA | | | |
| AI838568 | RIKEN cDNA 1300018J16 gene | NM_029092 | NP_083368 | UP TAC LA | UP TAC LV | | |
| AV058250 | RIKEN cDNA 1810049K24 gene | NM_030209 | NP_084485 | UP TAC LA | | | |
| AI322274 | RIKEN cDNA 2410002J21 gene | AK033091 | | | UP TAC LV | | |
| AI851067 | RIKEN cDNA 2510010F10 gene | NM_175833 | NP_787027 | | UP TAC LV | | |
| AV111526 | RIKEN cDNA 2610002H11 gene | NM_133721 | NP_598482 | UP TAC LA | | BX647350 | NP_002198 |
| AV050682 | RIKEN cDNA 2700083B06 gene | NM_026531 | NP_080807 | UP TAC LA | UP TAC LV | | |
| AV133755 | RIKEN cDNA 2810002E22 gene | NM_133859 | NP_598620 | UP TAC LA | | | |
| AV053955 | RIKEN cDNA 3110023E09 gene | NM_026522 | NP_080798 | UP TAC LA | | | |
| AV016743 | RIKEN cDNA 5730414C17 gene | NM_133680 | NP_598441 | UP TAC LA | | | |
| BG072850 | sarcoglycan, epsilon | NM_011360 | NP_035490 | UP TAC LA | | NM_003919 | NP_003910 |
| AW988741_2 | secreted acidic cysteine rich glycoprotein | | | UP TAC LA | | AK126525 | NP_003109 |
| AV021712 | secreted frizzled-related sequence | NM_009144 | NP_033170 | UP TAC LA | | NM_003013 | NP_003004 |
| | protein 2 | | | | | | |
| BG074382 | sema domain, immunoglobulin domain | NM_011349 | NP_035479 | UP TAC LA | | U38276 | NP_004177 |
| | (Ig), short basic domain [symbol] | | | | | | |
| AV022379 | serine (or cysteine) proteinase inhibitor, | NM_011340 | NP_035470 | UP TAC LA | | BM918904 | NP_002606 |
| | clade F (alpha-2 antipl [symbol] | | | | | | |
| AV093463 | serine (or cysteine) proteinase inhibitor, | NM_009825 | NP_033955 | UP TAC LA | | AK122936 | NP_001226 |
| | clade H (heat shock pr [symbol] | | | | | | |
| AV052090 | serine (or cysteine) proteinase inhibitor, | NM_009250 | NP_033276 | UP TAC LA | | BC018043 | NP_005016 |
| | clade I (neuroserpin), [symbol] | | | | | | |
| AI385650 | sialyltransferase 4C (beta-galactosidase | NM_009178 | NP_033204 | UP TAC LA | | AK128605 | NP_006269 |
| | alpha-2,3-sialytransfe [symbol] | | | | | | |
| AV093704 | small EDRK-rich factor 2 | AK044479 | | | UP TAC LV | | |
| AV109513 | stromal cell derived factor 1 | NM_013655 | NP_068350 | UP TAC LA | | BX647204 | NP_954637 |
| AV048780 | stromal cell derived factor 4 | NM_011341 | NP_035471 | UP TAC LA | | | |
| U38261 | superoxide dismutase 3, extracellular | NM_011435 | NP_035565 | UP TAC LA | | NM_003102 | NP_003093 |
| AV070805 | thymic stromal-derived lymphopoietin, | NM_016715 | NP_057924 | UP TAC LA | | | |
| | receptor | | | | | | |
| AV057827 | torsin family 3, member A | NM_023141 | NP_075630 | UP TAC LA | | NM_022371 | NP_071766 |
| AA068104 | transforming growth factor, beta 2 | NM_009367 | NP_033393 | UP TAC LA | | M19154 | NP_003229 |
| L26349 | tumor necrosis factor receptor | NM_011609 | NP_035739 | UP TAC LA | | NM_001065 | NP_001056 |
| | superfamily, member 1a | | | | | | |
| BE376968 | vascular endothelial growth factor C | NM_009506 | NP_033532 | UP TAC LA | | NM_005429 | NP_005420 |

TABLE IV
Table IV Genes of Use in Metabolic Assays
Annotated Metabolism Genes Downregulated in TAC tissues - 109 Unique genes
One example for each gene - Passed stringent SAM criteria

| Mouse Gene Information | | | | | | | |
|---|---|---|---|---|---|---|---|
| Gene Name | Gene Description | UGRepAcc | LLRepProtA | Down TAC LA | Down TAC LV | UGRepAcc | LLRepProtAcc |
| BG066890 | **DNA segment, Chr 13, ERATO | NM_007749 | NP_031775 | DOWN TAC LA | | BI118114 | NP_001858 |
| | Doi 332, expressed | | | | | | |
| BG062980 | **DNA segment, Chr 2, Wayne State | U37501 | | DOWN TAC LA | | NM_005560 | NP_005551 |
| | University 85, expressed | | | | | | |
| AV025301 | 2,4-dienoyl CoA reductase 1, | NM_026172 | NP_080448 | | DOWN TAC LV | BM920635 | NP_001350 |
| | mitochondrial | | | | | | |
| AV029241 | acetyl-Coenzyme A dehydrogenase, | NM_007381 | NP_031407 | DOWN TAC LA | DOWN TAC LV | BC039063 | NP_001599 |
| | long-chain | | | | | | |
| AI840666 | acetyl-Coenzyme A dehydrogenase, | NM_007382 | NP_031408 | DOWN TAC LA | DOWN TAC LV | NM_000016 | NP_000007 |
| | medium chain | | | | | | |
| AV004604 | acetyl-Coenzyme A dehydrogenase, | NM_007383 | NP_031409 | | DOWN TAC LV | AK057021 | NP_000008 |
| | short chain | | | | | | |
| AI839605 | acyl-Coenzyme A dehydrogenase, | NM_017366 | NP_059062 | DOWN TAC LA | | AK097243 | NP_000009 |
| | very long chain | | | | | | |
| AF006688 | acyl-Coenzyme A oxidase 1, | NM_015729 | NP_056544 | | DOWN TAC LV | BC008767 | NP_009223 |
| | palmitoyl | | | | | | |
| U07235 | aldehyde dehydrogenase 2, | NM_009656 | NP_033786 | | DOWN TAC LV | AL832043 | NP_000681 |
| | mitochondrial | | | | | | |
| AV006235 | ATPase, Ca++ transporting, cardiac | NM_009722 | NP_033852 | | DOWN TAC LV | BX648282 | NP_733765 |
| | muscle, slow twitch 2 | | | | | | |
| BG074044 | ATPase, Ca++ transporting, cardiac | NM_009722 | NP_033852 | DOWN TAC LA | DOWN TAC LV | BX648282 | NP_733765 |
| | muscle, slow twitch 2 | | | | | | |
| AI837797 | ATPase, Ca++ transporting, cardiac | NM_009722 | NP_033852 | DOWN TAC LA | | BX648282 | NP_733765 |
| | muscle, slow twitch 2 | | | | | | |
| AV095181 | AU RNA binding protein/ | NM_016709 | NP_057918 | DOWN TAC LA | | AK124142 | NP_001689 |
| | enoyl-coenzyme A hydratase | | | | | | |
| AI323918 | branched chain ketoacid | NM_007533 | NP_031559 | | DOWN TAC LV | BF206112 | NP_000700 |
| | dehydrogenase E1, alpha polypeptide [symbol] | | | | | | |
| AV014385 | carbonic anhydrase 14 | NM_146104 | NP_666216 | DOWN TAC LA | DOWN TAC LV | | |
| AV170903 | carbonic anhydrase 14 | NM_146104 | NP_666216 | | DOWN TAC LV | | |
| AI323923 | carbonyl reductase 1 | NM_007620 | NP_031646 | DOWN TAC LA | | BM810059 | NP_001748 |
| AV006197 | carnitine palmitoyltransferase 2 | NM_009949 | NP_034079 | DOWN TAC LA | DOWN TAC LV | NM_000098 | NP_000089 |
| AV093569 | copper chaperone for superoxide | NM_016892 | NP_058588 | DOWN TAC LA | | BM543741 | NP_005116 |
| | dismutase | | | | | | |
| AV085004 | creatine kinase, mitochondrial 2 | AK009042 | | DOWN TAC LA | | NM_001825 | NP_001816 |
| AV005997 | cytochrome c oxidase, subunit IVa | NM_009941 | NP_034071 | DOWN TAC LA | | AK027136 | NP_001852 |
| AV095075 | cytochrome c oxidase, subunit Va | NM_007747 | NP_031773 | | DOWN TAC LV | BM911641 | NP_004246 |
| AV088644 | cytochrome c oxidase, subunit Vb | NM_009942 | NP_034072 | DOWN TAC LA | | BM912880 | NP_001853 |
| AV001082 | cytochrome c oxidase, subunit | NM_009943 | NP_034073 | DOWN TAC LA | DOWN TAC LV | BM712970 | NP_005196 |
| | VI a, polypeptide 2 | | | | | | |
| AV149855 | cytochrome c oxidase, subunit VIc | NM_053071 | NP_444301 | DOWN TAC LA | DOWN TAC LV | AK128382 | NP_004365 |
| AV086493 | cytochrome c oxidase, subunit VIIa 1 | NM_009944 | NP_034074 | DOWN TAC LA | | BM726594 | NP_001855 |
| AV133935 | cytochrome c oxidase, subunit VIIa 3 | NM_009945 | NP_034075 | DOWN TAC LA | DOWN TAC LV | BF210089 | NP_001856 |
| BG063960 | cytochrome c oxidase, subunit VIIc | NM_007749 | NP_031775 | DOWN TAC LA | | BI118114 | NP_001858 |
| AV086888 | cytochrome c, somatic | NM_007808 | NP_031834 | DOWN TAC LA | | NM_018947 | NP_061820 |
| AV093672 | cytochrome c-1 | NM_025567 | NP_079843 | DOWN TAC LA | | BF569085 | NP_001907 |
| AV095067 | DNA segment, Chr 18, Wayne | NM_138600 | NP_613066 | | DOWN TAC LV | AK092507 | NP_001173 |
| | State University 181, expressed [symbol] | | | | | | |
| AV083353 | dodecenoyl-Coenzyme A delta | NM_010023 | NP_034153 | DOWN TAC LA | DOWN TAC LV | BQ277959 | NP_001910 |
| | isomerase (3,2 trans-enoyl-Coe [symbol] | | | | | | |
| BG074113 | enoyl coenzyme A hydratase 1, | NM_016772 | NP_058052 | DOWN TAC LA | | AK126566 | NP_001389 |
| | peroxisomal | | | | | | |
| AU022217 | epoxide hydrolase 2, cytoplasmic | NM_007940 | NP_031966 | | DOWN TAC LV | AK094393 | NP_001970 |
| BG067242 | ESTs | BE988802 | | DOWN TAC LA | | NM_002660 | NP_877963 |
| AV006522 | ESTs | NM_028545 | NP_082821 | DOWN TAC LA | | | |
| AV095205 | eukaryotic translation initiation | NM_010121 | NP_034251 | DOWN TAC LA | | NM_004836 | NP_004827 |
| | factor 2 alpha kinase 3 | | | | | | |
| AV109470 | expressed sequence AA959857 | BC048412 | | DOWN TAC LA | | NM_005463 | NP_112740 |
| AV006061 | fatty acid Coenzyme A ligase, | NM_007981 | NP_032007 | DOWN TAC LA | | | |
| | long chain 2 | | | | | | |
| AV140552 | fumarate hydratase 1 | BC006048 | | | DOWN TAC LV | | |
| BG072359 | fumarylacetoacetate hydrolase | NM_010176 | NP_034306 | | DOWN TAC LV | BX537608 | NP_000128 |
| AI841654 | G protein-coupled receptor 56 | NM_018882 | NP_061370 | | DOWN TAC LV | NM_201524 | NP_958933 |
| AV108357 | galactokinase | NM_016905 | NP_058601 | DOWN TAC LA | | BM471434 | NP_000145 |
| AA162908 | gamma-glutamyl transpeptidase | NM_008116 | NP_032142 | DOWN TAC LA | | BC035341 | NP_038347 |
| BG068200 | GATA binding protein 6 | AF179425 | | | DOWN TAC LV | X95701 | NP_005248 |
| BG066689 | glutamate oxaloacetate transaminase | NM_010324 | NP_034454 | DOWN TAC LA | | BM994502 | NP_002070 |
| | 1, soluble | | | | | | |
| AV009064 | glutamine synthetase | NM_008131 | NP_032157 | DOWN TAC LA | | AL161952 | NP_002056 |
| AV134367 | glutaryl-Coenzyme A dehydrogenase | NM_008097 | NP_032123 | | DOWN TAC LV | BC002579 | NP_039663 |
| AV087315 | guanosine monophosphate reductase | NM_025508 | NP_079784 | | DOWN TAC LV | BM994423 | NP_006868 |
| AV022721 | histidine ammonia lyase | NM_010401 | NP_034531 | DOWN TAC LA | | NM_002108 | NP_002099 |
| BG073539 | hydroxysteroid (17-beta) | NM_016763 | NP_058043 | DOWN TAC LA | | BQ940058 | NP_004484 |
| | dehydrogenase 10 | | | | | | |
| BG068774 | isocitrate dehydrogenase 3 | NM_029573 | NP_083849 | DOWN TAC LA | DOWN TAC LV | AK123316 | NP_005521 |
| | (NAD+) alpha | | | | | | |
| AA036340 | isocitrate dehydrogenase 3 | NM_130884 | NP_570954 | DOWN TAC LA | | BQ051868 | NP_777281 |
| | (NAD+) beta | | | | | | |
| AV005828 | L-3-hydroxyacyl-Coenzyme A | NM_008212 | NP_032238 | | DOWN TAC LV | AK096018 | NP_005318 |
| | dehydrogenase, short chain | | | | | | |
| AV022047 | lipin 1 | NM_015763 | NP_766538 | DOWN TAC LA | | AK127039 | NP_663731 |
| AV006290 | lipoprotein lipase | NM_008509 | NP_032535 | DOWN TAC LA | | NM_000237 | NP_000228 |
| BG064854 | low density lipoprotein | AK084165 | | DOWN TAC LA | | NM_004525 | NP_004516 |
| | receptor-related protein 2 | | | | | | |
| AV088662 | malic enzyme, supernatant | NM_008615 | NP_032641 | | DOWN TAC LV | | |
| AV057294 | methylcrotonoyl-Coenzyme A | NM_023644 | NP_076133 | | DOWN TAC LV | BC042453 | NP_064551 |
| | carboxylase 1 (alpha) | | | | | | |
| AA108913 | methylmalonyl-Coenzyme A mutase | NM_008650 | NP_032676 | | DOWN TAC LV | BX647789 | NP_000246 |
| AV006153 | Mus musculus, clone MGC: 7898 | BF180657 | | | DOWN TAC LV | | |
| | IMAGE: 3582717, mRNA, com [symbol] | | | | | | |
| AI854120 | Mus musculus, Similar to | NM_145567 | NP_663542 | DOWN TAC LA | | | |
| | 3-hydroxyisobutyrate dehydrogenase, [symbol] | | | | | | |
| AV088774 | Mus musculus, Similar to | NM_145615 | NP_663590 | DOWN TAC LA | | BM907902 | NP_000117 |
| | electron-transfer-flavoprotein, | | | | | | |
| | alpha p [symbol] | | | | | | |
| AV103083 | NAD(P)H menadione oxidoreductase | NM_020282 | NP_064678 | | DOWN TAC LV | | |
| | 2, dioxin inducible | | | | | | |
| AA162428 | NADH dehydrogenase (ubiquinone) 1 | NM_010885 | NP_035015 | DOWN TAC LA | | | |
| | alpha subcomplex 2 | | | | | | |
| AV016078 | NADH dehydrogenase (ubiquinone) 1 | NM_010885 | NP_035015 | DOWN TAC LA | | | |
| | alpha subcomplex 2 | | | | | | |
| AV140287 | NADH dehydrogenase (ubiquinone) 1 | NM_019443 | NP_062316 | DOWN TAC LA | | | |
| | alpha subcomplex, 1 | | | | | | |
| AV050140 | NADH dehydrogenase (ubiquinone) 1 | BQ044115 | | DOWN TAC LA | | BX538277 | NP_002480 |
| | alpha subcomplex, 4 | | | | | | |
| AV106199 | NADH dehydrogenase (ubiquinone) 1 | NM_025987 | NP_080263 | DOWN TAC LA | DOWN TAC LV | BM709562 | NP_002481 |
| | alpha subcomplex, 6 (14 [symbol] | | | | | | |
| AW555047 | NADH dehydrogenase (ubiquinone) 1 | NM_023202 | NP_075691 | DOWN TAC LA | DOWN TAC LV | BM545518 | NP_004992 |
| | alpha subcomplex, 7 (14 [symbol] | | | | | | |
| AI836747 | NADH dehydrogenase (ubiquinone) 1 | NM_023172 | NP_075661 | DOWN TAC LA | | BM994434 | NP_004996 |
| | beta subcomplex, 9 | | | | | | |
| BG076060 | NADH dehydrogenase (ubiquinone) | BU756147 | | DOWN TAC LA | DOWN TAC LV | | |
| | Fe—S protein 3 | | | | | | |
| AV084172 | ornithine aminotransferase | NM_016978 | NP_058674 | | DOWN TAC LV | BC016928 | NP_000265 |
| BG073162 | oxysterol binding protein-like 1A | NM_020573 | NP_065598 | DOWN TAC LA | | BX647893 | NP_579802 |
| BG071157 | phosphate cytidylyltransferase 1, | AK083965 | | DOWN TAC LA | | BC046355 | NP_005008 |
| | choline, alpha isoform | | | | | | |
| AV033702 | phospholipase A2 group VII | NM_013737 | NP_038765 | DOWN TAC LA | | BC025674 | NP_005075 |
| | (platelet-activating factor acetylhyd [symbol] | | | | | | |
| BG068736 | pyruvate dehydrogenase E1 alpha 1 | NM_008810 | NP_032836 | DOWN TAC LA | | AK092210 | NP_000275 |
| AV012729 | retinoic acid induced 1 | NM_011480 | NP_035610 | DOWN TAC LA | | NM_030665 | NP_109590 |
| AA403731 | RIKEN cDNA 0610009I16 gene | NM_026695 | NP_080971 | DOWN TAC LA | | AL833205 | NP_001976 |
| AI841340 | RIKEN cDNA 0610010E03 gene | NM_025321 | NP_079597 | DOWN TAC LA | | BQ899032 | NP_002992 |
| BG072552 | RIKEN cDNA 0610011L04 gene | NM_177470 | NP_803421 | DOWN TAC LA | | | |
| AV093484 | RIKEN cDNA 0610033L03 gene | NM_026703 | NP_080979 | DOWN TAC LA | DOWN TAC LV | BM704035 | NP_055037 |
| AW558029 | RIKEN cDNA 0710008D09 gene | NM_025650 | NP_079926 | DOWN TAC LA | | | |
| AV086467 | RIKEN cDNA 1010001M12 gene | NM_025348 | NP_079624 | DOWN TAC LA | | BM805609 | NP_004533 |
| AV133828 | RIKEN cDNA 1010001N11 gene | NM_025358 | NP_079634 | DOWN TAC LA | DOWN TAC LV | BM546373 | NP_004993 |
| AV012912 | RIKEN cDNA 1110038I05 gene | NM_134042 | NP_598803 | | DOWN TAC LV | NM_005589 | NP_005580 |
| AV022384 | RIKEN cDNA 1190017B19 gene | NM_023175 | NP_075664 | DOWN TAC LA | | | |
| AV114239 | RIKEN cDNA 1200006L06 gene | NM_024181 | NP_077143 | | DOWN TAC LV | | |
| AV095102 | RIKEN cDNA 1500004O06 gene | NM_025899 | NP_080175 | DOWN TAC LA | | AK094006 | NP_003357 |
| AV052491 | RIKEN cDNA 1810022C23 gene | NM_026947 | NP_081223 | | DOWN TAC LV | | |
| AV063132 | RIKEN cDNA 2210415M14 gene | NM_026219 | NP_080495 | DOWN TAC LA | | BC041005 | NP_006285 |
| AV081301 | RIKEN cDNA 2210418G03 gene | AK008974 | | DOWN TAC LA | | | |
| AV085923 | RIKEN cDNA 2310016C19 gene | NM_025862 | NP_080138 | | DOWN TAC LV | AK125373 | NP_055199 |
| AV086427 | RIKEN cDNA 2310021J10 gene | NM_025641 | NP_079917 | DOWN TAC LA | | | |
| AV103530 | RIKEN cDNA 2310039H15 gene | NM_028177 | NP_082453 | DOWN TAC LA | DOWN TAC LV | BE547177 | NP_004994 |
| AV095143 | RIKEN cDNA 2410004H02 gene | NM_145954 | NP_666066 | DOWN TAC LA | | | |
| BG063257 | RIKEN cDNA 2510027N19 gene | NM_026330 | NP_080606 | DOWN TAC LA | | | |
| AV077867 | RIKEN cDNA 2610003B19 gene | NM_028177 | NP_082453 | DOWN TAC LA | | BE547177 | NP_004994 |
| BG067911 | RIKEN cDNA 2610020H15 gene | NM_025638 | NP_079914 | DOWN TAC LA | DOWN TAC LV | | |
| AV104092 | RIKEN cDNA 2610034N03 gene | NM_025478 | NP_079754 | DOWN TAC LA | | | |
| BG063943 | RIKEN cDNA 2610041P16 gene | NM_025641 | NP_079917 | DOWN TAC LA | | | |
| BG072165 | RIKEN cDNA 2610205J15 gene | NM_152813 | NP_690026 | | DOWN TAC LV | | |
| AV030438 | RIKEN cDNA 2610207I16 gene | NM_024255 | NP_077217 | | DOWN TAC LV | | |
| AV089737 | RIKEN cDNA 3230402N08 gene | NM_021509 | NP_067484 | DOWN TAC LA | | AY007239 | NP_056344 |
| AA154831 | solute carrier family 27 | NM_011978 | NP_036108 | DOWN TAC LA | | D88308 | NP_003636 |
| | (fatty acid transporter), member 2 | | | | | | |
| AA673962 | sortilin-related receptor, LDLR | AF031816 | | DOWN TAC LA | | NM_003105 | NP_003096 |
| | class A repeats-containing | | | | | | |
| AA146030 | sterol carrier protein 2, liver | BC018384 | | DOWN TAC LA | DOWN TAC LV | BX537619 | NP_002970 |
| AV088223 | succinate-CoA ligase, GDP-forming, | NM_019879 | NP_063932 | | DOWN TAC LV | AK125502 | NP_003840 |
| | alpha subunit | | | | | | |
| AV016790 | thioredoxin-like 2 | NM_023140 | NP_075629 | DOWN TAC LA | | AJ010841 | NP_006532 |

What is claimed is:

1. A method for the diagnosis of pressure overload in the heart, the method comprising:
determining the differential expression in one or more of the sequences set forth in Table I.

2. The method according to claim 1, wherein said pressure overload is associated with atrial enlargement and/or ventricular hypertrophy.

3. The method according to claim 1, wherein said determining comprises:
contacting a biological sample comprising protein with an antibody that specifically binds to one or more of the proteins having amino acid sequences encoded by said pressure overload associated genes;
detecting the presence of a complex formed between said antibody and said protein;
wherein an alteration in the presence of said complex, compared to a control sample, is indicative of pressure overload in the heart.

4. The method according to claim 3, wherein said biological sample is blood or serum.

5. The method according to claim 4, wherein said biological sample is contacted with a panel of antibodies specific for pressure overload associated polypeptides.

6. The method according to claim 3, wherein said pressure overload associated genes are set forth in Table II.

7. The method according to claim 5, wherein said biological sample is cardiac cells.

8. The method according to claim 7, wherein said contacting is performed in vivo.

9. The method according to claim 8, the steps comprising:
a) administering to a patient an effective amount of an imaging composition comprising: an antibody that specifically binds to a pressure overload associated polypeptide, and increases contrast between an overloaded cardiac tissue and surrounding tissue in a visualization method; and
b) visualizing said imaging composition.

10. The method according to claim 7, wherein said pressure overload associated genes are set forth in Table III.

11. The method according to claim 1, wherein said determining comprises:
contacting a biological sample comprising protein with a labeled substrate for a metabolic reaction catalyzed by said pressure overload associated genes;
detecting the presence of the product of said metabolic reaction;
wherein an increase in the presence of said complex, compared to a control sample, is indicative of pressure overload in the heart.

12. The method according to claim 11, wherein said pressure overload associated gene is set forth in Table IV.

13. The method according to claim 1, wherein said determining step comprises:
contacting a biological sample comprising nucleic acids from a patient suspected of suffering from pressure overload with a probe that specifically binds to one or more of said sequences;
detecting the presence of a complex formed between said probe and said nucleic acid;
wherein an increase in the presence of said complex, compared to a control sample, is indicative of pressure overload of the heart.

14. The method according to claim 13, wherein said biological sample comprises nucleic acids specifically amplified with said sequences.

15. The method according to claim 13, wherein said biological sample is blood.

16. The method according to claim 13, wherein said biological sample is contacted with a panel of pressure overload associated gene sequences.

17. An array comprising two or more pressure overload associated genes as set forth in Table I, gene products, or antibodies specific for said gene products.

18. A method for identifying an agent that modulates activity of a pressure overload associated gene or gene product, the method comprising:
combining a candidate biologically active agent with any one of:
(a) a polypeptide encoded by any one of the sequences set forth in Table I;
(b) a cell comprising a nucleic acid encoding and expressing a polypeptide encoded by any one of the sequences set forth in Table I; or
(c) a non-human transgenic animal model for pressure overload associated gene function comprising one of: (i) a knockout of a gene corresponding to any one of the sequences set forth in Table I; (ii) an exogenous and stably transmitted mammalian gene sequence comprising any one of the sequences set forth in Table I; and
determining the effect of said agent on pressure overload induced molecular and cellular changes.

19. The method according to claim 18, wherein said biologically active agent upregulates activity.

20. The method according to claim 18, wherein said biologically active agent downregulates activity.

21. The method according to claim 20, wherein said biologically active agent binds to said polypeptide.

22. The method according to claim 1, wherein said sequence is set forth in Table IA.

23. The method according to claim 1, wherein said sequence is set forth in Table IB.
